(19) United States
(12) Patent Application Publication
(10) Pub. No.: US 2024/0115881 A1
(43) Pub. Date: Apr. 11, 2024

(54) METHODS AND SYSTEMS FOR DETERMINING THE DISTRIBUTION OF RADIATION DOSE AND RESPONSE

(71) Applicant: Rutgers, The State University of New Jersey, New Brunswick, NJ (US)

(72) Inventors: Roger W. Howell, Hampton, NJ (US); Sumudu Katugampola, Lincoln, NE (US); Jianchao Wang, Newark, NJ (US)

(73) Assignee: Rutgers, The State University of New Jersey, New Brunswick, NJ (US)

(21) Appl. No.: 18/476,134

(22) Filed: Sep. 27, 2023

Related U.S. Application Data
(60) Provisional application No. 63/377,282, filed on Sep. 27, 2022.

(51) Int. Cl. A61N 5/10

(52) U.S. Cl. CPC A61N 5/1031; A61N 2005/1034

(57) ABSTRACT

A system, method and computer program product to determine radiation dose from radionuclides used to treat a patient in need thereof. The present invention allows users to visualize and understand the impact of radionuclide choice, distribution of activity in the cell, distribution of activity among the cells, cell dimensions, cell separation distance, cluster size, and radiobiological response parameters on the capacity to kill populations of cells. All of these parameters can play a substantial role in determining the surviving fraction of cells. Accordingly, this system, method and computer program product can assist in designing treatment plans for therapeutic radiopharmaceuticals suited to individual needs.

### CROSS-REFERENCE TO RELATED APPLICATIONS

This application claims the benefit of U.S. Provisional Application No. 63/377,282, filed Sep. 27, 2022, the contents of which is incorporated herein by reference in its entirety for all purposes.

### STATEMENT REGARDING FEDERALLY SPONSORED RESEARCH OR DEVELOPMENT

This invention was made with government support under grant number 1R01CA245139 awarded by the National Institutes of Health. The United States government has certain rights to this invention.

### BACKGROUND

The use of chemotherapeutic drugs as an adjuvant to external beam radiotherapy, surgery, or other treatment modalities is common practice for the treatment of a wide variety of solid tumors. This approach has demonstrated some success in the management of certain cancers. The rationale for combining chemotherapeutic agents with external beam radiotherapy is to radio-sensitize the irradiated tumor tissue and/or to target subpopulations of malignant cells that have metastasized from the primary lesion demarcated for beam therapy. Although the tradition of chemoradiotherapy has been practiced for decades and shows promise, some attempts have not succeeded in demonstrating either an added therapeutic benefit or a reduction of normal tissue toxicity. In another approach, radiolabeled chemotherapy agents have been used in an attempt to achieve enhanced cytotoxicity both in human cancer cells and apparently normal hamster fibroblasts. Chemotherapy has also been combined with radioimmunotherapy.

One limitation of chemoradiotherapy is the frequent lack of interaction between chemotherapeutics and ionizing radiation. This often leads to escalation of radiation and drug doses, which in turn, results in elevated normal tissue toxicity. Moreover, lack of specificity of chemotherapy drugs for tumor tissue can result in an insignificant difference in toxicity towards malignant and normal tissues thereby providing no added therapeutic benefit compared to surgery and radiation alone. Despite these limitations, chemoradiotherapy often provides considerable therapeutic benefit. However, observed inconsistencies in treatment outcomes may be due to the widely varying chemotherapeutic drug concentrations employed and radiation absorbed doses achieved. In addition, there is evidence demonstrating that optimization of radiation dose and drug concentration, and the time sequence for administering drugs and radiation play important roles in treatment responses both in vitro and in vivo. Also, regardless of the quality of radiation used, the wide variability in drug toxicity in normal cells of different histologies has to be considered in favor of the most sensitive tissue in chemoradiotherapy. Unfavorable outcomes in therapies involving the use of chemotherapy drugs and radiopharmaceuticals have been attributed to insufficient tumor specificity, poor tumor vascularization, and nonuniformities in agent distribution at the macroscopic, cellular, and subcellular levels. Determination of drug and radionuclide incorporation at the single-cell level has been difficult. As such, estimation of intracellular chemotherapy drug concentration and intra-cellular radioactivity (required to determine radiation absorbed dose to the cell) has largely been restricted to the macroscopic level. Accordingly, it has been difficult to establish a relationship between therapeutic agent incorporation and biologic response.

In addition, the limited success in chemo-radiotherapy of primary solid tumors and metastatic disease is likely due to this lognormal phenomenon, in which minute subpopulations of cells take up very little or no therapeutic agent. Repopulation by these subpopulations could mask a possible treatment benefit and result in an even more resistant neoplastic form. Thus, to enhance tumor response, there continues to be a need to address the nonuniform, lognormal distribution of chemotherapy drugs and radiopharmaceuticals.

Prediction of tumor and normal-tissue responses in therapeutic nuclear medicine relies heavily on calculation of the absorbed dose. A general formalism was developed by the Medical Internal Radiation Dose (MIRD) Committee of the Society of Nuclear Medicine to calculate absorbed doses from tissue-incorporated radioactivity. However, absorbed-dose specification is complex due to the wide variety of radiations emitted, heterogeneity in activity distribution and biokinetics, and other confounding factors. Following the administration of a radiopharmaceutical, the radioactivity is taken up by tumors (if any) and the various organs within the body and the radioactivity is then eliminated through both biological clearance and physical decay.

The extent to which nonuniform distributions of radioactivity within a small tissue element impact the absorbed dose distribution, and ultimately the biological effect, is strongly dependent on the number, type, and energy of the radiations emitted by the radionuclide. Many radionuclides used in nuclear medicine decay by electron capture and/or internal conversion (e.g., ⁶⁷Ga, ^{ppm}Tc, ¹¹¹In, ¹²³I, ²⁰Tl) and consequently emit a large number of low-energy Auger and conversion electrons. Many of these electrons deposit their energy over subcellular dimensions and therefore produce nonuniform dose distributions. Similarly, the short range of alpha particles in biological tissues (40-100 μm) also leads to nonuniform dose distributions from ²²³Ra and other alpha particle emitters of potential use in nuclear medicine. Energetic beta emitters such as ⁹⁰Y have a greater degree of cross-irradiation because their mean range in tissue is at least several hundred microns. However, the nonuniform distribution of these radionuclides invariably leads to nonuniform dose distributions as well. While it is essential to consider the dose distributions that arise from nonuniform distributions of radioactivity, it is also necessary to know whether the dose to a given cell arises from radioactive decays within itself (self-dose) or decays in surrounding cells or other parts of the body (cross-dose). Cellular response to self-dose delivered by a radiopharmaceutical can be considerably different than its response to cross-dose from the same radiopharmaceutical. Accordingly, there is a need for tools and methods that can model biological responses to nonuniform activity distributions encountered in nuclear medicine, to assist in designing therapeutic nuclear medicine treatment strategies for patients undergoing nuclear medicine procedures for cancer therapy.

### BRIEF SUMMARY OF THE INVENTION

An embodiment of the present invention relates to a novel method for predicting the optimal amount of radiopharmaceutical and chemotherapy agents to administer to a patient, by determining the level of cell saturation, geometry of a cluster(s) of cells, and cross dose to a neighboring cell. This includes a method of predicting the response of an individual patient's disease to therapeutic intervention with radiopharmaceuticals, chemo-therapeutics, targeted therapeutics such as radiolabeled monoclonal antibodies, or other agents. Cellular incorporation of therapeutic agents may be measured in the target cell population on a cell-by-cell basis using a flow cytometer. The resulting fluorescence spectra are fitted to the lognormal probability density function to obtain the lognormal shape parameter, σ, also known as the standard deviation, for each treated sample. Surprisingly it has been discovered that: (1) changes in the lognormal shape parameter, σ, upon exposure of the cells to increasing drug concentrations, correlate with changes in the shape of the cell survival curve, and therefore can identify the optimal drug concentration for use in a drug cocktail; (2) the surviving fraction of a target cell population exposed to the therapeutic agent can be predicted using a flow-cytometry assisted Monte Carlo simulation that accounts for the lognormal characteristics of the distribution; and (3) the optimal cocktail of therapeutic drugs can be identified by exposing target cells to combinations of drugs, whereby the optimal concentration of each drug is identified using (1), by employing flow cytometry to simultaneously measure the uptake of each drug, then simulating the surviving fraction of the target population using (2), and using the simulated results to identify the combination of drugs that affords the optimum degree of killing of the target cells. (1) and (2) have been demonstrated with a radiochemical (²¹⁰Po-citrate) and two anticancer drugs (daunomycin and doxorubicin) in Chinese hamster V79 cells. (3) has been demonstrated with a combination of ²¹⁰Po and daunomycin and a combination of ²¹⁰Po and doxorubicin. Another aspect of the invention provides patient-specific cocktail formulations by identifying existing drugs that can be added to a cocktail to facilitate targeting subpopulations of cells that would otherwise escape targeting.

An additional embodiment of the invention is directed to a computing-implemented method of determining a dose of radiation for a patient for radiation therapy treatment planning, the method comprising: receiving, by a computing device, one or more features associated with a dose of radiation; displaying, by the computing device, a menu of one or more of the features associated with the dose of radiation, wherein each feature corresponds to a category of information; receiving, by the computing device from the user, a selection of one or more feature from the menu of features; and for each selected feature: receiving, by the computing device, information pertaining to the selected feature, determining, by the computing device radiation data for a radionuclide, determining, by the computing device a radiobiological parameter, determining, by the computing device a target volume and source volume, determining, by the computing device a cell geometry, and determining, by the computing device, a dose of radiation based upon the selected features. In a further embodiment, displaying a menu of one or more features comprises displaying a menu comprising an option associated with one or more of the following: a source of radiation; a cell source; a radiobiological parameter; and a cell geometry feature.

In another embodiment, the source of radiation feature is a radionuclide or a monoenergetic particle. In a further embodiment, the cell source feature comprises a target volume in a cell for which radiation absorbed dose will be calculated. In another embodiment, the radiobiological parameter feature comprises select values for calculating the probability that a given cell survives using the linear quadratic model. In a further embodiment, the cell geometry feature comprises one of the following: a one dimensional cell pair, a cell population that resides on a plane, or a three dimensional configuration of cells. In another embodiment, the radiation data for a radionuclide feature further comprises select values for radiopharmaceutical agents. In a further embodiment, the radiobiological parameter feature comprises select values for calculating the probability that a given cell survives using the linear quadratic model, and then determining the surviving fraction of cells within a colony of cells or a three dimensional cluster of cells. In another embodiment, the cell geometry feature comprises the distribution of activity among the cells that is based on data from a patient or from a laboratory.

An additional embodiment of the invention is directed to a radiation therapy planning system configured to determine a dose of radiation for a patient for radiation therapy treatment planning, the system comprising: a computing device; and a computer-readable storage medium in communication with the computing device, wherein the computer-readable storage medium comprises one or more programming instructions that, when executed, causes the computing device to: receive, one or more features associated with a dose of radiation from a computing source; display, a menu of one or more of the features associated with the dose of radiation, wherein each feature corresponds to a category of information; receive, a selection of one or more feature from the menu of features; and for each selected feature: receive information pertaining to the selected feature, determine radiation data for a radionuclide; determine a radiobiological parameter; determine a target volume and source volume; determine a cell geometry; and determine a dose of radiation based upon the selected features.

In a further embodiment, one or more programming instructions that, when executed, cause the computing device to display a menu of one or more features comprise one or more programming instructions that, when executed, cause the computing device to display a menu comprising an option associated with one or more of the following: a source of radiation; a cell source; a radiobiological parameter; and a cell geometry feature. In another embodiment, the source of radiation feature is a radionuclide or a monoenergetic particle. In a further embodiment, the cell source feature comprises a target volume in a cell for which radiation absorbed dose will be calculated. In another embodiment, the radiobiological parameter feature comprises select values for calculating the probability that a given cell survives using the linear quadratic model. In a further embodiment, the cell geometry feature comprises one of the following: a one dimensional cell pair, a cell population that resides on a plane, or a three dimensional configuration of cells. In another embodiment, the radiation data for a radionuclide feature further comprises select values for radiopharmaceutical agents. In a further embodiment, the radiobiological parameter feature comprises select values for calculating the probability that a given cell survives using the linear quadratic model, and then determining the surviving fraction of cells within a colony of cells or a three dimensional cluster of cells. In another embodiment, the distribution of activity among the cells is based on data from a patient or from a laboratory.

In some examples, receiving a selection of one or more features from the menu of features includes receiving a selection of source of a radiation feature; wherein the source of radiation feature is a predefined MIRD radionuclide, a monoenergetic particle, or a user-created radionuclide.

In some examples, receiving a selection of one or more features from the menu of features includes receiving a selection of a cell source feature; wherein the cell source feature comprises a target volume in a cell for which radiation absorbed dose will be calculated, wherein the source region comprises a cell, a cell nucleus, cytoplasm, or a cell surface.

In some examples, receiving a selection of one or more features from the menu of features includes receiving a radiobiological parameter feature; wherein the radiobiological parameter feature comprises select values for calculating the probability that a given cell survives using a linear quadratic model, wherein the linear quadratic model comprises separate parameters for each type of radiation and separate parameters for each target region.

### BRIEF DESCRIPTION OF THE DRAWINGS

FIG. 1 shows the distribution of cellular uptake of citrate, daunomycin, and doxorubicin by V79 cells in a suspension culture. Displayed are representative flow cytometry generated histograms of cellular fluorescence intensity after treatment with 0-3 mmol/L EuTc-citrate (A), 0-10 μmol/L daunomycin (B), or 0-10 μmol/L doxorubicin (C).

FIG. 2 shows the least squares fits of the flow cytometry fluorescence intensity histograms to a lognormal probability distribution. The histograms correspond to (A) EuTc-citrate, (B) daunomycin, and (C) doxorubicin.

FIG. 3A displays net mean fluorescence intensity (MFI) of europium tetracycline-citrate complex (EuTc-citrate) as a function of extracellular citrate concentration (open circle, solid line) and corresponding mean ²¹⁰Po activity per cell (filled circle, dashed line). Lines represent least squares fits of the data to linear functions: MFI=267+16(mmol/L)⁻¹×C_{cit}, and MFI=1058+19(mBq/cell)⁻¹×C<a₀>, where C_{cit} and <a₀>are the extracellular citrate concentration and mean cellular activity of ²¹⁰Po, respectively. FIG. 3B displays net MFI of intracellular daunomycin after exposure to low extracellular concentrations (filled square, dashed line) and high concentrations (open square, solid line). Linear least squares fits to the data give MFI_{dauno}(C_{dauno}<0.6 μmol/L)=5922 (μmol/L)⁻¹×C_{dauno} and MFI_{dauno} (C_{dauno}>0.6 μmol/L)=2480(μmol/L)⁻¹×C_{dauno}+1161. FIG. 3C displays net MFI of intracellular doxorubicin after exposure to low extracellular concentrations (filled triangle, dashed line) and high concentrations (open triangle, solid line). Linear least squares fits to the data give MFI_{doxo}(C_{doxo}<1 μmol/L) =61 (μmol/L)⁻¹×C_{doxo} and MFI_{doxo}(C_{doxo}>1 μmol/L)=40(μmol/L)⁻¹×C_{doxo}+29. For all cases, error bars represent standard error (SE) of three independent experiments.

FIGS. 4-C show the surviving fraction (SF) of V79 cells after treatment with various agents. FIG. 4A displays ²¹⁰Po-citrate for three independent experiments, SF plotted against absorbed dose to the cell nucleus, intracellular ²¹⁰Po activity, and net mean fluorescence intensity (MFI) of the europium tetracycline-citrate complex. Data plotted are from three independent experiments. Curve represents a least squares fit of the data to a single component exponential function. FIG. 4B displays daunomycin (open square), SF plotted against extracellular drug concentration and against net MFI of the drug. FIG. 4C displays doxorubicin (open triangle), SF plotted against extra-cellular drug concentration and against net MFI of the drug. Curves for daunomycin and doxorubicin represent least-squares fits to a two-component exponential function. For ²¹⁰Po-citrate, horizontal and vertical error bars represent SE of mean cellular activity and surviving fraction of triplicate measurements, respectively. For daunomycin and doxorubicin, horizontal and vertical error bars represent SE of Net MFI and surviving fraction for three independent experiments.

FIG. 5 shows a flow chart of the Monte Carlo procedure for determining fraction of surviving cells based on cellular fluorescence intensity profiles of the incorporated agent. In STEP 1, flow cytometry was used to obtain fluorescence intensity of EuTc-citrate (²¹⁰Po-citrate), daunomycin, and doxorubicin in individual V79 cells in a suspension culture. The distribution of measured cellular fluorescence intensities, adapted from FIG. 1, is shown in (A) 0.1-3 mM citrate, (B) 0-10 μM daunomycin, or (C) 0-10 μM doxorubicin. After calculating the probability of survival Pᵢ(I′ᵢ) for the i^{th} cell based on the normalized fluorescence intensity I′ᵢ (STEP 2), a random number RANDᵢ(0<RANDᵢ≤1) was generated as depicted in STEP 3 by the dice. If RANDᵢ<Pᵢ(I′ᵢ), then the cell was scored as a survivor, otherwise it was considered dead (STEP 4). A surviving cell is represented by a blossoming tree with leaves, while a dead cell is depicted by a tree without leaves. By repeating STEPS 1-4 for every cell in each population, the surviving fraction for any sample population was calculated as illustrated in STEP 5.

FIGS. 6A-C show comparison of Monte Carlo simulated cell survival (light symbols) with experimental clonogenic cell survival (black symbols) of V79 cells after treatment with (A) ²¹⁰Po-citrate, (B) daunomycin, or (C) doxorubicin. The surviving fraction for ²¹⁰Po-citrate are plotted against mean absorbed dose to the cell nucleus, mean intracellular ²¹⁰Po activity, and mean fluorescence intensity of the europium tetracycline-citrate complex. Dashed lines represent Monte Carlo simulations of cell survival when every cell in the population is assumed to contain the same amount of drug that corresponds to the mean drug uptake for the respective extracellular concentration (i.e., net mean fluorescence intensity). Error bars represent the SE for <I>ₙₑₜ based on fluorescence data from two and three independent experiments for daunomycin and doxorubicin, respectively. Error bars for ²¹⁰Po citrate data are smaller than the symbols.

FIG. 7A displays the lognormal shape parameters σ for ²¹⁰Po-citrate,

daunomycin, and doxorubicin plotted against intracellular ²¹⁰Po activity (filled circle, solid line), and extracellular concentration of daunomycin (open square, dashed line) and doxorubicin (open triangle, dotted line), respectively. FIG. 7B displays the surviving fraction versus shape parameter for ²¹⁰Po-citrate (filled circle), daunomycin (open square), and doxorubicin (open triangle). Error bars represent SE of three independent experiments.

FIG. 8 shows a flow chart of the Monte Carlo procedure for determining fraction of surviving cells based on cellular fluorescence intensity distributions that arise after treatment of cells with a cocktail of two therapy agents (Agent 1 and Agent 2). In STEP 1, flow cytometry is used to obtain a fluorescence intensity dot-plot of the treated cells. STEP 2 calculates the probability P(I′ᵢ₁, I′ᵢ₂) that the i^{th} cell survives treatment with Agents 1 and 2, based on the respective normalized fluorescence intensities I′ᵢ₁ and I′ᵢ₂. A random number RANDᵢl (0<RANDᵢ≤1) is then generated as depicted in STEP 3 by the dice. If RANDᵢ<P(′ᵢ₁, I′ᵢ₂), then the cell is scored as a survivor, otherwise it is considered dead (STEP 4). By repeating STEPS 1-4 for every cell, the surviving fraction for any sample population is calculated as illustrated in STEP 5.

FIG. 9 displays the surviving fraction of V79 cells after treatment with graded amounts of ²¹⁰Po-citrate in the absence or presence of: (A) 0.63 μM daunomycin or (B) 2.50 μM doxorubicin. Three independent experiments (open circle: ²¹⁰Po-citrate; filled square: ²¹⁰Po-citrate+daunomycin; filled triangle: ²¹⁰Po-citrate+doxorubicin). Dashed lines represent least-squares fits of data for ²¹⁰Po-citrate to a 1-component exponential function. Solid curves represent least-squares fits of data for the combined treatment to 2-component exponential functions. Correction of the combined treatment curves for drug toxicity yielded the dotted curves. Horizontal and vertical error bars represent SE of mean cellular activity and surviving fraction of triplicate measurements, respectively. Some error bars are smaller than the symbols.

FIGS. 10A-B display the comparison of Monte Carlo simulated cell survival (symbols) with experimental clonogenic cell survival (solid curves) of V79 cells after treatment with combinations of (A) ²¹⁰Po-citrate+0.63 μM daunomycin or (B) ²¹⁰Po-citrate+2.50 μM doxorubicin. The experimental survival curves are least squares fits of the data presented in FIG. 9. Open and closed symbols represent Monte Carlo simulations of cell survival when the agents are assumed to act independently and interactively, respectively. Error bars represent the SE for simulated surviving fraction based on fluorescence data from two independent experiments for each cocktail. Some error bars are smaller than the symbols.

FIG. 11 illustrates a block diagram of an example system for determining a dose of radiation according to an embodiment.

FIG. 12 illustrates a flow chart of an example for a method of determining a dose of radiation according to an embodiment for an application.

FIG. 13 illustrates an example graphical user interface (GUI) of the Source Radiation feature. This feature provides three major options for selecting the type of radioactivity to be placed in the labeled cells: 1) Predefined MIRD Radionuclide (upper left). Radiation spectra are available for predefined radionuclides that include either average beta particle energies (β Average Energy Spectrum) or the complete beta particle spectrum (β Full Energy Spectrum). 2) Monoenergetic Particle Emitter (upper right). Here, the user can select either an alpha particle or electron, and specify the particle yield per disintegration and energy. 3) User-Created Radionuclide (lower left). The user can create a radionuclide that includes a variety of selectable radiations (alpha, Auger electron, beta minus, and beta plus). Once the radionuclide is selected, its corresponding data are streamed into the lower right box entitled Input Data for Calculation, that a user may use to create a new application according to an embodiment.

FIG. 14 illustrates an example GUI of a Cell Source/Target feature. The source region (red/dark grey) in the cell that contains the radiopharmaceutical can be selected as cell, cell surface, nucleus, or cytoplasm (top left). Selectable target regions (blue/light grey) include cell, nucleus, or cytoplasm (top left). The cell and cell nucleus are represented by concentric shells of unit density water with cell radius (R_{C}) and cell nucleus radius (R_{N}), which can be set as desired (bottom left). The selections made by the user are then depicted (right) that a user may use to identify one or more features associated with an application according to an embodiment.

FIG. 15 illustrates an example GUI of a Radiobiological Parameters feature. The self-dose and cross-dose is tallied for each cell in the multicellular geometry. The probability that a given cell survives is calculated using the linear quadratic model. The responses to self-dose and cross-dose can be set independently using the parameters α_{self}, β_{self} and α_{cross}, β_{cross}, respectively.

FIG. 16 illustrates an example GUI of a Multicellular Geometry <1-D Cell Pair sub-feature. This sub-feature enables rapid calculation of the self-dose to a labeled cell and cross-dose to a neighboring cell that lies at some distance (i.e., 16 μm between centers in this example). The self- and cross- doses per unit cumulated activity in the source cell (Gy Bq⁻¹ s⁻¹), also known as S value, are reported for the selected source radiation (i.e., Y-90 in this example) in the box labeled Result. The calculation can be repeated for different cell separation distances (top left).

FIG. 17A illustrates an example GUI of a Multicellular Geometry <2-D Colony <2-D Colony sub-feature. The user can select a multicellular geometry wherein the cell population lies on a plane. In the Cell Geometry box, the cell population can be constrained to different selectable shapes including circle (shown), ellipse, and rectangle (upper left). Dimensions of each shape are provided by the user (i.e., circle with 66 μm radius in this example). In the Cell Labeling box, the activity can be distributed amongst the cell population according to selectable labeling methods: uniform (shown), normal, and lognormal. The mean activity per cell, residence time, and percentage of cells that are labeled can be specified. Upon selecting parameters and selecting Compute, the resulting multicellular geometry is plotted on the right in a manner that indicates whether a cell is labeled (red/dark grey) or unlabeled (green/light grey), and the transparency represents whether the cell is dead (transparent) or alive (opaque), an example analytic chart according to an embodiment.

FIG. 17B illustrates an example GUI of additional geometries available in Multicellular Geometry <2-D Colony <2-D Colony. These are 2-D Colony shapes containing 1000 cells, with 50% of the cells labeled with Y-90. Circular, ellipsoidal, and rectangular colony shapes are shown as examples. Cells are labeled (red/dark grey) or unlabeled (green/light grey), and the transparency represents whether the cell is dead (transparent) or alive (opaque).

FIG. 18 illustrates an example GUI of a Multicellular Geometry <2-D Colony <Surviving Fraction Curve. Selecting the Surviving Fraction Curve sub-feature provides a survival curve for the cell population under consideration based on entries in the tabs. Using the drop-down menu labeled “Select the Curve,” located below the abscissa, three different plots are available: 1) Surviving fraction of mixed population of cells versus mean absorbed dose to all cell nuclei, 2) Surviving fraction of labeled cells versus mean absorbed dose to labeled cells, and 3) Surviving fraction of unlabeled cells versus mean absorbed dose to unlabeled cells.

FIG. 19A illustrates an example GUI of a Multicellular Geometry <2-D Colony <Activity Distribution Histogram. Selecting the Activity Distribution Histogram sub-feature provides the distribution of activity among the labeled cell population under consideration based on entries in the sub-feature. This example shows a uniform distribution of activity among the labeled cells (each labeled cell has same activity).

FIG. 19B illustrates examples of the different cellular activity distributions that can be selected. Initial activity histogram for labeled cells: a) Uniform Distribution, b) Normal Distribution with mean activity per cell equal to 1 Bq and standard deviation equal to 0.1 Bq , c) Lognormal Distribution with mean activity per cell equal to 1 Bq and shape factor equal to 1. These distributions are also available in the 3-D Cluster.

FIG. 20A illustrates an example GUI of a Multicellular Geometry <3-D Cluster<3-D Cluster. Here, the user can select a multicellular geometry wherein the cell population is contained within a three-dimensional geometry. In the Cell Geometry box, the cell population can be constrained to different selectable shapes including sphere (shown), ellipsoid, and cone. Dimensions of each shape are provided by the user (i.e., sphere with 65 μm radius in this example). In the Cell Labeling box, the activity can be distributed amongst the cell population according to selectable labeling distributions: uniform, normal, and lognormal (shown). The mean activity per cell, residence time, and percentage of cells that are labeled can be specified. Upon selecting parameters and selecting “Compute,” the resulting multicellular geometry is plotted on the right in a manner that indicates whether a cell is labeled (red/dark grey) or unlabeled (green/light grey), and the transparency represents whether the cell is dead (transparent) or alive (opaque).

FIG. 20B illustrates examples of additional geometries available in Multicellular Geometry <3-D Cluster<3-D Cluster. These are 3-D Cluster shapes containing 10000 cells, with 50% of the cells labeled with Y-90, a user may define the amount of cells per cluster, several to in excess of 100,000 cells. Cluster shapes are Sphere, Rod, Cone, and Ellipsoid. Cells are labeled (red/dark grey) or unlabeled (green/light grey), and the transparency represents whether the cell is dead (transparent) or alive (opaque).

FIG. 21 illustrates an example GUI of a Multicellular Geometry <3-D Cluster <Surviving Fraction Curve. Selecting the “Surviving Fraction Curve” sub-feature provides a survival curve for the cell population under consideration based on entries in the sub-feature. Using the drop-down menu labeled “Select the Curve,” located below the abscissa, three different plots are available: 1) Surviving fraction of mixed population of cells versus mean absorbed dose to all cell nuclei, 2) Surviving fraction of labeled cells versus mean absorbed dose to labeled cells, and 3) Surviving fraction of unlabeled cells versus mean absorbed dose to unlabeled cells.

FIG. 22 illustrates an example GUI of a of Multicellular Geometry <3-D Cluster <Activity Distribution Histogram. Selecting the Activity Distribution Histogram sub-feature provides the distribution of activity among the labeled cell population under consideration based on entries in the tabs. This example shows a lognormal distribution. The small number of labeled cells (in this case it is 172) magnifies the stochastic aspects of the distribution in such a small population. Repeated selection of the Compute feature shows variations in the distribution when the Monte Carlo calculations are repeated.

FIG. 23 illustrates an example GUI of an Output feature. This is a tabulation of the mean absorbed dose per unit cumulated activity (S value) for the various target and source volumes in cells of the dimensions selected in the Cell Source/Target feature. In this example, the cell radius is 6 μm which is half of the minimum cell separation distance of 12 μm (row 12). The top row provides the S values for a labeled cell. The cross-dose S values are provided by row 12 and higher. The distance represents the distance between the two cells for which the cross-dose S value is being calculated.

FIG. 24 illustrates an example GUI of an Information feature.

FIG. 25 illustrates a block diagram of example hardware that may be used to contain or implement program instructions according to an embodiment.

FIG. 26 illustrates a flow chart of an example method of determining a dose of radiation for a patient according to an embodiment. As illustrated by FIG. 26, the method may include receiving by a computing device 2600 one or more features associated with a dose of radiation. In an embodiment, the computing device may display 2602 a menu of one or more of the features associated with the dose of radiation. One or more of the features may correspond to a category of information.

FIG. 27 illustrates an example GUI for an enhanced Source Radiation feature.

FIG. 28 illustrates an example GUI for an enhanced Cell Source/Target feature.

FIG. 29 illustrates an enhanced GUI for a Radiobiological Parameters feature.

FIG. 30 illustrates an enhanced GUI for a Multicellular Geometry feature.

FIG. 31 illustrates an enhanced 3-D Cluster sub-feature.

FIG. 32 illustrates Tomographic section through the center of the spherical cell cluster.

FIG. 33 illustrates a comparison between predictions and experimental observations.

FIG. 34 illustrates Multicellular Geometry parameters.

FIG. 35 illustrates a comparison between predictions and experimental observations.

FIG. 36 illustrates a comparison between predictions and experimental observations.

FIG. 37 illustrates activity distributions.

FIG. 38 illustrates surviving fraction curves.

FIG. 39 illustrates a cell nucleus model.

FIG. 40 illustrates a comparison between simulations.

FIGS. 41-48 illustrate surviving fraction curves.

FIGS. 49-52 illustrate comparisons between simulations.

### DETAILED DESCRIPTION

The therapeutic significance of nonuniform incorporation of chemotherapy drugs and radiopharmaceuticals by cancer cells has been recognized as an issue of long standing in the art. Yet, the impact of lognormal drug distributions on the capacity of an agent to sterilize a population of cells has not been previously recognized. A small lognormal shape parameter (σ) implies a narrow distribution profile, and σ approaches zero when all cells incorporate the same amount of agent. On the other hand, a large σ signifies a wide spread in distribution, and agent incorporation may range from very low (potentially nontoxic) to high (lethal). The ubiquity of lognormal drug distributions has now been demonstrated by using flow cytometry to assess the distribution of radionuclides, for example, ²¹⁰Po-citrate, and pharmaceutical agents, for example, daunomycin, and doxorubicin. Equally important is the discovery that changes in the value of σ as a function of increasing drug concentration parallel marked changes in the shapes of the corresponding clonogenic cell survival curves. Further, surprisingly it has now been discovered that experimental lognormal distributions (i.e., individual drug uptake on a cell-by-cell basis) can be used to accurately predict the saturation that is observed in experimental cell survival-curves (e.g., two-component exponential curves). This saturation has now been observed repeatedly in studies on the lethal effects of nonuniform distributions of radioactivity. Furthermore, theoretical studies now show that lognormal distributions can lead to such two-component exponential survival curves in both monolayer and three-dimensional tissue constructs.

The overall biological response must be influenced by the magnitude of the mean cellular drug uptake and the degree of heterogeneity in agent distribution. Therefore, a change in the capacity of an agent to sterilize a cell population is related to both the change in width of the distribution and the peak-shift as the agent concentration increases. While the former is a measure of the broadness of a distribution profile of the agent among a cell population and can be represented by the lognormal shape parameter, σ, the latter is a shift in the lognormal scale parameter μ which is an indication of cells accumulating increasing levels of the agent. Changes in σ are prognostic of whether a survival curve will exhibit saturation, and that σ may guide in the selection of agents for multimodality cocktail design by providing information on agent concentrations at which the first component of cell kill ends. However, the shape parameter only describes the agent distribution profile of the cell population as a whole, but does not provide information on the fate of individual cells of the population.

Surprisingly, it has also now been discovered that clonogenic cell survival can be predicted based only on knowledge of the initial slope of the cell survival curve and information on the distribution of agent incorporation among the treated cell population. For example, the distribution of ²¹⁰Po, daunomycin and doxorubicin among populations of Chinese hamster V79 cells was assessed using flow cytometry techniques and used to theoretically model the surviving fraction. Several modeling approaches were compared, including flow-cytometry gating of agent-negative cells, Monte Carlo simulation of cell survival based on the experimental distributions of drug uptake, and Monte Carlo simulation of cell survival based on the more conventional approach of using mean cellular uptake of the drugs.

Monte Carlo simulation using cellular agent incorporation based on individual cell fluorescence intensity of therapeutic agents is a suitable predictor of cell survival. This flow cytometry based approach, which takes explicit account of the lognormal distribution of cellular uptake of the agents, offers a rapid means for determining treatment response on a cell-by-cell basis, and allows the selection of agents for the design of highly effective therapeutic cocktails that are capable of targeting the diversity in tumor cell populations. Such cocktails can be created not only for treatment of cancer, but also for infectious diseases and other diseases that may be amenable to targeted therapies. Furthermore, this single-cell Monte Carlo technique can be used to resolve difficulties encountered when attempting to predict biological response at the multicellular level using macroscopic mean agent doses.

An embodiment of the invention is directed to a method for predicting the response of an individual patient's cells to therapeutic intervention comprising the steps of:
(a) exposing populations of said cells to increasing concentrations of a candidate therapeutic agent for said condition;
(b) measuring the incorporation of said therapeutic agent in said populations on a cell-by-cell basis, preferably employing a high-speed technique, preferably flow cytometry, with the incorporation being measured using fluorescence spectroscopy, preferably the individual cell fluorescence intensities and the mean fluorescence intensity (MFI);
(c) plotting the number of cells versus the amount of incorporated therapeutic agent, to obtain distribution plots for said populations;
(d) fitting said distribution plots to a probability density function, preferably a lognormal function, to obtain a distribution curve and the standard deviation, σ, for each population; and
(e) identifying the optimal concentration of said therapeutic agent for said patient by identifying changes in the slope of a plot of σ as a function of concentration of said therapeutic agent.

The method may further comprise the steps of determining the surviving fraction of cells, plotting the surviving fraction versus the amount of incorporated therapeutic agent, and fitting the plot to a probability function, preferably selected from the group of functions consisting of exponential and linear-quadratic. Preferably, the method also further comprises the step of predicting the surviving fraction of said cell populations using a simulation, preferably a Monte Carlo simulation, that accounts for the characteristics of said distribution curve, preferably flow-cytometry assisted Monte Carlo simulation.

In a further embodiment of the invention, cells are exposed to increasing concentrations of a plurality of therapeutic agents, and the optimal concentration of each drug is identified. The simulation results are then used to identify an effective combination of therapeutic agents in their therapeutically effective amounts.

The biological targets of the method include cells with uncontrolled growth, such as tumor cells, or cells infected with pathogens, including without limitation, bacteria, viruses, prions, and parasites. The biological target may also include stem cells. In a preferred embodiment of the method, the individual patient's cells comprise cancer cells.

The therapeutic agents comprise, without limitation, antibodies, peptides, chemo-therapeutics, radiopharmaceuticals, antifungals, antibiotics and other pharmaceuticals.

Any high-speed technique for assaying drug uptake on a cell-by-cell basis, as known in the art, can be used, including, without limitation, microfluidic techniques such as flow cytometry and microfluidic impedance cytometry; laser scanning microscopy; and gas chromatography/mass spectrometry (GC/MS). Preferably, the high-speed technique for assaying therapeutic agent uptake on a cell-by-cell basis comprises flow cytometry. The analytical method used to determine the incorporation of therapeutic agent preferably comprises fluorescence spectroscopy. The fluorescence measurement preferably comprises individual cell fluorescence intensities and the mean fluorescence intensity (MFI).

Preferably, the probability density function of step (d) is selected from the group of functions consisting of lognormal, normal, Weibull and exponential. The probability function chosen will have some impact on the value of σ, and therefore will have some impact when determining the optimal concentration from plots of σ versus concentration. Most preferably, the probability density function is lognormal. However, the simulation of the surviving fraction can also directly employ the incorporation data (e.g., flow cytometry data) without relying on a lognormal or other function fit to obtain σ.

The probability function of the plot of surviving cell fraction versus the amount of incorporated therapeutic agent can be any typical dose-response function. Preferably this survival probability function is selected from the group of functions consisting of exponential and linear-quadratic, and is most preferably exponential.

Preferably, the Monte Carlo simulation method comprises flow-cytometry assisted Monte Carlo simulation.

In a further embodiment of the invention is directed to a method for predicting the response of an individual patient's cancer cells to therapeutic intervention comprising the steps of:
(a) exposing populations of said cancer cells to increasing concentrations of a candidate therapeutic agent for said cancer;
(b) measuring by fluorescence spectroscopy the incorporation of said therapeutic agent in said populations on a cell-by-cell basis using a flow cytometer, to provide net mean fluorescence intensity (MFI);
(c) plotting the number of cells versus the net MFI, to obtain a distribution plot for said population;
(d) fitting said distribution plot to a lognormal probability density function to obtain a lognormal distribution curve and the standard deviation, σ, for each population; and
(e) identifying the optimal concentration of said therapeutic agent for said cancer patient from changes in slope of a plot of σ as a function of concentration of said therapeutic agent.

Preferably, the method further comprises the step of predicting the surviving fractions of said cell populations using a flow-cytometry assisted Monte Carlo simulation that accounts for the characteristics of said lognormal distribution curve.

In a further embodiment of the invention, the cancer cells are exposed to increasing concentrations of a plurality of therapeutic agents, and the optimal concentration of each drug/agent is identified, and the simulation results are used to identify a combination of therapeutic agents that affords a high degree of killing of the cancer cells. Preferably the degree of killing of the cancer cells is about 99% or greater, more preferably 99.9% or greater, and most preferably 99.99% or greater. The method can also be used to identify a combination of drugs that affords the optimum degree of killing of the cancer cells.

In yet another embodiment of the invention, the method further comprises the step of identifying one or more drugs that can be added to a combination of therapeutic agents to facilitate the killing of subpopulations of cells that would otherwise escape killing by said combination.

Yet another embodiment of the invention comprises a method of high-throughput drug discovery comprising the method described above for predicting the response of an individual patient's cells to therapeutic intervention. Such an embodiment can be implemented on a high-throughput drug discovery platform. For example, in one embodiment, a tissue sample from a patient would be cultured and loaded into a high-throughput drug discovery device which is coupled to a flow cytometer, numerous combinations from a library of drugs would be screened, and a cocktail specific for the patient at hand would be identified.

Still another embodiment of the invention is directed to a 2-stage targeting method of treating a disease or condition for a patient in need thereof, the method comprising:
(1) identifying and providing a plurality of candidate targeting agents relevant to the disease or condition of said patient, wherein said targeting agents are two-stage agents comprising:
   (a) Stage 1 agents which are non-toxic and target the diseased or affected cells; and
   (b) Stage 2 agents which bind to said Stage 1 agents and carry at least one additional agent selected from the group consisting of toxins, radionuclides and fluorochromes, wherein each Stage 2 agent can only bind to a single corresponding Stage 1 agent;
(2) injecting said patient with a cocktail of Stage 1 agents via a route appropriate to said disease or condition, and allowing sufficient time for maximum uptake by said diseased or affected cells and substantial clearance of unbound Stage 1 agents;
(3) withdrawing a sample of said patient's diseased or affected cells loaded with Stage 1 agents;
(4) treating said sample of said cells in vitro with a cocktail of said Stage 2 agents, wherein each Stage 2 agent carries a unique fluorochrome, and wherein said Stage 2 agents bind to said Stage 1 agents loaded into said cells;
(5) quantifying the amount of each Stage 2 agent binding to each diseased or affected cell using fluorescence spectroscopy;
(6) predicting the response of said diseased or affected cells for every possible combination of Stage 1 and Stage 2 agents using the above method for predicting the response of an individual patient's cells to account for the lognormal distribution of each agent, and identifying the optimal combination of said agents;
(7) arming each Stage 2 agent of said optimal combination with one or more therapeutic agents selected from the group consisting of toxins, radionuclides, and combinations or two or more thereof, to form an armed cocktail;
(8) optionally, repeating step (2); and
(9) injecting said armed cocktail into said patient.

The method may further comprise repeating steps (3) through (6) with healthy cells of said patient in place of diseased/affected cells, in order to assess the uptake of said Stage 1 and Stage 2 agents in each healthy cell.

A further embodiment of the invention is directed to a computational method for processing the above-indicated data, including flow cytometry data, in order to determine the parameter σ and calculate therefrom the optimal dose, or effective dose, of each component of the drug cocktail.

In addition, the above-identified methods can be used in radioimmunotherapy/chemotherapy to predict the toxicity of cocktails of α-emitting radiopharmaceuticals and chemotherapy drugs in a manner that takes into account the effects of lognormal and other nonuniform distributions of agents within cell populations. These agents can interact with one another and cause greater than expected effects based on their single-agent toxicities. The approach is employed advantageously in the selection of agents for the design of highly effective α-particle based therapeutic cocktails that are capable of targeting the diversity in tumor cell populations.

The above-identified methods have the capacity to predict clonogenic survival after multi-modality therapy, using flow cytometry-assisted Monte Carlo simulation. It is demonstrated herein that Monte Carlo simulation using cellular agent incorporation based on individual cell fluorescence intensities of therapeutic agents is a suitable predictor of cell survival. This model accounts for the lognormal distribution of cellular uptake of the agents, and is capable of predicting treatment response on a cell-by-cell basis.

### Cellular Uptake of ²¹⁰Po-Citrate, Daunomycin, and Doxorubicin

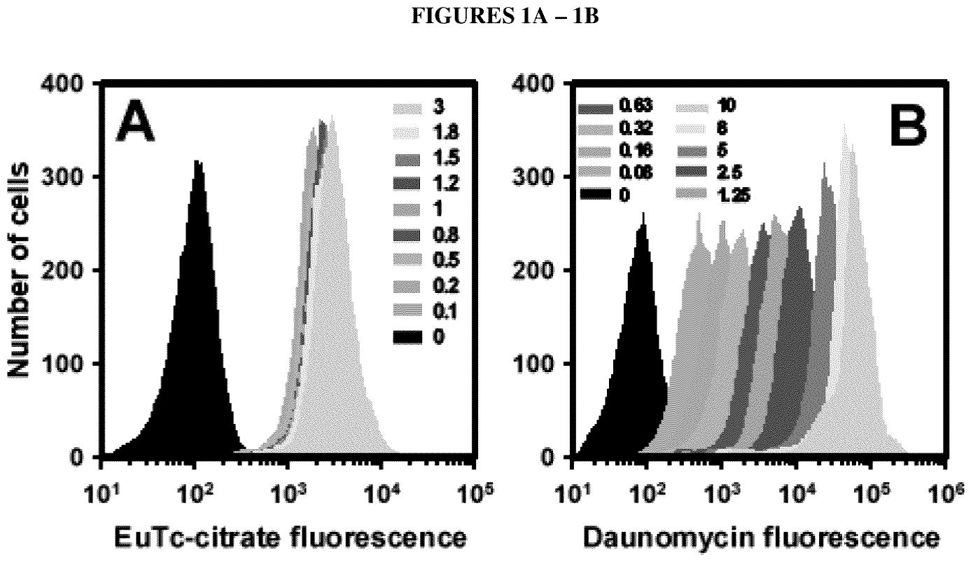

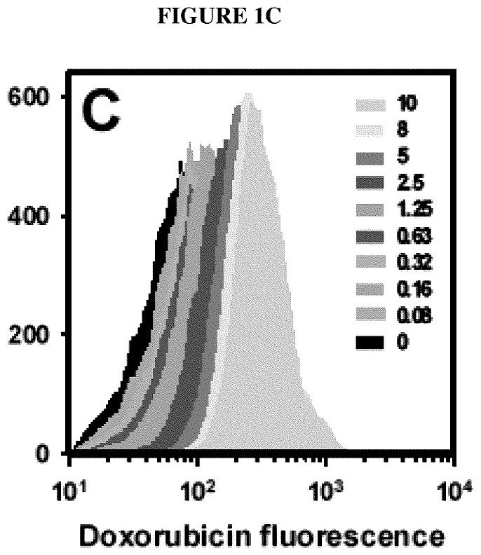

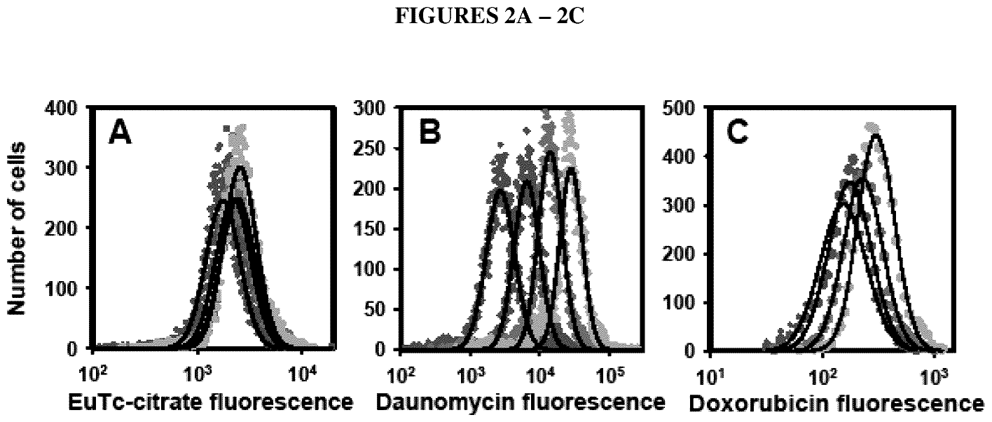

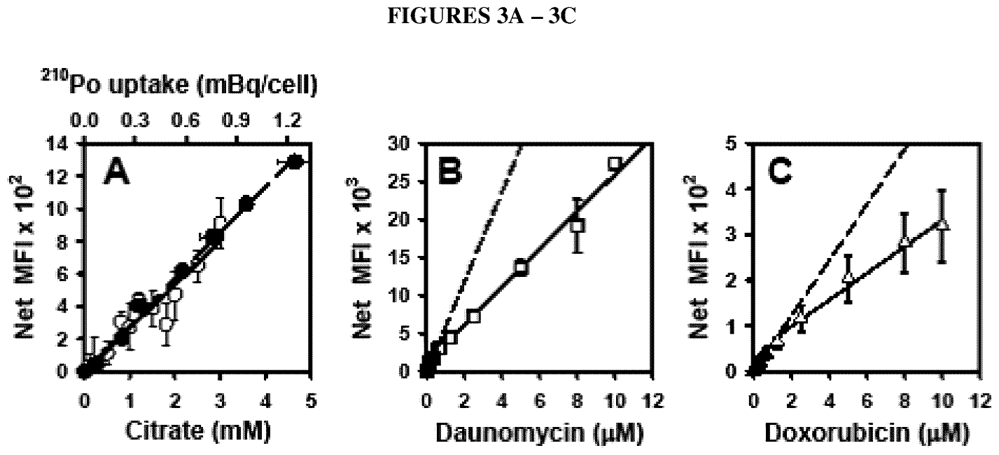

FIG. 1 shows flow cytometry histograms of the fluorescence intensity of V79 cells that were treated with ²¹⁰Po-citrate (FIG. 1A), daunomycin (FIG. 1B), or doxorubicin (FIG. 1C) at concentrations ranging from 0-3 mmol/L, 0-10 μmol/L, and 0-10 μmol/L, respectively. Note that the peaks shift toward higher mean fluorescence as the extracellular concentration of the drug increases. The relatively symmetric nature of the histograms as plotted on a linear-log scale is suggestive of a lognormal distribution of each agent among the cell population. Fluorescence intensity distribution is a lognormal function of the fluorescence intensity I,

$$f ( I ) = \frac{g}{I σ \sqrt{2 π}} {e}^{\frac{- {( lnI - {μ}_{I} )}^{2}}{2 {σ}^{2}}} , I > 0$$

where μ_{I} is the scale parameter, σ is the shape parameter, and g is a constant. Least squares fits of the data to this distribution are shown in FIG. 2. Although not observed for EuTc-citrate (FIG. 2A), there is a decrease in the breadth of the lognormal distributions corresponding to daunomycin (FIG. 2B) and doxorubicin (FIG. 2C). Treatment with 0.1 mmol/L citrate resulted in a large increase in mean fluorescence intensity (MFI) from ˜163 in untreated samples to ˜2000. This can be attributed to the high sensitivity of EuTc for detecting citrate. EuTc is capable of detecting citrate in solutions at concentrations ˜1000-fold lower. Since 0.1 mmol/L corresponded to an intracellular ²¹⁰Po activity of ˜0.02 mBq/cell, which translates to no significant cell kill, background fluorescence of 2000 units was subtracted from the MFI of each sample to obtain a net MFI. The net MFI was then plotted as a function of extracellular citrate concentration (FIG. 3A). With knowledge of the linear correlation between MFI and extracellular citrate concentration, and knowledge of the linear correlation between cellular uptake of ²¹⁰Po and extracellular ²¹⁰Po-citrate concentration, a similar correlation could be established between MFI and intracellular ²¹⁰Po activity (FIG. 3A). A very strong correlation is apparent between cellular incorporation of the vehicle citrate and intracellular ²¹⁰Po-activity. Similarly, the fluorescence histograms obtained after treatment of cells with daunomycin and doxorubicin are presented in FIGS. 3B and 3C, respectively. For both drugs, the MFI for the untreated controls were subtracted as background from the MFI of each sample, and the net MFI was plotted against extracellular drug concentration. In each case, net MFI was linearly correlated with drug concentration.

Briefly, flow cytometry was used to quantify their mean fluorescence intensity (MFI) per cell, <I>, as a function of the concentration of the agent in the cell culture medium. The net mean fluorescence intensities per cell, <I>ₙₑₜ, were determined by subtracting control autofluorescence <I>_{control}, according to the following equation:

<I>ₙₑₜ=<I>−<I>_{control}

The surviving fraction SF of cells exposed to the agent was assessed with a clonogenic survival assay and plotted as a function of several different variables including extracellular concentration, <I>ₙₑₜ, absorbed dose (Gy), and mean cellular activity (mBq/cell). The resulting survival curves were of a 1- or 2-component exponential form. Analogous to the cellular activity and absorbed dose required to achieve 37% survival, a₃₇ and D₃₇, the net mean lethal fluorescence intensity of the drug required to achieve 37% survival, <I>ₙₑₜ₃₇ , can be defined similarly and obtained from plots of SF versus <I>ₙₑₜ.

### Cellular Dosimetry

The absorbed dose to the cell nucleus was determined as known in the art. Since cells were treated with ²¹⁰Po-citrate as a single-cell suspension and were subsequently seeded for colony formation, the small contribution of cross-irradiation from neighboring cells in the colony can be ignored because it is essentially counterbalanced by the reduction in self-dose caused by flattening of cells during the colony forming period. The data was least-squares fitted to obtain a mean biologic half-time of 11.6 h. Considering the physical half-life of 138 d for ²¹⁰Po, this yields an effective half-time Tₑ of 11.6 h. This Tc, the maintenance period of 2.5 h, the subcellular distribution of ²¹⁰Po-citrate (28% nucleus, 72% cytoplasm) for V79 cells and published S values, were used to calculate a mean absorbed dose to the cell nucleus of 5.8 Gy/mBq of ²¹⁰Po incorporated into the cell.

### Toxicity of ²¹⁰Po-Citrate, Daunomycin, and Doxorubicin

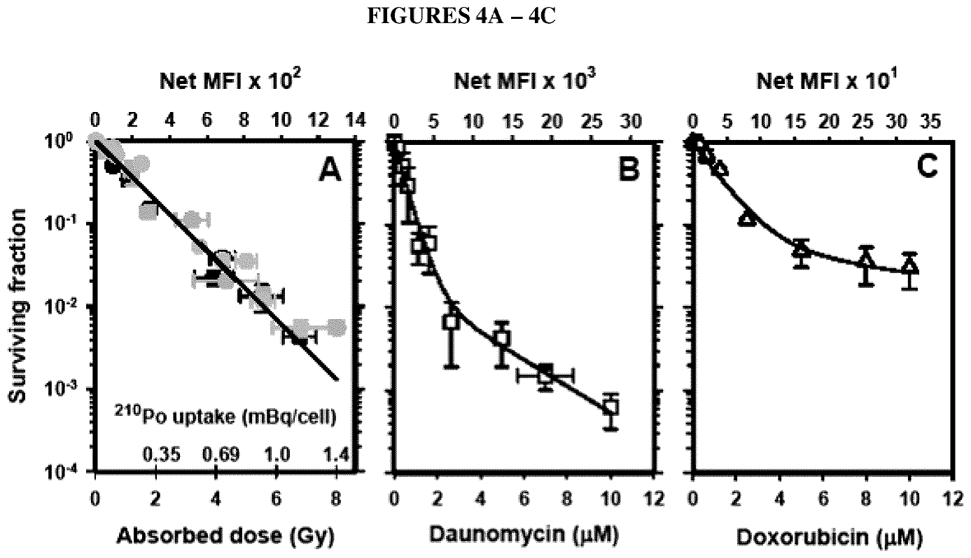

To evaluate ²¹⁰Po cytotoxicity, the surviving fraction was plotted as a function of EuTc-citrate net MFI, mean cellular uptake of ²¹⁰Po, and mean absorbed dose to the nucleus (FIG. 4A). The data indicate that net MFI of the vehicle (citrate) is a good predictor of ²¹⁰Po toxicity within the range of cellular activities employed. The relationships between cell survival and EuTc-citrate net MFI or cellular ²¹⁰Po activity can be described by an exponential function SF=exp(−A/A₁). The relationship between cell survival and drug net MFI (or extracellular concentration) for daunomycin and doxorubicin are illustrated in FIGS. 4B and 4C, respectively. For both drugs, clonogenic survival and cellular drug uptake (as determined by net MFI) are related via two component exponential functions SF=b exp(−A/A₁)+(1-b) exp(−A/A₂). A is the intracellular activity of ²¹⁰Po-citrate, absorbed dose to the cell nucleus, or drug concentration. Least squares fits of the survival data to this function were performed. The variable b is a fitted parameter.

### Role of Agent Distribution in Cellular Toxicity

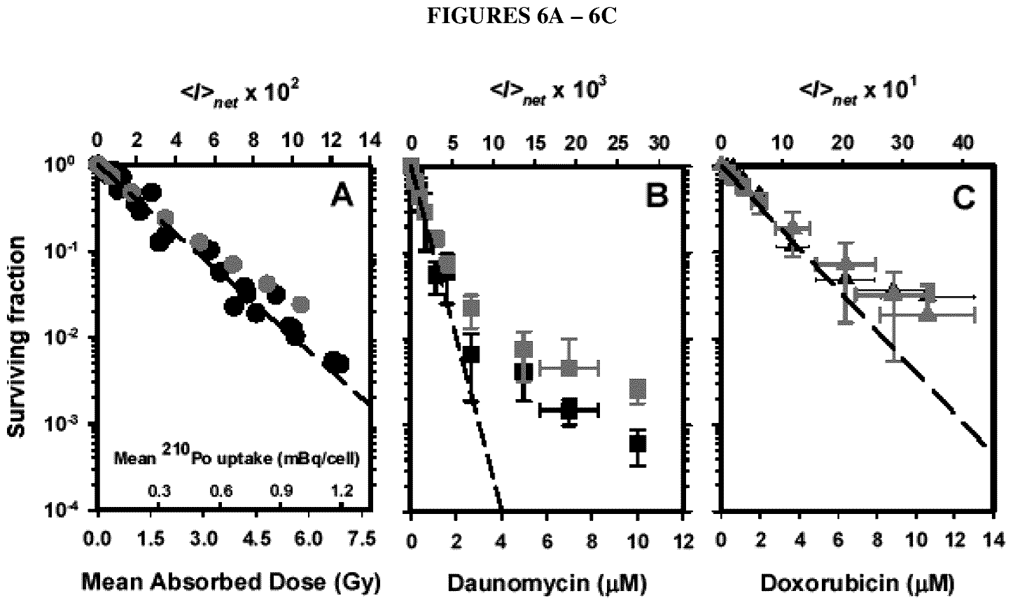

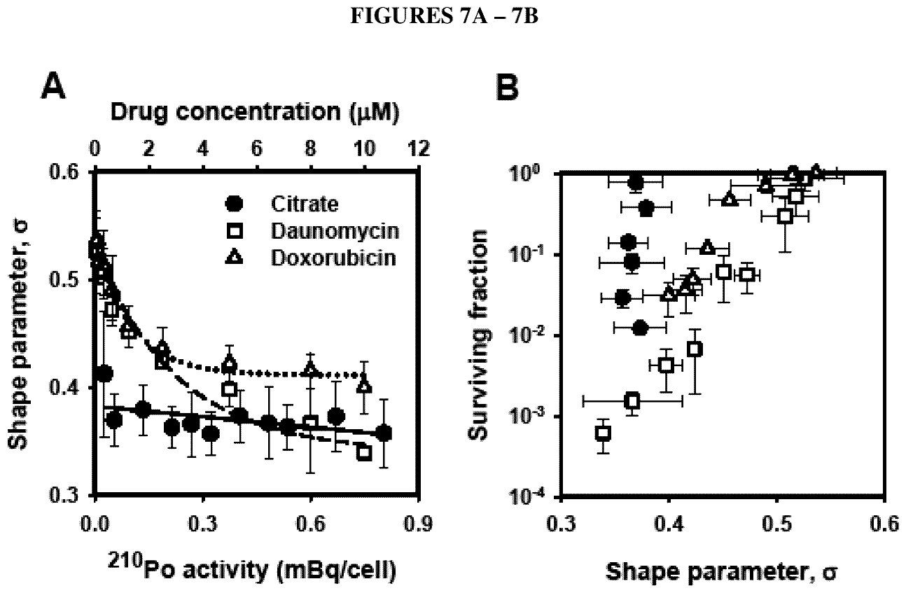

To evaluate the role of the distribution of ²¹⁰Po-citrate, daunomycin, and doxorubicin within a cell population in their subsequent toxicity, the fluorescence histograms presented in FIG. 1 were fitted to the lognormal probability density function to obtain the shape parameter σ (FIGS. 1 and 2). Although increasing intracellular ²¹⁰Po activity did not have an appreciable effect on σ over the entire range of concentrations studied for ²¹⁰Po-citrate, increases in extracellular drug concentration had a marked impact on σ for both daunomycin and doxorubicin. The relationship between cell survival and σ for the three agents is illustrated in FIGS. 6 and 7. These plots show that σ for ²¹⁰Po-citrate does not change appreciably as the surviving fraction decreases. However, σ for daunomycin and doxorubicin decreases substantially as the surviving fraction decreases.

Chemotherapy drugs and radiopharmaceuticals are typically heterogeneously distributed in tissues at the macroscopic, cellular, and subcellular levels. In the case of radiopharmaceuticals, this complicates estimation of cellular absorbed doses based on cellular activities, and causes the relationship between incorporated radioactivity and biologic response to be complex. Several in vitro studies have demonstrated saturation in cell kill with increasing activity per cell following exposure to a variety of radiochemicals, and have attributed the phenomenon to the lognormal nature of the agent distribution. This has also been shown for two chemotherapeutics, daunomycin and doxorubicin. Given the difficulty that is being experienced clinically in terms of sterilizing tumor cell populations with these and other agents, a more thorough understanding of their lognormal distributions and how they affect cell killing is needed to assist in selecting combinations of agents and guide the dosing of the constituent agents. Some enlightenment can be obtained by interpreting the flow cytometric and clonogenic survival studies described above. FIG. 1 demonstrates that flow cytometry can, under certain circumstances, be used to quantitate intracellular drug concentration. In the present case, this approach is used for EuTc-citrate (surrogate for ²¹⁰Po-citrate), and two different chemotherapy drugs, daunomycin and doxorubicin. The distributions of intracellular agent concentration are lognormal (FIG. 2). As shown in FIG. 2A and FIG. 1, the EuTc-citrate is exquisitely lognormal throughout the range of extracellular drug concentrations studied. Not only is it lognormal, but the breadth of the peak remains consistent as well. This fact is confirmed by the absence of change in σ that is observed for EuTc-citrate in FIGS. 6 and 7. In contrast, the breadths of the peaks and their corresponding σ values change markedly for daunomycin and doxorubicin (FIG. 7). Furthermore, there are notable exceptions to the lognormality of the data acquired for daunomycin and doxorubicin (FIG. 1). In the case of daunomycin, there appears to be a growing population of cells on the low fluorescence side of the peak as the extracellular concentration increases. Conversely, doxorubicin's small departure from lognormality occurs at low extracellular drug concentrations. These changes occur in concert with changes in the slope of the drug uptake versus concentration of the drug in the extracellular medium, as emphasized by the dashed versus solid lines in FIGS. 3B and 3C. The net MFI of intracellular EuTc-citrate is strongly correlated with both extracellular citrate concentration and intracellular ²¹⁰Po activity (FIG. 3A), indicating that MFI of EuTc-citrate is related to ²¹⁰Po toxicity. Similarly, the data for daunomycin and doxorubicin in FIGS. 3B-C support the notion that the extent of agent incorporation by cells can be used as a predictor of their cytotoxicity. To validate the latter, cell survival is plotted against net MFI and extracellular drug concentration, or against intracellular ²¹⁰Po activity and absorbed dose to the cell nucleus from ²¹⁰Po-citrate (FIG. 4A). For ²¹⁰Po-citrate, less than 2-logs of cell killing is observed. The relationship between the surviving fraction and net MFI (or cellular activity or absorbed dose) is exponential. Notably, neither the slope of the cellular uptake curve (FIG. 3A), nor the slope of the survival curve (FIG. 4A), nor the value of the shape parameter (FIGS. 6 and 7), change over the course of the concentrations required to achieve zero to two logs of cell kill. These conditions may be requirements needed to achieve a monoexponential survival curve and avert tailing of the survival curve. The data in FIGS. 4B-C illustrate that cell survival is related to extracellular concentration (or net MFI) of daunomycin and doxorubicin by a 2-component exponential function, with tails analogous to those observed using radiochemicals. Daunomycin and doxorubicin are closely related anthracyclines and interact with DNA by intercalation. Based on extracellular drug concentration in V79 cell cultures, daunomycin ultimately emerged to be more cytotoxic than doxorubicin. While this may be due to differences in the extent to which the drugs are incorporated, this cannot be ascertained by flow cytometry alone but rather with the help of cellular uptake studies with daunomycin and doxorubicin labeled with ³H or ¹⁴C at known specific activities. What is certain is that the slope of the cellular uptake curves (FIGS. 3B-C), and the slope of the survival curves (FIGS. 4B-C), and the value of the shape parameter (FIG. 7), all changed over the span of concentrations required to see the emergence of a tail in the survival curves. The presence of these conditions appears to be related to the two-component exponential survival curves. In fact, the concentration (ca. 1-2 μmol/L) at which these parameters begin to change (FIGS. 2, 3, 6, and 7) appears to coincide with the transition to the second component (FIG. 4).

The mean lethal concentrations for daunomycin and doxorubicin are 0.24 and 1.26 μmol/L, respectively. This indicates that low extracellular concentrations of daunomycin are ˜5×more lethal than doxorubicin in V79 cells. The mean lethal absorbed dose for ²¹⁰Po-citrate is 1.2 Gy. This arises from an uptake of 0.21 mBq/cell which corresponds to about 3600 atoms of ²¹⁰Po. Although the survival curve is similar to that obtained previously, the present mean lethal dose is higher than the former value of 0.7 Gy. This is largely due to improved S values. Although, there is an interest in using multimodal approaches that involve the concomitant delivery of chemotherapeutic and radiotherapeutic agents for cancer treatment, the efforts have mostly not been directed at using agent-specific distribution profiles to target all malignant cells. To facilitate the design of cocktails that effectively target all cells of interest, an in-depth knowledge of the distribution profile of each agent is required. This warrants the ability to express cellular incorporation of agents in absolute units on a cell-by-cell basis. As an initial step towards this end, the flow cytometric histograms presented in FIG. 2 were fitted to the lognormal probability density function, and the derived shape parameters (σ) were plotted against intracellular ²¹⁰Po activity or extracellular drug concentration. It is not surprising that these data are closely analogous to the established relationship between heterogeneity of intracellular incorporation of doxorubicin and extracellular drug concentration, as the shape parameter is a measure of the broadness of a distribution profile. While a small σ implies a narrow distribution profile (i.e., σ→0) when all cells incorporate the same amount of agent), a large σ signifies a wide spread in distribution. In practice, σ>0 and therefore subpopulations of cells will always incorporate subtoxic amounts of any given agent. However, as it has been shown as part of the present invention, the value of σ is not itself necessarily the primary determinant of the shape of the survival curve. Rather, changes in the value of σ (FIG. 7) and changes in the slope of the cellular uptake curves (FIGS. 3B and 3C), appear to correlate with changes in the transition from the first component to second component of the two-component exponential survival curves (FIG. 4B and 4C). Hence, formulation of recipes for combined modality therapy should seek to use flow cytometry distribution information to identify the drug concentration that will achieve the first component of killing. A similarly optimized additional agent could then be added with the aim of targeting cells that had low uptake of the first drug. Successively adding additional drugs would ultimately seek to achieve a net heterogeneity of σ→0, based on incorporation of all agents. It should be noted that findings related to the distribution of therapeutic agents among a population of cells, and their corresponding dose response characteristics, may vary considerably depending on cell type and the microenviroment within which the cells reside. In addition, factors such as resistant subpopulations can have a significant impact on the shape of the response curve. Therefore, caution is needed when cocktails are formulated based on in vitro findings and then extrapolated to the in vivo setting encountered in the clinic.

The present invention demonstrates that the distribution of cellular radioactivity within a cell population is adequately described by a lognormal probability density function. The ubiquitousness of the lognormal distribution has been further demonstrated by the cellular uptake profiles of two different chemotherapeutic drugs. Changes in the value of the lognormal shape parameter and changes in the slope of the cellular uptake curves with increasing drug concentration flag the onset of saturation in the dose response curve. Accordingly, measurement of these changes using flow cytometry, or another analytical technique, preferably a high-speed technique, can be employed to rapidly predict biological response to the drug, and ultimately to formulate a highly effective therapeutic cocktail.

### Cocktails of Therapeutic Agents

One multiple-therapeutic agent embodiment of the present invention involves a cocktail of radioimmunotherapy and chemotherapy agents. Informed radioimmunotherapy/chemotherapy is one option for front-line defense against metastatic and residual disease in adjuvant external beam radiotherapy and surgery. However, one major limitation has been the difficulty in relating cellular incorporation of therapeutic agents, on a cell-by-cell basis, to resulting biological effects. The models for predicting the distribution of cytotoxic agents among a cell population, at the single-cell level, as disclosed above, now also provide a cocktail approach whereby all malignant cells can be effectively targeted. This flow cytometry-based approach, taking explicit account of the lognormal distribution of cellular uptake of the agents, enables prediction of treatment response on a cell-by-cell basis, and has now been shown to be invaluable in the selection of agents for the design of highly effective therapeutic cocktails that are capable of targeting the diversity in tumor cell populations. Further, such cocktails can be created not only for treatment of cancer, but also for infectious diseases, and other diseases that are amenable to targeted therapies. Furthermore, this single-cell Monte Carlo technique can be used to resolve difficulties encountered when attempting to predict biological response at the multicellular level using macroscopic mean agent doses.

Over the past two decades, interest in the use of -emitting radionuclides in radio-immunotherapy has grown significantly. However, a major unresolved concern is that the toxicity of α-emitting radionuclides does not allow administration of high activities. As such, targeting procedures would need to be optimized to minimize normal tissue toxicity. This can be achieved via multi-modality radioimmunotherapy, which employs combinations of radioimmunotherapy and chemotherapy. Multi-modality radioimmunotherapy approaches seek not only to effectively target all malignant cells, but also to significantly reduce the amount of each constituent of the cocktail. To guide design of effective cocktails of α-emitting radiopharmaceuticals and chemotherapy drugs, there is the need to assess the role of nonuniform agent distribution on modification of α-particle radiotoxicity by chemotherapy drugs. Furthermore, the capacity to predict such modifications in treatment response on a cell-by-cell basis should greatly improve treatment outcomes through individualized staging prior to therapy.

As has now been demonstrated, concomitant treatment of Chinese hamster V79 cells with an α-emitting radiochemical, ²¹⁰Po-citrate, and either daunomycin or doxorubicin, resulted in an enhancement of α-particle radiotoxicity. Further, the toxicity of the combination treatment can be predicted with a Monte Carlo simulation approach based only on knowledge of the initial slope of the cell survival curves of the individual agents and information on the distribution of agent incorporation among cell populations.

### Methods and Systems For Determining The Distribution of Radiation Dose and Response

The present invention further includes the previously described methods that can be further included a system, method, and computer program product for clinical treatment planning for patients in need of nuclear medicine procedures, as well as chemotherapy procedures to treat cancer, and/or for diagnostic purposes. The present invention also allows users to visualize and understand the impact of radionuclide choice, distribution of activity (cross-dose) in and among cells (disease and normal), cell dimensions, inter-cell distances, cluster size, and radiobiological response parameters on the capacity to kill populations of cells. The data used to populate may be laboratory or patient data, and one with ordinary skill in the art may create additional features and/or incorporate patient data accordingly. Embodiments of the invention may include ten or more radiopharmaceutical and/or chemotherapy/biologic agents that can be simultaneously analyzed, by incorporating the methods previously described.

This disclosure is not limited to the particular systems, methodologies or protocols described, as these may vary. The terminology used in this description is for the purpose of describing the particular versions or embodiments only, and is not intended to limit the scope.

The following terms shall have, for purposes of this application, the respective meanings set forth below:

A “computing device” refers to a device that includes a processor and tangible, computing-readable memory. The memory may contain programming instructions that, when executed by the processor, cause the computing device to perform one or more operations according to the programming instructions. Examples of computing devices include personal computers, servers, mainframes, gaming systems, televisions, and portable electronic devices such as smartphones, personal digital assistants, cameras, tablet computers, laptop computers, media players and the like. When used in the claims, reference to “a computing device” may include a single device, or it may refer to any number of devices having one or more processors that communicate with each other and share data and/or instructions to perform the claimed steps.

An “application” refers to a software program that is configured to operate on a computing device. An application may assist a user in accessing network resources from a user's computing device, e.g., a mobile application may be linked to a database to receive parameters concerning a tissue sample from a patient.

A “mobile device” refers to a portable computing device. Examples of mobile devices include mobile phones, smartphones, personal digital assistants (PDA), tablet computers, e-readers or e-books, netbooks, notebook computers, and the like. A mobile device may include one or more input devices such as a keypad, a touch-pad, a track-pad, and a touch-sensitive component that is integrated within a display, such as a captive, resistive or other type of touch screen. A mobile computing device may be configured to access a communication network via a wired or wireless connection.

A “feature” refers to a unit of software, or a distinct section of a user interface, that displays a category or related group of information associated with at least a portion of an application.

An “option” refers to a visual feature of a graphical user interface. An option may be a button, a radio dial, a drop-down menu, a hyperlink, an icon, an image, a text box, a text field and/or the like.

The term “derive(d)” is used interchangeably with the term “calculate,” and is generally used in reference to the calculation of a feature.

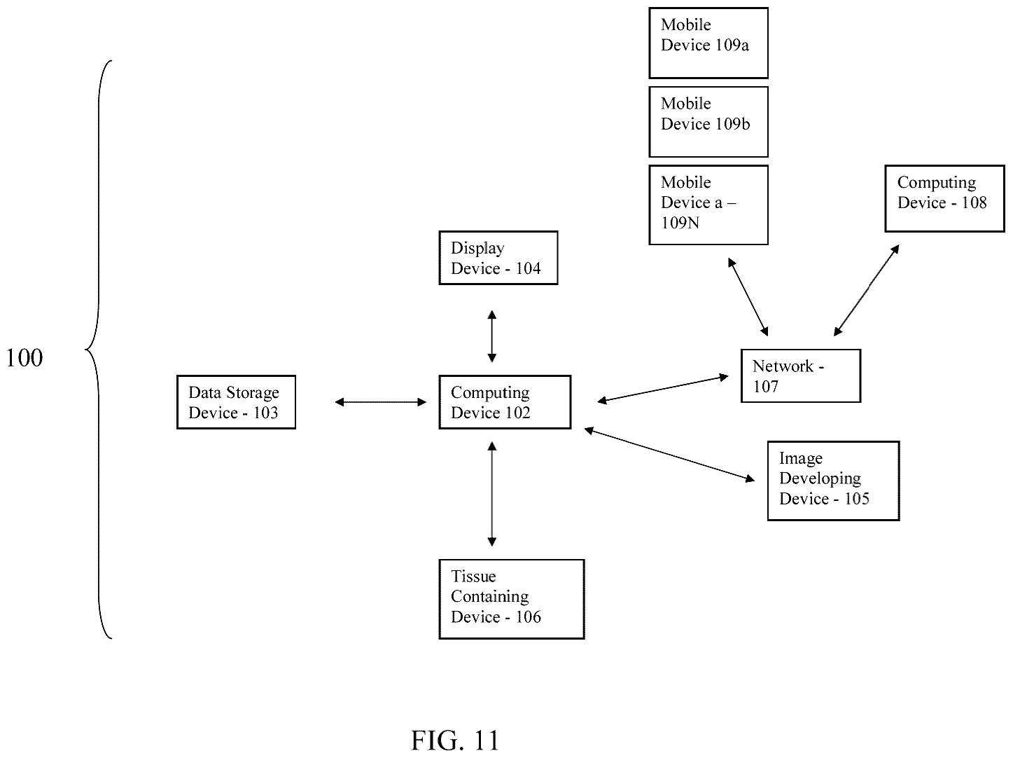

FIG. 11 illustrates a block diagram of a system 100 for clinical treatment planning of a dose of radiation in accordance with the present invention. As illustrated by FIG. 11, the system 100 may include a computing device 102, a data storage device 103, a display device 104, an image developing device 105, and a device to contain tissue from a patient 106. A device to contain tissue from a patient 106 may provide certain parameters regarding the tissue, the tissue may be in the form of a histology slide with tissue, and the device may provide information regarding e.g., the type of cells and/or other parameters that would characterize the tissue sample. Examples of other devices that may acquire information from the tissue sample are flow cytometry, various types of mass spectroscopy devices, cDNA arrays, etc. A computing device 102 may be a computing device used to create an application as described herein. A computing device 102 may be in communication with a mobile device 109a-N and/or another computing device 108 via a communication network 107. In various embodiments, the communication network 107 may be a local area network (LAN), a wide area network (WAN), a mobile or cellular communication network, an extranet, an intranet, the Internet and/or the like. In an embodiment, the communication network 107 may provide communication capability between a computing device 102 and another computing device 108 or a mobile device 109a-N. Although FIG. 11 illustrates a single computing device 102, it is understood that the system 100 may include additional computing devices 102 within the scope of this disclosure. For clarity and brevity, in FIG. 11 a single system for determining a dose of radiation 100 is shown, but it should be understood that any number of systems for determining a dose of radiation may be accessed or connected by a communication network with which to perform methods in accordance with the invention.

The system 100 may include a computing device 102 configured to operate an application for determining a dose of radiation for a patient. The determination of the dose of radiation is made in accordance with configuration data and/or patient data.

In an embodiment, a computing device 102 or 108 may create an application based, at least in part, on information it receives from a computing device 102. The computing device 102 or 108 may make an application available to one or more mobile devices 109a-N. In an embodiment, a computing device 102 may be in communication with a mobile device 109a-N via a communication network 107.

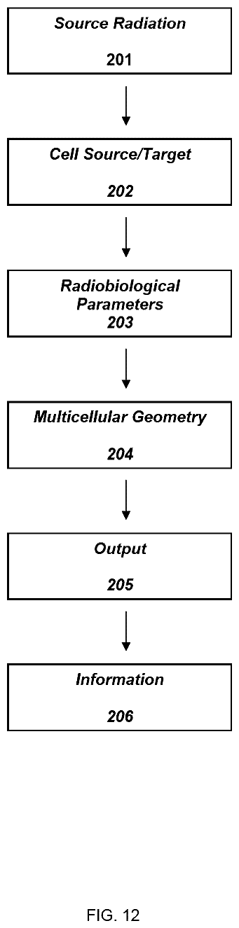

FIG. 12 illustrates a flow chart of an example method of determining a dose of radiation according to an embodiment by a computing device and/or an application of a computing device or a mobile device.

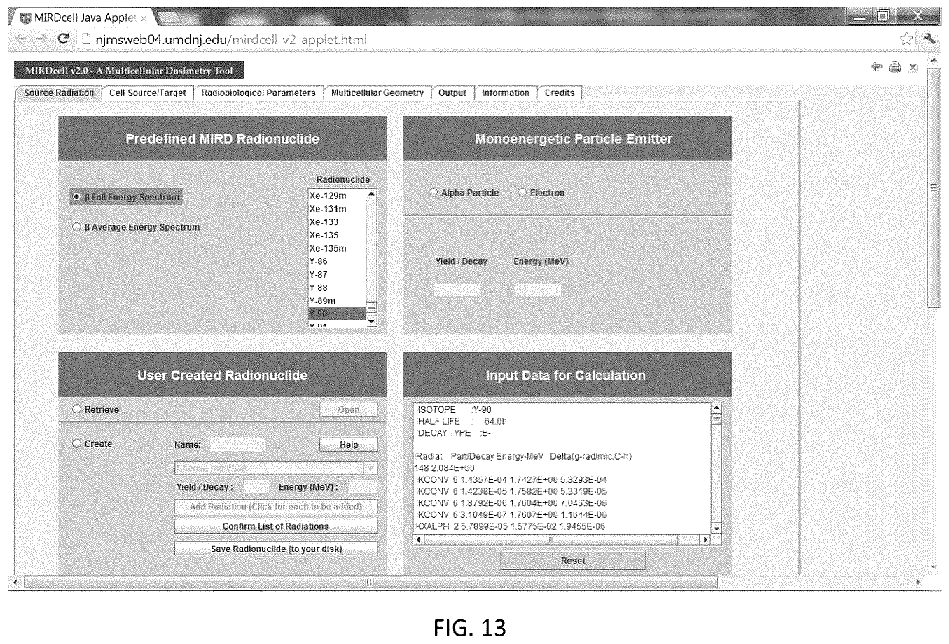

FIG. 13 illustrates a GUI for a Source Radiation 201 feature. This feature allows a user to select the radioactivity in the source cells (i.e., cells labeled with radioactivity). Upon making a selection, the radiation data may be shown in a field such as the box entitled Input Data for Calculation. In an embodiment as shown in Table 1, two or more choices may be available. The choices may include: Predefined MIRD radionuclide, Monoenergetic Particle Emitter and user-defined radionuclide. This feature may also include the background dose for low-LET or high-LET radiation. In a further embodiment, this feature or a new similar feature may include a time-factor in dose response function, account for different radiation types and /or provide distribution of biologically effective doses (BED) and equieffective doses (EQD2).

In other embodiments, this feature or a new feature can include other types of agents, such as chemotherapeutic and/or biologic drugs to treat a condition such as cancer or an infection, and may further include parameters for physical half-life, biological half times, pharmacokinetic and/or phamaco-dynamic data regarding the agents.

In an embodiment, there may be two or more options within the Predefined MIRD Radionuclide. The “B Full Energy Spectrum” option provides a dropdown list of the radionuclides for which data are provided in the MIRD Radionuclide Decay Scheme monograph. In an embodiment, data sets correspond to the radiation data from which the monograph was prepared; however, the yield and mean energies for all beta particle (β−) and positron (β+) emitters maybe replaced with full logarithmically binned β spectra. Use of the continuous β spectrum as opposed to the mean β energy can play an important role in cellular dosimetry. This altered data set was originally created for calculating cellular S values in the MIRD Cellular S Values monograph. Some of the spectra contained in excess of one thousand different radiations for a given radionuclide, many of which are insignificant with respect to internal dosimetry, in certain embodiments, radiations which contributed greater than 0.1% to the total energy emitted per nuclear transformation (Δ) for that particular radiation type may be retained. Recoil energy of the residual daughter following alpha decay may not be included because experiments indicate that this energy is not biologically relevant. The “β Average Energy Spectrum” option provides the radiation data contained on the compact disk that accompanies the MIRD Radionuclide Decay Scheme 2nd Edition. In certain embodiments, average β particle energies may be contained within this data set.

In regards to radionuclides that are part of a decay chain (e.g., ²¹¹At, ²¹³Bi, ²²³Ra, ²²⁵Ac) parent and daughter radionuclides may not be in equilibrium due to differences in biokinetics behavior. In certain embodiments, users can create files that include daughters provided that branching ratios are accounted for and should be done when the parent and daughter radionuclides are in equilibrium.

The “Monoenergetic Particle Emitter” option allows a user to select a hypothetical monoenergetic electron or alpha particle emitter. Particle energy and yield per nuclear transformation may be specified by a user.

The “User Created Radionuclide” option provides maximum flexibility by allowing the user to create a radionuclide that is not available in the predefined dropdown lists. After specifying the name of the radionuclide, the user may choose a radiation type, its yield and energy, and then selects “Add Radiation.” This process is repeated until all desired radiations have been added. “Confirm List of Radiations” is then selected. If it is desired to save the radionuclide for later use, “Save” may be selected and it may be saved to a local storage location.

Finally, once the user selects the desired radionuclide from one of the three exemplified options above, the radiation data is calculated and exemplified in the box entitled Input Data for Calculation. Specified in descending order are the radionuclide, physical half-life, and principal decay type. This is followed by the radiation data for the radionuclide; these include the total number of radiations in the file and the radiation type, yield, energy, and mean energy emitted per nuclear transition for each radiation Ai.

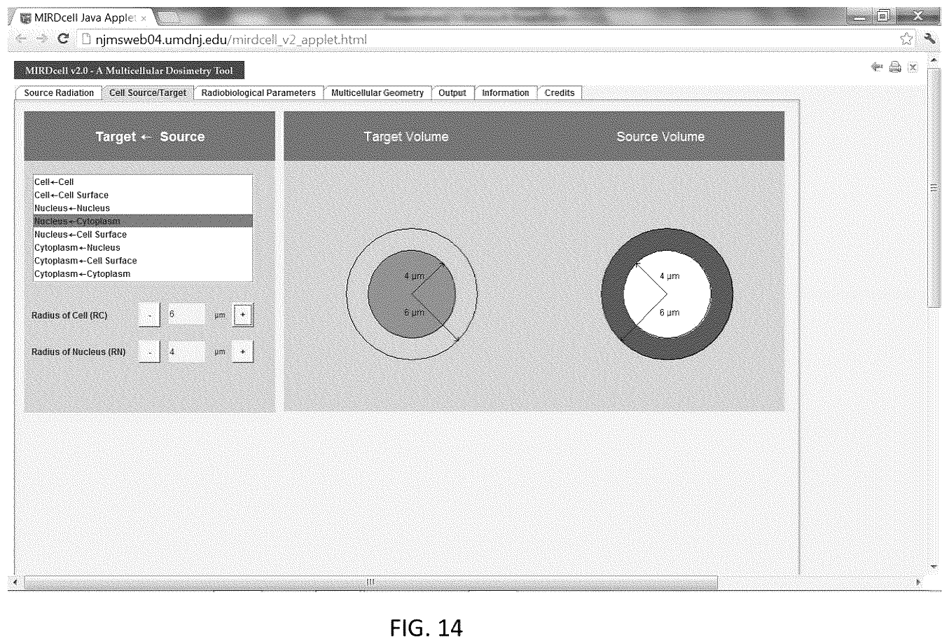

FIG. 14 illustrates a GUI for a Cell Source/Target feature 202. This feature allows a user to select source volume in cell where radioactivity is distributed. Select target volume in the cell for which radiation absorbed dose will be calculated. Select radius of cell and nucleus. The cell is modeled as two concentric spheres with radii corresponding to those for the nucleus (R_{N}) and cell (R_{C}), respectively, however additional models that take into account the cell membrane or other regions of a cell can be implemented. The cells are assumed to be composed of liquid water of unit density. The radioactivity in the cell is assumed to be uniformly distributed in the source region of the cell; selectable among cell (C), cell nucleus (N), cytoplasm (Cy), or cell surface (CS). In certain embodiments, the target region in the cell for which the radiation absorbed dose will be calculated, can be selected for example, as the entire cell, the cell nucleus and/or the cell membrane. The radii of the cell and cell nucleus may be limited to integer values and are specified by a user.

In another embodiment, the Cell Source/Target feature may contain or import patient or laboratory data regarding the target cells or diseased tissue of a patient. The patient or laboratory data may be imported through a network. This feature or an additional feature may also take into consideration the bystander effect.

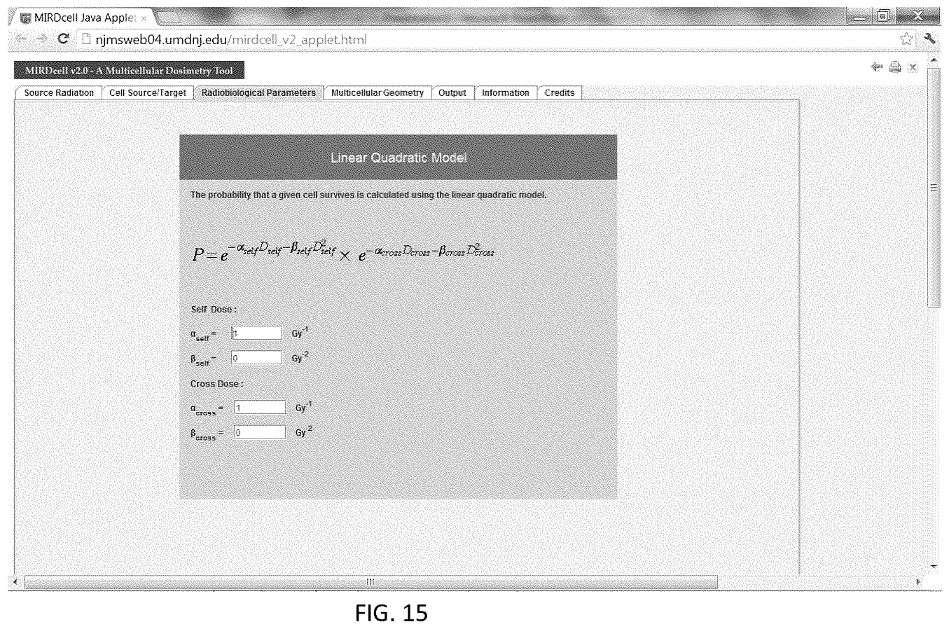

FIG. 15 illustrates a GUI for a Radiobiological Parameters feature 203. This feature allows a user to model the surviving fraction of cells in a specified cell population based on the calculated absorbed doses to the individual cells (see subsections that follow). The probability P that a given cell survives is calculated with the linear quadratic model below,

P=e^{-α}^{_{self}} ^{D}^{_{self}}^{-β}^{_{self}}^{d}^{_{self}}^{²}×e^{-α}^{_{cross}}^{D}^{_{cross}}^{-β}^{_{cross}}^{D}^{_{cross}}^{²}

Where α_{self} and β_{self} are the linear-quadratic parameters that characterize the cellular response to self-dose (D_{self}) and α_{cross} and β_{cross} characterize the cellular response to cross-dose (D_{cross}). This distinction can often be necessary for Auger electron emitters, or even beta particle emitters, when they are incorporated into the DNA. Under these circumstances, the relative biological effectiveness of Auger emitters can be akin to alpha particles. The default parameters are set to a_{self}=α_{cross}=1 Gy⁻¹, and β_{self}=β_{cross}=0 Gy⁻². These default values are arbitrary and a user may use values that are relevant to their use of the application. The Monte Carlo method can be used to determine whether a given cell survives. This feature may also enable assessment of the surviving fraction of a population of small multicellular clusters. This additional embodiment may allow a user to select the number of clusters and distribution of cluster sizes. This feature or an additional feature may take into consideration the bystander effect.

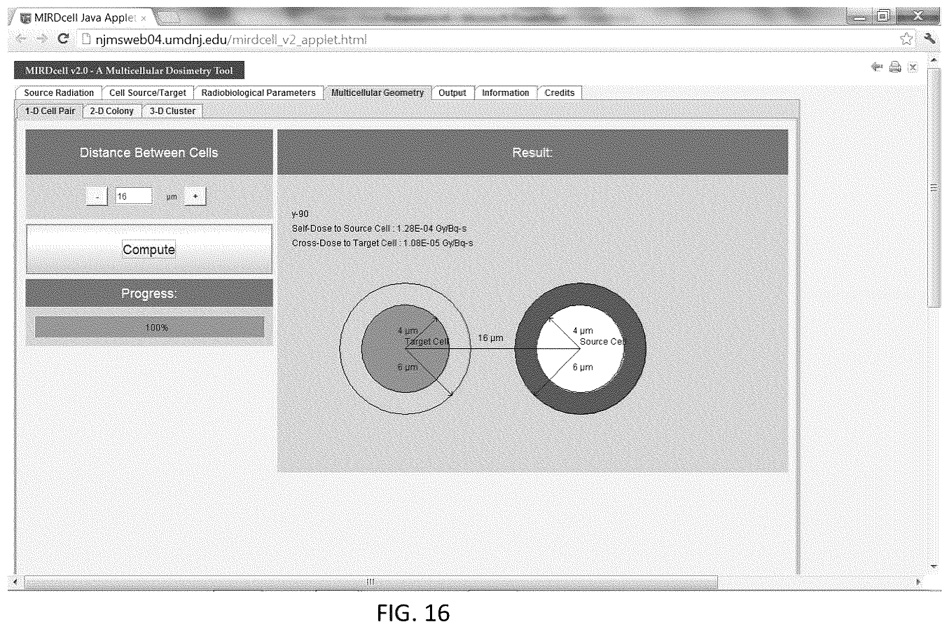

FIG. 16 illustrates a GUI for a Multicellular Geometry feature 204. Within the Multicellular Geometry feature, a user may input the activity distribution, or select the activity distribution provided by a computing device, a data storage device, an image developing device or a tissue containing device. Multicellular Geometry <1-D cell Pair sub-feature. FIG. 16 depicts the cell geometry that is used to calculate the self- and cross-doses for a pair of cells. This configuration is considered 1-dimensional (1-D). The source volume that contains radioactivity is generally defined by a color such as the color red. The target volume in the neighboring cell is also generally defined by a color such as the color blue. The user may set the distance (d) between the centers of two cells that are nearest neighbors. The self-dose and cross-dose S values (mean absorbed dose per unit cumulated activity in the source region) for the specified source and target regions are calculated upon selecting a Compute button. The self-dose and cross-dose S values are calculated using stopping powers and geometric factors (J Nucl Med. 1994; 35:303-316 and J Nucl Med. 1994;35:521-530). As in the MIRD Cellular S value monograph, the stopping power relationship of Cole may be used for electrons and alphα-particle stopping powers from reports of the International Commission on Radiation Units and Measurements. Also, Also, Monte Carlo radiation transport codes are available for low-energy Auger electron emitters such as ¹²⁵I localized on the cell surface or cytoplasm. In principle, stopping power data from other literature could be used. In addition, stopping power data for media other than water could also be used.

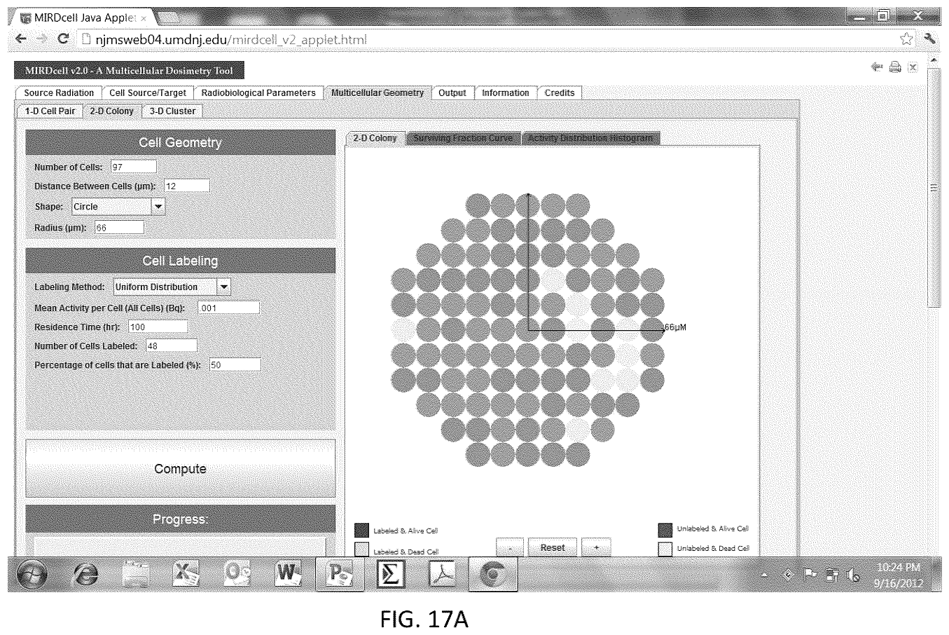

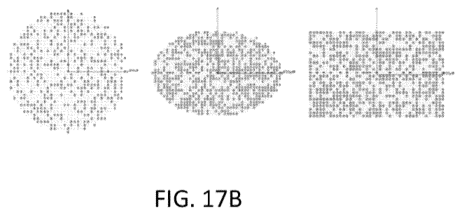

FIG. 17A illustrates a GUI for a Multicellular Geometry <2-D Colony<2-D Colony, another sub-feature, within the Multicellular Geometry feature. This sub-feature is used to create a cell population that resides on a plane (i.e., colony). It is preferable that the application contain a Cell Geometry box, to allow a user to specify the number of cells in the colony, distance between the cells, and the shape and dimensions of the colony (circle, rectangle, ellipse, as shown in FIG. 17B). Alternatively, the shape can be selected first and then the dimensions of the colony can be specified. It is preferable that the application contain a Cell Labeling box, a user may select the distribution of activity among the labeled cell population (uniform, normal, lognormal) and enter the standard deviation of the mean for the normal distribution or shape factor for the lognormal distribution. A uniform activity distribution among the labeled cells implies that each labeled cell has the same initial activity A in its source region. In the normal distribution, the initial activity per cell is distributed according to the probability density function:

$$f ( A ) = \frac{1}{A σ \sqrt{2 π}} {e}^{\frac{- {( A - 〈 A 〉 )}^{2}}{2 {σ}^{2}}}$$

Where <A> is the mean initial activity per cell and σ is the standard deviation of the mean. It is preferable that a user avoid entering standard deviations that can result in negative values of A. When this occurs, the user is prompted to choose a smaller value for σ. In the case of the lognormal distribution, the activity per cell is distributed according to the probability density function:

$$f ( A ) = \frac{1}{A σ \sqrt{2 π}} {e}^{\frac{- {( lnA - ( ln 〈 A 〉 - {σ}^{2} / 2 ) )}^{2}}{2 {σ}^{2}}} , A > 0$$

where σ is the lognormal shape parameter. Thus, if <A> is known experimentally, then only σ is required. Mean Activity per Cell (labeled+unlabeled) and time-integrated activity coefficient (ã) are then entered. The time integrated activity coefficient (also known as the cumulated activity) is defined in MIRD Pamphlet No. 21. A user may then specify either the Number of Cells Labeled with radioactivity or the Percentage of cells that are Labeled. Once all the parameters are specified the user instructs the computing device to calculate the inputted variables to derive a Multicellular Geometry and its biological response. Calculation times may vary dramatically depending on the number of radiations emitted by the selected radionuclide, the range of the particles emitted, and the percentage of cells that are labeled. A progress bar appears below the Compute button to provide the status of the calculation. Progress for Part 1 corresponds to the calculation of all necessary self- and cross-dose S values. Progress for Part 2 corresponds to the process of creating a virtual assembly of cells in a Cartesian coordinate system with a close packed square lattice (number of cells is displayed), assigning activity to each cell, tallying self- and cross-doses for each cell, calculating the surviving fraction of cells (see below), and plotting the colony geometry in the graphical user interface. Labeled cells are selected randomly and each cell randomly assigned an initial activity according to user-selected distribution. The time integrated activity in the source compartment of each cell is calculated by taking the product of the initial activity in the cell and the user-specified time-integrated activity coefficient (a). The activity in all labeled cells is assumed to have the same residence time.

In another embodiment, the activity distribution can be predicted based on input from devices that analyze patient samples. For example, as described in the literature (J. M. Akudugu and R. W. Howell, A method to predict response of cell populations to cocktails of chemotherapeutics and radiopharmaceuticals: Validation with daunomycin, doxorubicin, and the alpha particle emitter ²¹⁰Po. Nucl Med Biol 39, 954-961 (2012)), flow cytometry can be used to determine the distribution of drug uptake of one or more drugs among the cell population. The methods previously described to determine to predict an individual patient's cells may be incorporated into this or an additional related feature. Each of these drugs can be labeled with radioactivity so the activity distribution for each is known. Each of these drugs can be labeled with radioactivity so the activity distribution for each is known. These data can then be used to calculate the self- and cross-dose to each cell in the population analogous to the description above. High throughput methods that quantify drug targets on a cell-by-cell basis can also be used to provide input information. Finally, in another embodiment, both nonradioactive drugs and radioactive drugs can be analyzed to obtain the optimum therapeutic cocktail.

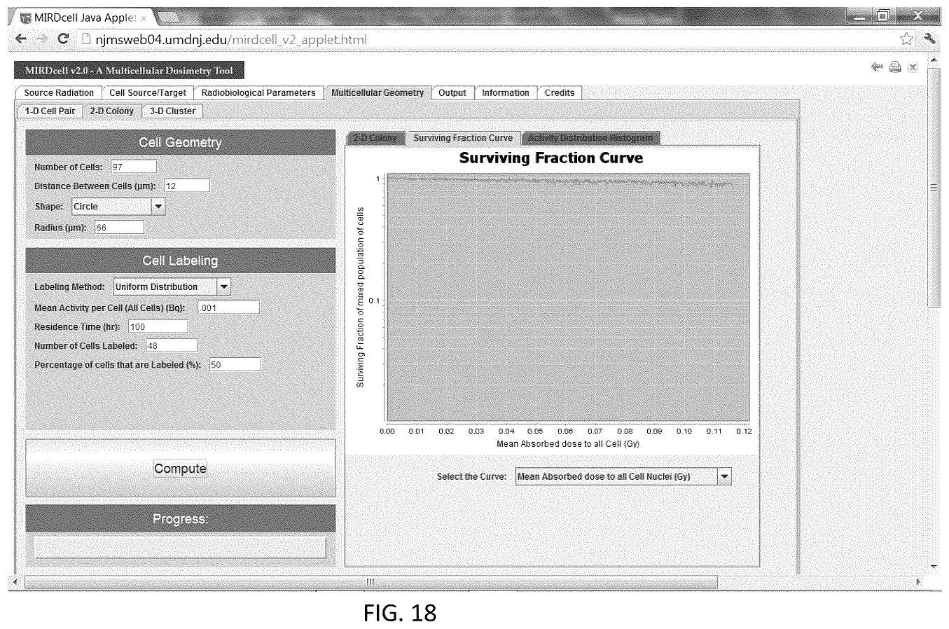

FIG. 18 illustrates a GUI for a Multicellular Geometry <2-D Colony<Surviving Fraction sub-feature within the Multicellular Geometry feature. A user may instruct the computing device to calculate and derive the surviving fraction of cells in the colony. The surviving fraction is calculated using the Monte Carlo method. For each cell, a survival probability is calculated by substituting its self- and cross-dose into the survival probability equation. A random number between 0.0 and 1.0, which may be generated with Java method Math.random( ) is compared with the survival probability. If the random number was smaller than the generated probability, the cell was scored as a survivor. Otherwise, it is scored as dead (i.e., having undergone reproductive failure). The fraction of survivors among the cell population that compose a given simulation represents the surviving fraction of the cell population. This process is repeated for numerous values of <A> up to a maximum value corresponding to the user assigned mean activity per cell. The resulting surviving fractions are plotted as a function of cellular activity or absorbed dose to the labeled, unlabeled, and entire cell population according to user-selectable ordinates and abscissae. These choices allow the user to explore characteristics of the response of each population of cells. In general labeled cells receive both self- and cross-doses, whereas unlabeled cells receive only cross-dose.

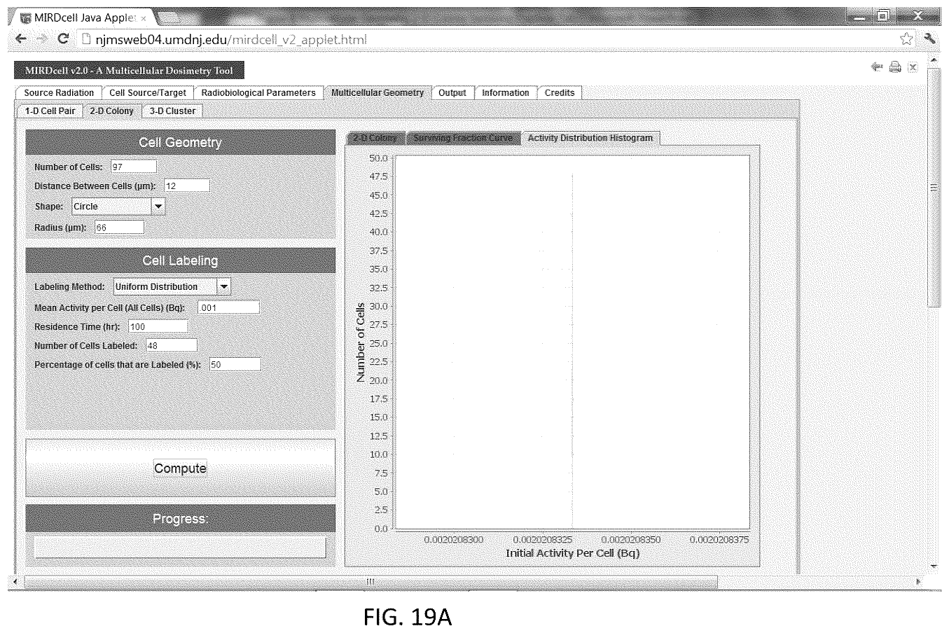

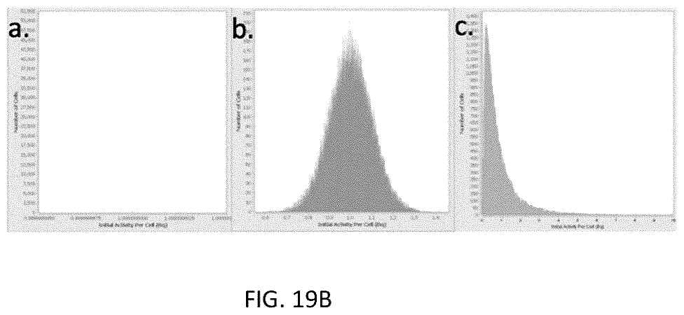

FIG. 19A illustrates a GUI for a Multicellular Geometry <2-D Colony<Activity Distribution Histogram sub-feature. The user-specified activity distribution is plotted in this feature. Data for this feature can be imported from laboratory or patient data.

FIG. 19B illustrates examples of uniform, normal and lognormal distributions. Initial activity histogram for labeled cells: a) Uniform Distribution, b) Normal Distribution with mean activity per cell equal to 1 Bq and standard deviation equal to 0.1 Bq , c) Lognormal Distribution with mean activity per cell equal to 1 Bq and shape factor equal to 1. These distributions are also available in the 3-D Cluster.

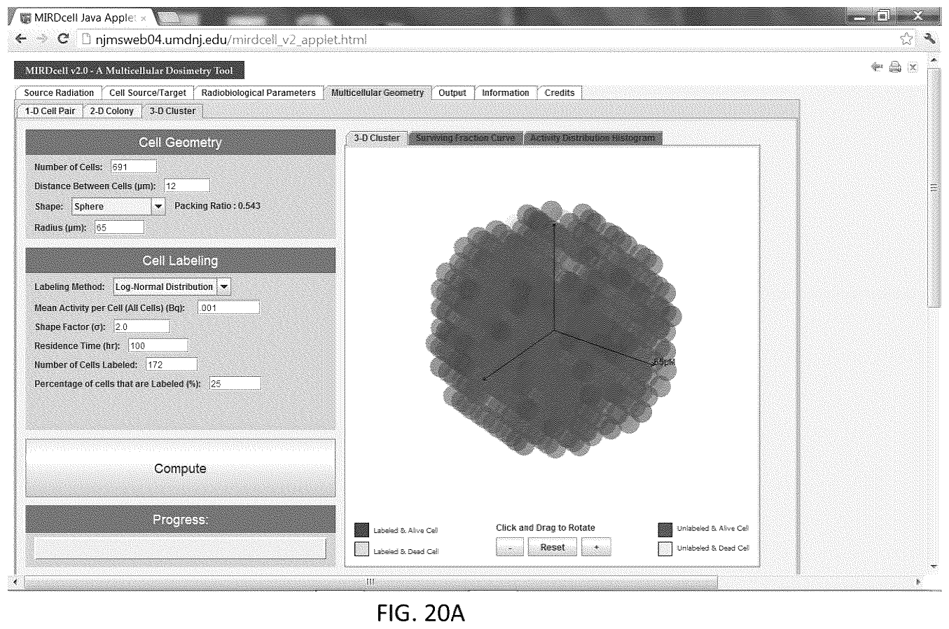

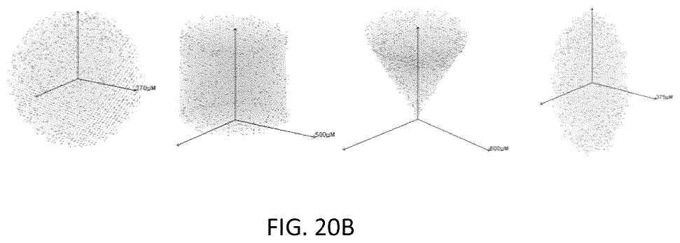

FIG. 20A illustrates a GUI for a Multicellular Geometry <3-D Cluster<3-D Cluster sub-feature. The planar cell configuration described above may be extended to three dimensional clusters as shown in FIG. 20A. The user may select the shape of the cluster to be a sphere, ellipsoid, rod, or cone, or other shapes that a user may define for an application (FIG. 20B). The desired dimensions of the cluster are then entered along with the time-integrated activity coefficient (ã), mean activity per cell (labeled+unlabeled cells), and number or percentage of cells labeled. The user instructs the computing device to calculate and derive a Multicellular Geometry <3-D Cluster e.g., by selecting for example, a Compute button, the simulation and modeling begin. The process is essentially the same as for the 2-D Colony except that the cluster is assembled in a three-dimensional Cartesian coordinate system in a close-packed cubic lattice. Other embodiments could use different cell packing configurations (e.g., hexagonal close packed). Varying cell diameters and shapes could be supported in yet another embodiment, and data for this feature can be imported from laboratory or patient data.

FIG. 20B illustrates examples of additional geometries available in Multicellular Geometry <3-D Cluster<3-D Cluster. These are 3-D Cluster shapes containing 10000 cells, with 50% of the cells labeled with Y-90, a user may define the amount of cells per cluster, several to in excess of 100,000 cells. Cluster shapes are Sphere, Rod, Cone, and Ellipsoid. Cells are labeled (red/dark grey) or unlabeled (green/light grey), and the transparency represents whether the cell is dead (transparent) or alive (opaque).

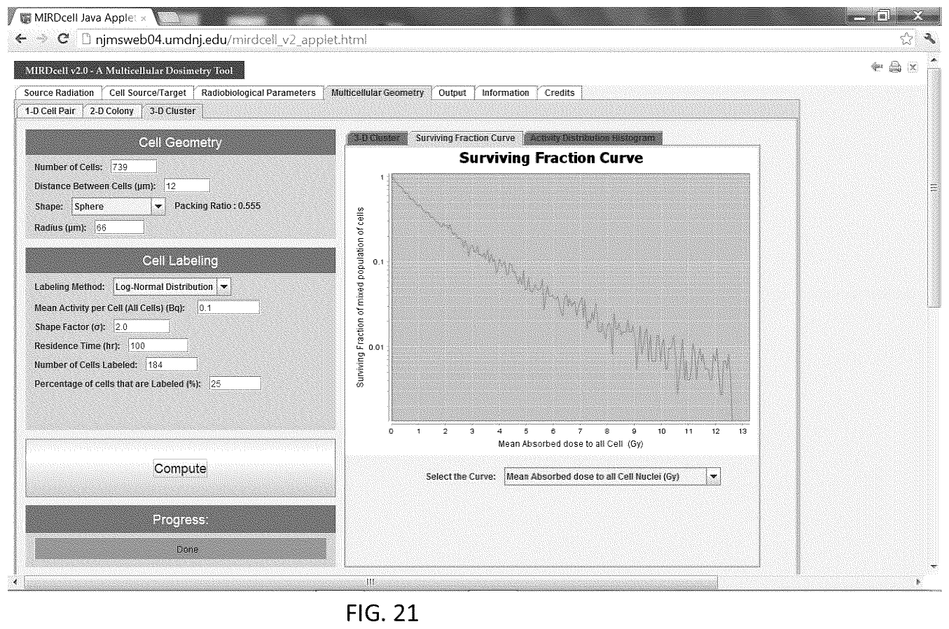

FIG. 21 illustrates a GUI for a Multicellular Geometry <3-D Cluster <Surviving Fraction sub-feature (FIG. 21). As in the 2-D colony case, a user may instruct the computing device to calculate and derive a Multicellular Geometry <3-D Cluster <Surviving Fraction e.g., also calculated upon selecting the Compute button.

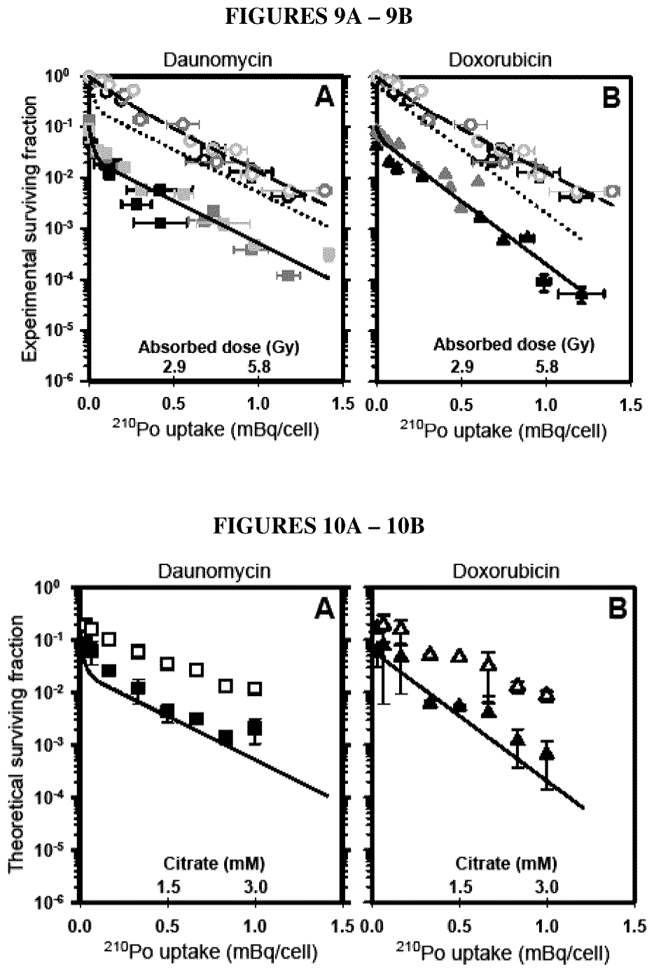

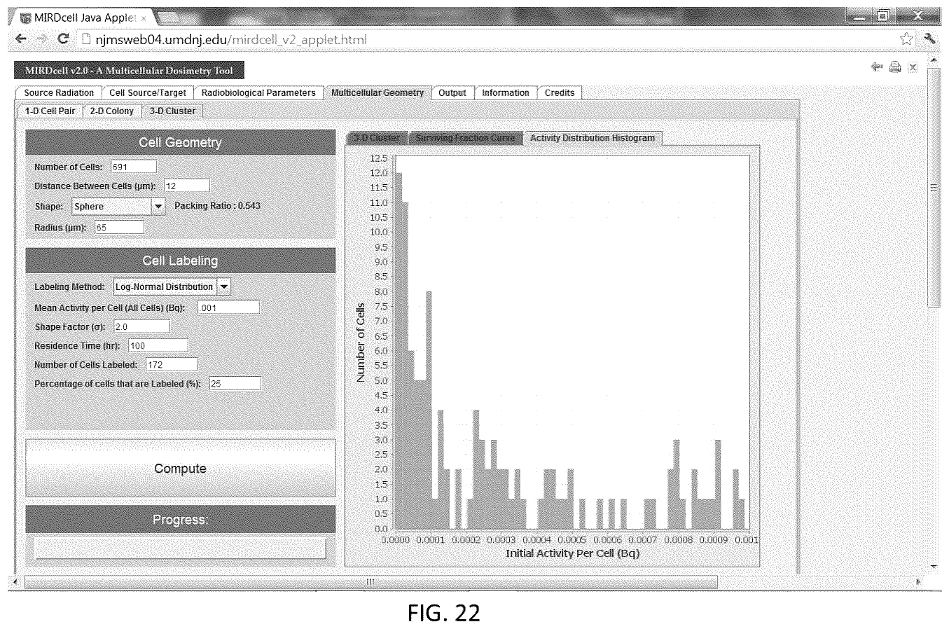

FIG. 22 illustrates a GUI for a Multicellular Geometry <3-D Cluster <Activity Distribution Histogram category. As in the 2-D colony case, the user-specified activity distribution is plotted in this sub-feature. Examples of uniform, normal and lognormal distributions are shown in FIG. 9B.

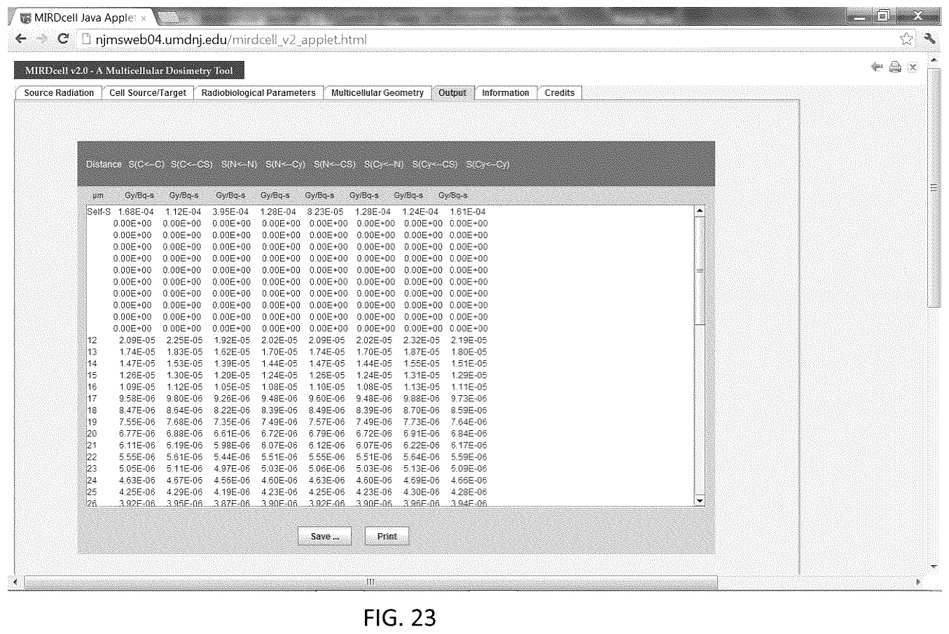

FIG. 23 illustrates a GUI for an Output feature 205. Upon completion of a calculation, a tabulation of the self- and cross-dose S values may be written to the text box within the Output feature. Other embodiments could provide the self- and cross-doses for each cell and their mean values, along with the surviving fraction. These results could be provided as tables and/or graphs including dose volume histograms and other formats suitable for the clinical environment.

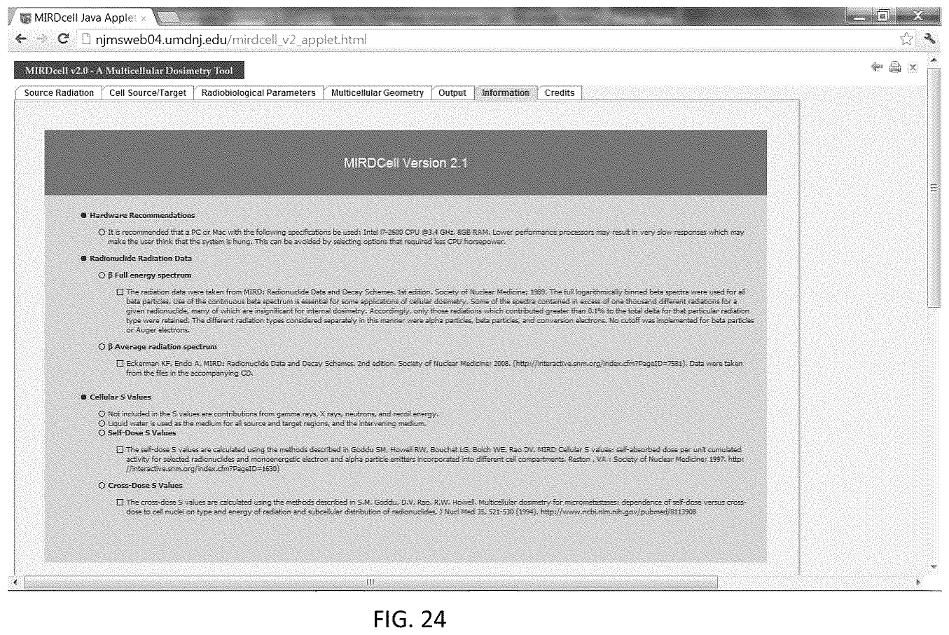

FIG. 24 illustrates a GUI for an Information feature 206. This information feature presents an abbreviated version of key information described above.

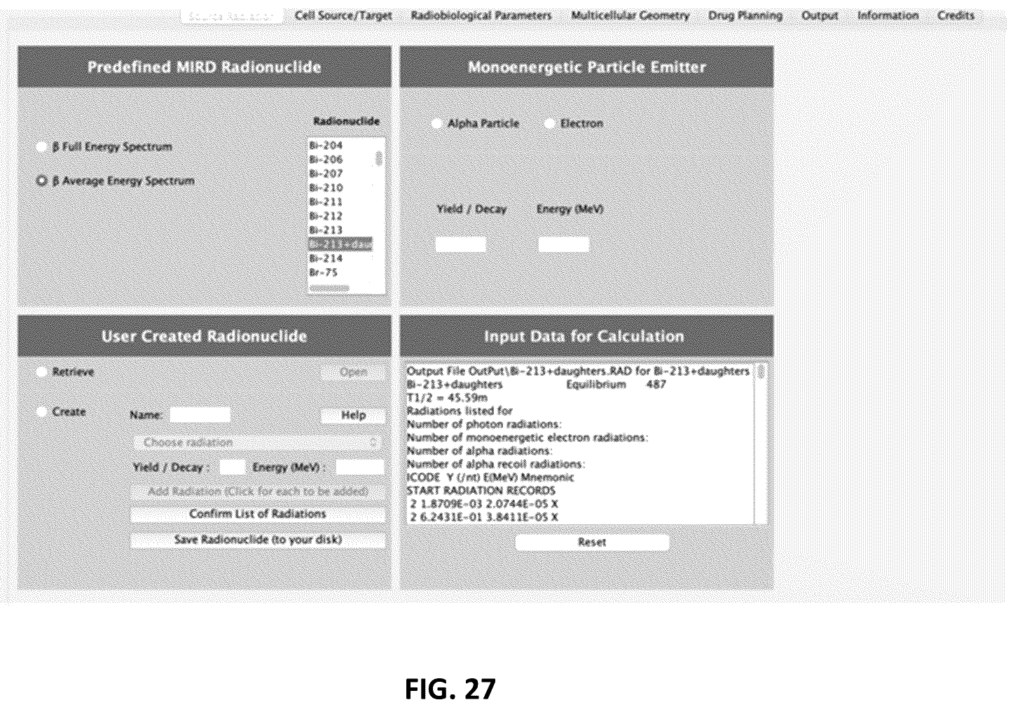

FIG. 27 illustrates an example GUI for an enhanced Source Radiation feature. This feature allows a user to select the radioactivity in the source cells (i.e., cells labeled with radioactivity). Upon making a selection, the radiation data may be shown in a field such as the box entitled Input Data for Calculation. Three choices are available: (1) predefined MIRD radionuclide, (2) monoenergetic particle emitter, and (3) user-defined radionuclide. User defined radionuclides include decay chains for ²¹¹At, ²¹³Bi, ²²³Ra, and ²²⁵Ac.

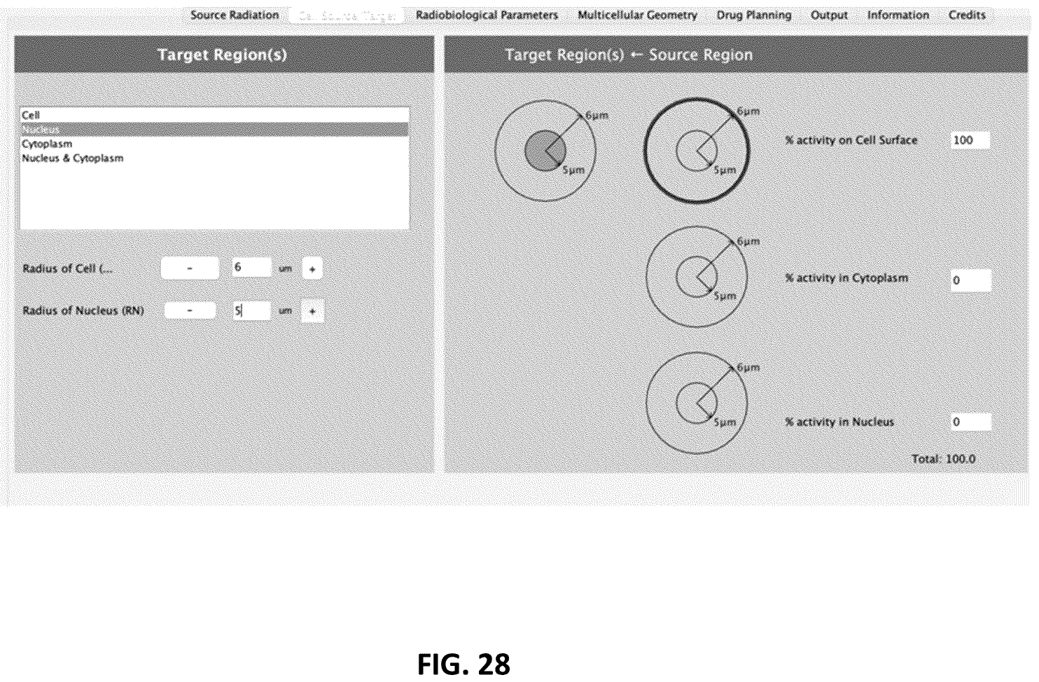

FIG. 28 illustrates an example GUI for an enhanced Cell Source/Target feature. This feature allows a user to select source volume in cell where radioactivity is distributed. cells are modeled as two concentric spheres with radii corresponding to those for the nucleus (RN) and cell (RC), respectively. The cells are modeled as liquid water of unit density. The eligible source regions are cell (C), cell nucleus (N), cytoplasm (Cy), and cell surface (CS). The user may distribute the activity between N, Cy, and CS. The eligible target regions for which the radiation absorbed dose is calculated, and used for bioeffect modeling, are C, N, and Cy. The user may enter the sphere radius in the text box. No limit has been set on the maximum sphere radius; however, extensive testing has only been conducted up to 10 um. Users should be mindful that the effect of photons may be ignored. However, photon contributions to the absorbed dose can become significant for large sphere sizes. Although the algorithms should be adequate for calculating absorbed doses to larger spheres, caution should be exercised when interpreting results for RC>10 μm.

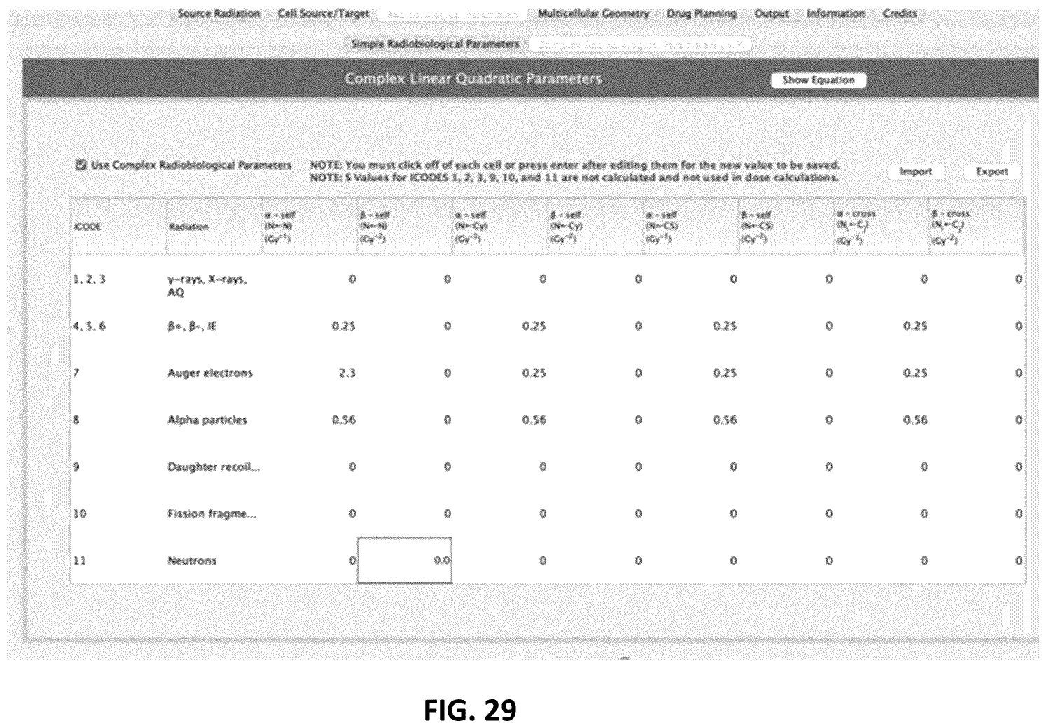

FIG. 29 illustrates an enhanced GUI for a Radiobiological Parameters feature. This feature allows a user to model the surviving fraction (SF) of cells in a specified cell population based on the calculated absorbed doses to the individual cells. Two options are available in V3 for calculating the probability that a given cell survives:
a) the simple linear quadratic (LQ) model described above with respect to FIG. 15
b) an enhanced LQ model. The user may specify LQ parameters not only for self-dose and cross-dose, but also independently for each type of radiation (alpha, beta, Auger, etc.) and for each target region (C, N, Cy). A modified LQ model is again implemented. For example, when the cell nucleus, N, is the target region and the source radiation type is designated by ICODE, the probability that the kth cell survives the insult is given by the equation below:

P_{ICODE}(Nₖ)=e^{α}^{_{ICODE}}^{^{SELF}}^{(N}^{ₖ}^{←N}^{ₖ}^{)D} ^{_{ICODE}}^{^{SELF}}^{(N}^{ₖ}^{←N}^{ₖ}^{)-β}^{_{ICODE}}^{^{SELF}}^{(N}^{ₖ}^{←N}^{ₖ}^{) [D}^{_{ICODE}}^{^{SELF}}^{(N}^{ₖ}^{←N}^{ₖ}^{)]}²×e^{α}^{_{ICODE}}^{^{SELF}}^{(N}^{ₖ}^{←Cy}^{ₖ}^{)D} ^{_{ICODE}}^{^{SELF}}^{(N}^{ₖ}^{←Cy}^{ₖ}^{)-β}^{_{ICODE}}^{^{SELF}}^{(N}^{ₖ}^{←Cy}^{ₖ}^{) [D}^{_{ICODE}}^{^{SELF}}^{(N}^{ₖ}^{←Cy}^{ₖ}^{)]}²×e^{α}^{_{ICODE}}^{^{SELF}}^{(N}^{ₖ}^{←CS}^{ₖ}^{)D} ^{_{ICODE}}^{^{SELF}}^{(N}^{ₖ}^{←CS}^{ₖ}^{)-β}^{_{ICODE}}^{^{SELF}}^{(N}^{ₖ}^{←CS}^{ₖ}^{) [D}^{_{ICODE}}^{^{SELF}}^{(N}^{ₖ}^{←CS}^{ₖ}^{)]}²×e^{α}^{_{ICODE}}^{^{cross}}^{(N}^{ₖ}^{←N}^{ⱼ}^{)D} ^{_{ICODE}}^{^{cross}}^{(N}^{ₖ}^{←N}^{_{numbcell}}^{)-β}^{_{ICODE}}^{^{cross}}^{(N}^{ₖ}^{←N}^{ₖ}^{) [D}^{_{ICODE}}^{^{cross}}^{(N}^{ₖ}^{←N}^{_{numcell}}^{)]}²×e^{α}^{_{ICODE}}^{^{cross}}^{(N}^{ₖ}^{←←Cy}^{ⱼ}^{)D} ^{_{ICODE}}^{^{cross}}^{(N}^{ₖ}^{←Cy}^{_{numbcell}}^{)-β}^{_{ICODE}}^{^{cross}}^{(N}^{ₖ}^{←Cy}^{ⱼ}^{) [D}^{_{ICODE}}^{^{cross}}^{(N}^{ₖ}^{←Cy}^{_{numcell}}^{)]}²×e^{α}^{_{ICODE}}^{^{cross}}^{(N}^{ₖ}^{←←CS}^{ⱼ}^{)D} ^{_{ICODE}}^{^{cross}}^{(N}^{ₖ}^{←CS}^{_{numbcell}}^{)-β}^{_{ICODE}}^{^{cross}}^{(N}^{ₖ}^{←CS}^{ⱼ}^{) [D}^{_{ICODE}}^{^{cross}}^{(N}^{ₖ}^{←CS}^{_{numcell}}^{)]}²

where,

D_{ICODE}^{self}(Nₖ→Nₖ)=f_{N}Á(Cₖ) S_{ICODE}^{self}(Nₖ→Nₖ)

The ICODEs for the different radiation types are as defined in the MIRD Radionuclide Decay Scheme monograph. Here, f_{N} is the fraction of cell activity in the nucleus, Ã(Cₖ) is the time-integrated activity in the source region Nk and S_{ICODE}^{self}(Nₖ→Nₖ) is the self S-value corresponding to the absorbed dose per decay from Nₖ→Nₖ and is given by the equation below:

$${S}_{ICODE}^{self} ( {N}_{k} ← {N}_{k} ) = {\sum }_{irad = 1}^{iradN} \frac{{Δ}_{ICODE , irad} {ϕ}_{ICODE , irad} ( {N}_{k} ← {N}_{k} )}{m ( {N}_{k} )} .$$

Where the sum runs through all iradN radiations of type ICODE, Δ_{ICODE,irad} is the mean energy emitted per nuclear transition of the irad^{th} radiation of type ICODE, ϕ_{ICODE,irad}(Nₖ→Nₖ) is the fraction of energy emitted from the source region Nₖthat is absorbed in the target region Nₖ of the irad^{th} radiation of type ICODE. The terms corresponding to the self-dose from other cell compartments of the same cell (Cy, CS) can be written similarly, as can the terms corresponding to the cross-doses from other cells. Finally, the overall probability of the k^{th} cell surviving, after the effects of all radiation types on the k^{th} cell nucleus Nₖ, is written as:

$$P ( {N}_{k} ) = {\prod }_{ICODE = 1}^{Number of ICODEs} {P}_{ICODE} ( {N}_{k} )$$

Here, the effect of each radiation type is considered independent of the other. The determination of whether a given cell survives (alive) or not (dead) is determined by a Monte Carlo method where the probability of survival, calculated as described above, is compared with a random number (0≤x≤1).

Default values are arbitrary and the user is cautioned to enter values that are relevant to their application. The user is provided with the option of importing a desired set of LQ parameters and also saving a set of custom parameters used in the model.

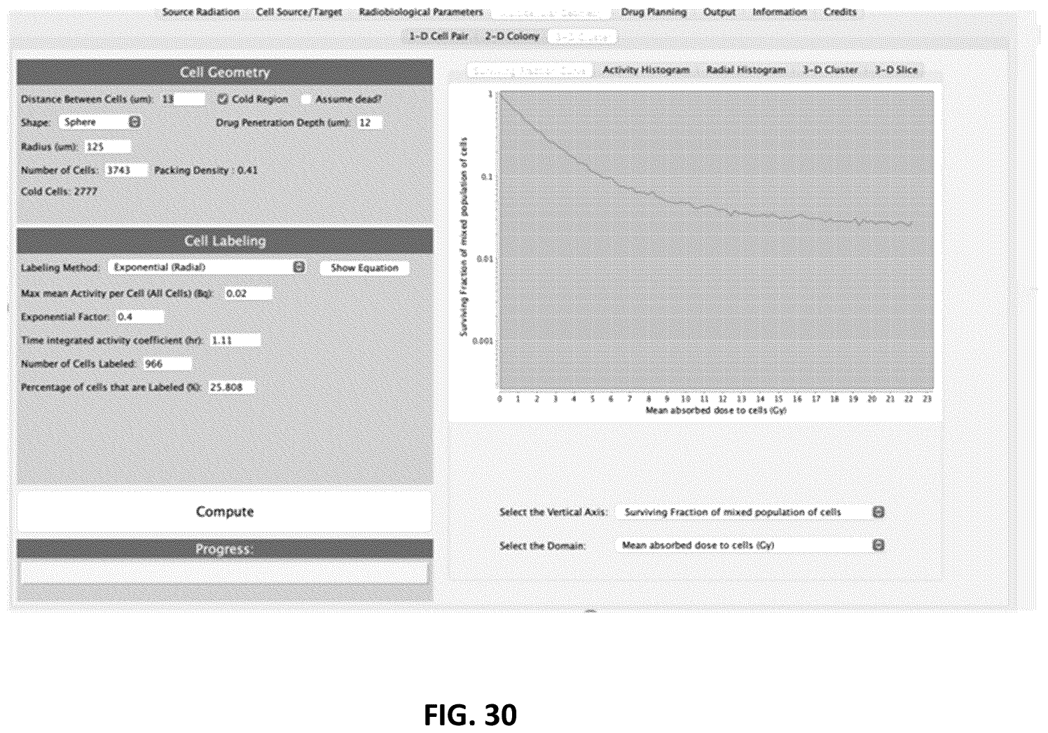

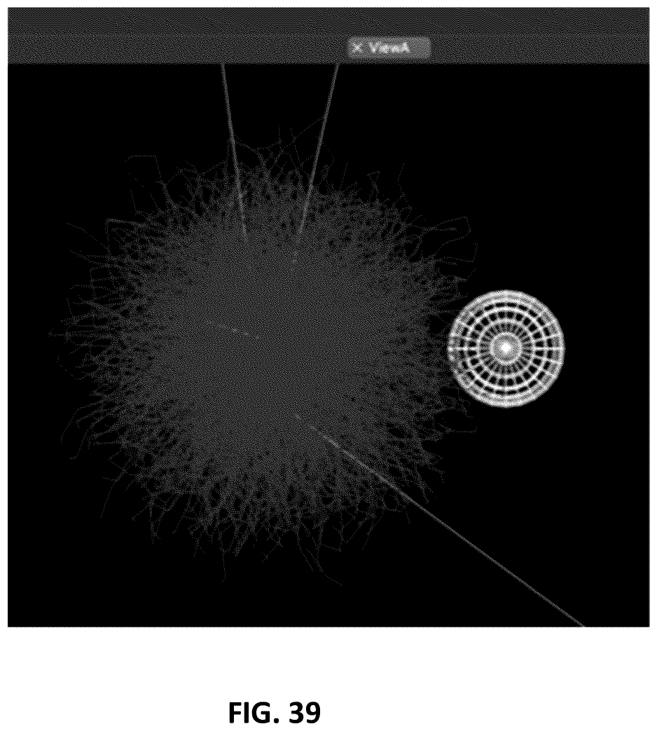

FIG. 30 illustrates an enhanced GUI for a Multicellular Geometry feature. This feature has three basic geometric configurations of spherical cells: 1-dimensional (1-D), 2-dimensional (2-D), and 3-dimensional (3-D). The 1-D option is presented in the 1-D cell Pair tab and it is used to calculate the self- and cross-doses for a pair of cells. The user can set the distance (d) between the centers of two cells. The self-dose and cross-dose S coefficients (also known as S values) are calculated using analytical methods based on range-energy relationships for electrons and alpha particles as described in more detail below, e.g., with respect to FIGS. 39-52.

FIG. 30 also illustrates an enhanced Cell Labeling box sub-feature. This sub-feature includes both built-in and user-provided radial activity distributions. The built radial distributions are linear, exponential, polynomial and four-parameter log-normal distributions. Polynomial distributions up to the 10th degree are possible by setting parameters accordingly. In all the radial activity distributions, r=0 corresponds to the center of the cell cluster. It is important to note that the user-defined activity distribution feature is only available for the spherical cluster geometry. Furthermore, for the ellipsoid cluster geometry, only the standard normal, log-normal and uniform activity distributions are available as cell labeling methods.

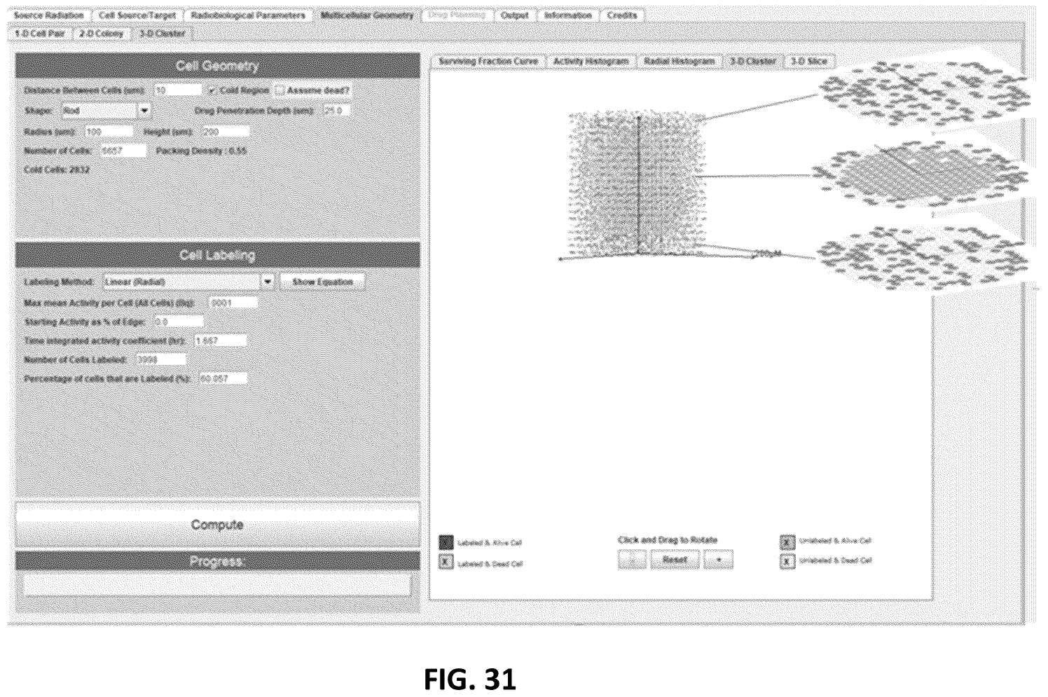

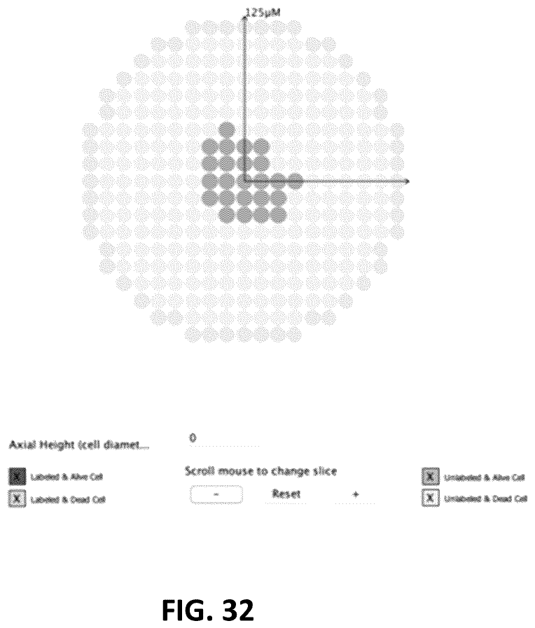

FIG. 31 illustrates an enhanced 3-D Cluster sub-feature. This sub-feature includes tomographic-section visualization. This visualization includes representations of the 3-D cell cluster with color-coded labeled/unlabeled and alive/dead cells, including tomographic sections of each layer of cells (specified in cell diameters) that can be viewed by scrolling the mouse (see FIG. 32).

This sub-feature feature also includes the option of creating a cold region at the center of the cluster and specifying the depth (in um) to which the drug penetrates into the cluster from its outer surface. This situation is common for cluster with radii >50 μm. The cold region at the center of the cluster will contain unlabeled cells. The various activity distributions described in the previous paragraph can be assigned to the outer region of the cluster that has the labeled cells. The complex algebraic algorithms that are used to label cells according to the drug penetration depth are provided for different geometries. FIG. 31 illustrates three tomographic sections of a rod-shaped cluster of cells with a cold region in the interior. Cells that are labeled with a radionuclide may be color coded. And alive cells may be opaque, while dead cells may be translucent cells.

Using the Surviving Fraction Curve of FIG. 31, the Surviving Fraction (SF) of a cell cluster can be plotted as a function of mean activity per cell (Bq), mean absorbed dose to cells (Gy), mean activity per labeled cell (Bq), mean absorbed dose to labeled cells (Gy), mean absorbed dose to unlabeled cells (Gy), mean decays per cell and mean decays per labeled cell. The tumor control probability (TCP) may be displayed on the vertical axis which can be visualized as a function of any of the domains specified above. The TCP is calculated using two different approaches. In the first approach, the TCP is calculated using the Poisson Model expression:

CP(D)=(1−SF(D))ⁿ

where SF(D) is the surviving fraction at a mean absorbed dose D and n is the number of cells in the cluster. The Poisson model of TCP works under the assumption that the number of surviving cells are Poisson distributed with an average nSF(D). The second approach takes the survival probability of each cell into account when calculating the TCP. The TCP is calculated using the following expression:

$$TCP = {\prod }_{i = 1}^{n} ( 1 - {P}_{i} )$$

where Pᵢ is the survival probability of the i^{th} cell.

the output data are written to two boxes in the Output tab. The right-hand box of the Output contains the cellular self- and cross-dose S coefficients for all target←source combinations. The left-hand box of the Output contains most of the information and data used to calculate the absorbed doses and bioeffect. These data are used to create the various plots that are available in “Multicellular Geometry” tab. Output data may be saved as a .txt file. More granular data is provided as well including absorbed doses from each radiation type, radial dose distributions, and other important data used to make the plots.

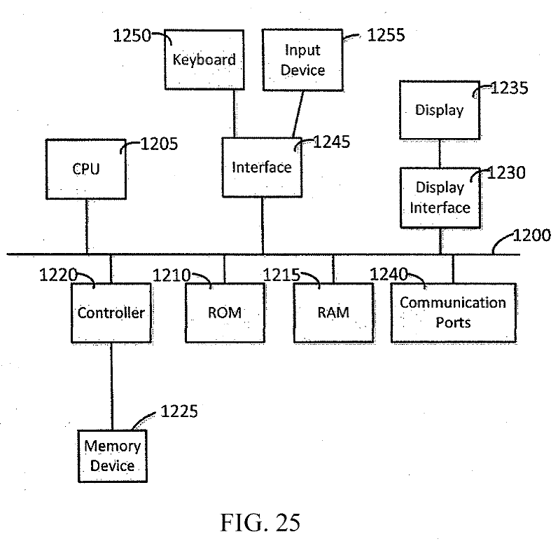

FIG. 25 depicts a block diagram of hardware that may be used to contain or implement an application according to the present invention. A bus 1200 serves as the main information highway interconnecting the other illustrated components of the hardware. CPU 1205 is the central processing unit of the system, performing calculations and logic operations required to execute an application and/or program instructions. CPU 1205, alone or in conjunction with one or more of the other elements disclosed in FIG. 22, is an example of a processing device, computing device or processor as such terms are used within this disclosure. Read only memory (ROM) 1210 and random access memory (RAM) 1215 constitute examples of memory devices.

A controller 1220 interfaces with one or more optional memory devices 1225 to the system bus 1200. These memory devices 1225 may include, for example, an external or internal DVD drive, a CD ROM drive, a hard drive, flash memory, a USB drive or the like. As indicated previously, these various drives and controllers are optional devices.

Program instructions, software or interactive modules for providing the interface and performing any querying or analysis associated with one or more data sets may be stored in the ROM 1210 and/or the RAM 1215. Optionally, the program instructions may be stored on a tangible computer readable medium such as a compact disk, a digital disk, flash memory, a memory card, a USB drive, an optical disc storage medium, such as a Blu-rayTM disc, and/or other recording medium.

An optional display interface 1230 may permit information from the bus 1200 to be displayed on the display 1235 in audio, visual, graphic or alphanumeric format. Communication with external devices, such as a printing device, may occur using various communication ports 1240. A communication port 1240 may be attached to a communications network, such as the Internet or an intranet.

The hardware may also include an interface 1245 which allows for receipt of data from input devices such as a keyboard 1250 or other input device 1255 such as a mouse, a joystick, a touch screen, a remote control, a pointing device, a video input device and/or an audio input device.

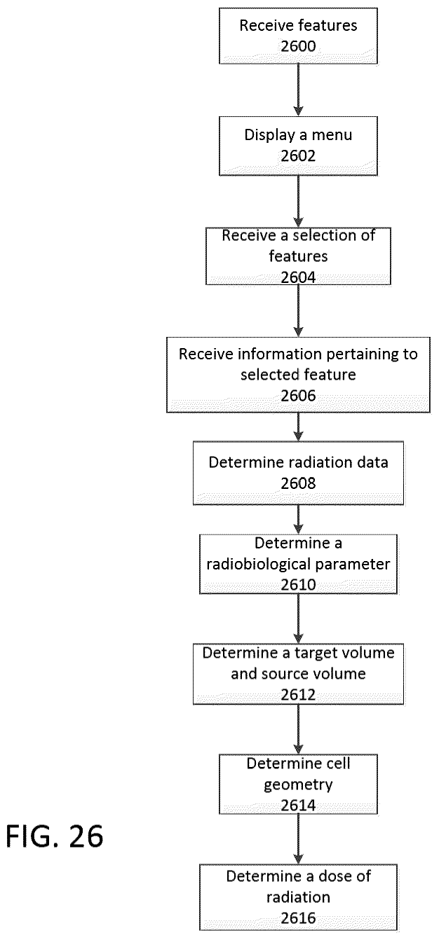

FIG. 26 illustrates a flow chart of an example method of determining a dose of radiation for a patient according to an embodiment. This example is not limited to only determining a dose of radiation, and may include other types of agents to treat infections as well as cancer, such agents include biologic and chemotherapeutic agents. As illustrated by FIG. 26, the method may include receiving by a computing device 2600 one or more features associated with a dose of radiation. In an embodiment, the computing device may display 2602 a menu of one or more of the features associated with the dose of radiation. One or more of the features may correspond to a category of information as previously disclosed above.

In an embodiment, the computing device may receive 2604 a selection of one or more features from the menu of features. For example, the computing device may receive 2604 a selection of one or more features from a user. For one or more selected features, the computing device may receive 2606 information pertaining to the selected feature, determine 2608 radiation data for a radionuclide, determine 2610 a radiobiological parameter, determine 2612 a target volume and source volume, determine 2614 a cell geometry, and determine 2616 a dose of radiation based upon the selected feature. In a further embodiment, a recited feature may include data from a laboratory or patient sample as previously disclosed above.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. All publications mentioned in this document are incorporated by reference. All sizes recited in this document are by way of example only, and the invention is not limited to structures having the specific sizes or dimensions recited below. Nothing in this document is to be construed as an admission that the embodiments described in this document are not entitled to antedate such disclosure by virtue of prior invention. As used herein, the term “comprising” means “including, but not limited to.”

TABLE 1
Organization of an Application and a Graphical User Interface

```
Source Radiation (FIG. 13)
  Predefined MIRD Radionuclide
    β Full Energy Spectrum
    β Average Energy Spectrum
  Monoenergetic Particle Emitter
  User Created Radionuclide
    Add Radiation
    Confirm List of Radiations
  Input Data for Calculation
Cell Source/Target (FIG. 14)
Radiobiological Parameters (FIG. 15)
Multicellular Geometry
  1-D Cell Pair (FIG. 16)
    Distance between cells
  2-D Colony
    2-D Colony (FIG. 17A)
      Cell Geometry
        Number of cells
        Distance between cells
        Shape (Dimensions) (FIG. 17B)
          Circle (specify radius)
          Rectangle (specify length and width)
          Ellipse (specify short and long axis)
      Cell Labeling
        Labeling method
          Uniform
          Lognormal (specify sigma)
          Normal (specify sigma)
        Mean activity per cell (all cells)
        Time-integrated activity coefficient (a)
        Number of cells labeled
        Percentage of cells that are labeled
    Surviving Fraction (FIG. 18)
      SF labeled cells versus mean dose to labeled cells
      SF unlabeled cells versus mean dose to unlabeled cells
      SF all cells versus mean dose to all cells
    Activity Distribution (FIG. 19A, 19B)
  3-D Cluster
    3-D Cluster (FIG. 20A)
      Cell Geometry
        Number of cells
        Distance between cells
        Shape (Dimensions) (FIG. 20B)
          Sphere (specify radius)
          Rod (specify length and width)
          Ellipsoid (specify short and long axis)
      Cell Labeling
        Labeling method
          Uniform
          Lognormal (specify sigma)
          Normal (specify sigma)
        Mean activity per cell (all cells)
        Time-integrated activity coefficient (a)
        Number of cells labeled
        Percentage of cells that are labeled
    Surviving Fraction (FIG. 21)
      SF labeled cells versus mean dose to labeled cells
      SF unlabeled cells versus mean dose to unlabeled cells
      SF all cells versus mean dose to all cells
    Activity Distribution (FIG. 2)
Output (FIG. 23)
Information (FIG. 24)
```

### EXAMPLES

### Predicting Cell Survival Based on Flow Cytometry Gating

Three approaches to modeling the surviving fraction of cells were undertaken. In the first approach, flow-cytometry fluorescence histograms of agent uptake were prepared and the cells were gated relative to control autofluorescence using FlowJo® software (TreeStar). The fractions of agent-negative cells were defined as the proportions of fluorescence spectra that had intensities below the maximum intensities of control samples (i.e., the fraction of fluorescence spectra below maximum autofluorescence). For ²¹⁰Po, 0.1 mM citrate which corresponded to a nontoxic cellular activity of 0.03 mBq/cell was used for autofluorescence. In this simple approach, the gated subpopulations of agent-negative cells were considered as survivors, whereas gated subpopulations of agent-positive cells were considered dead. The surviving fraction was taken as the number of agent-negative divided by the sum of agent-negative and agent-positive cells.

### Predicting Cell Survival Based on Monte Carlo Analysis of Cellular Fluorescence Intensity

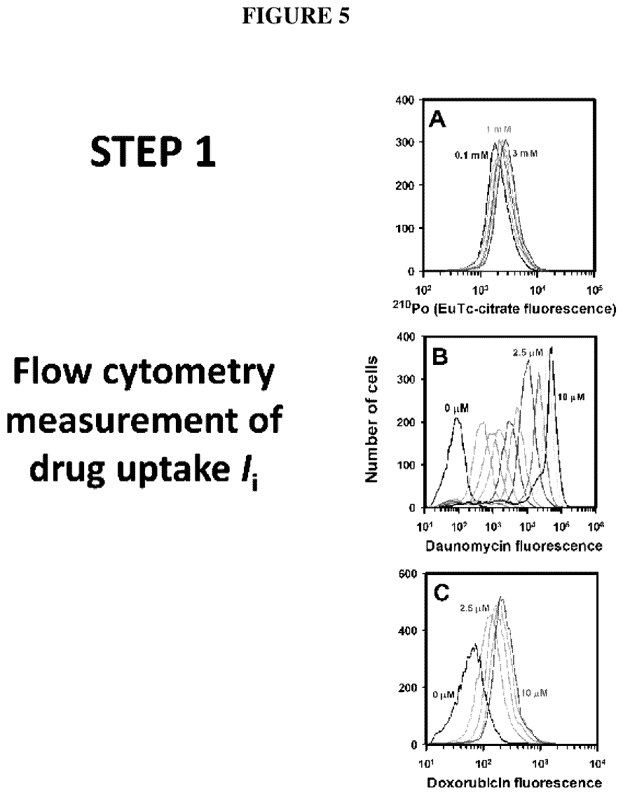

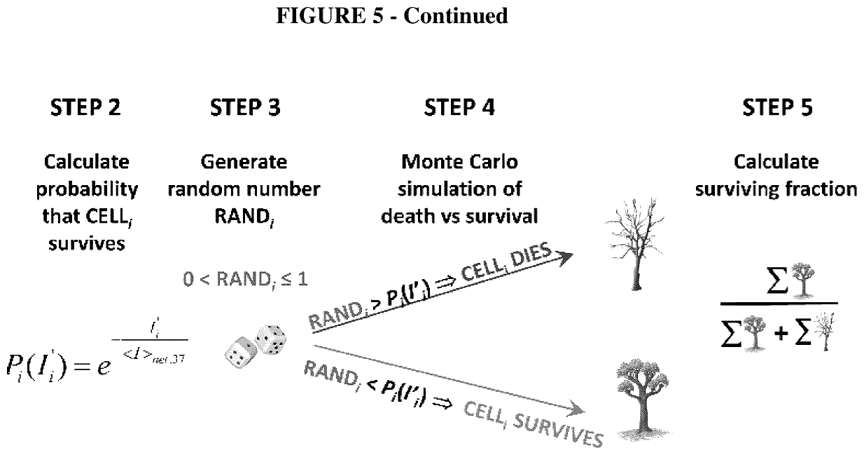

The second approach, depicted in FIG. 5, employs a Monte Carlo simulation that uses experimental individual cell fluorescence intensities Iᵢ for the agent under consideration. The flow cytometry data for citrate, daunomycin, and doxorubicin, consisting of fluorescence intensities emitted by individual cells in a population after incorporation of a fluorescent agent (I₁, I₂, I₃, . . . , Iᵢ), were exported into Microsoft Excel (Redmond, WA) spreadsheets. These raw values were normalized to <I>ₙₑₜ as per the equation below.

$${I}_{i}^{′} = {I}_{i} ( \frac{{〈 I 〉}_{net}}{〈 I 〉} ) ,$$

where

$$〈 I 〉 = \frac{1}{N} {\sum }_{i = 1}^{N} {I}_{i} ,$$

and N is the number of cells analyzed. The cytotoxicity of a therapeutic agent in a given cell is assumed to be exponentially related to the cellular uptake of the agent. Exponential functions are widely used to model the probability of cell death following cytotoxic insults from ionizing radiation and chemicals. Accordingly, the survival probability Pᵢ of the i^{th} cell with normalized fluorescence intensity, I′ᵢ, may be expressed as:

$${P}_{i} ( {I}_{i}^{′} ) = {e}^{- \frac{{I}_{i}^{′}}{{〈 I 〉}_{net , 37}}} .$$

The resulting probability for each cell was compared with a random number generated from a uniform probability distribution by Excel, 0<RANDᵢ<1, and a binary value was assigned to the survival si of the ith cell:

$${s}_{i} = \{ \begin{matrix}0 & dead cell & {RAND}_{i} > {P}_{i} ( {I}_{i}^{′} ) \\ 1 & live cell & {RAND}_{i} \leq {P}_{i} ( {I}_{i}^{′} )\end{matrix}$$

A new random number was generated for each cell. This type of random number approach to determining the fate of each cell was also used in our recent communication (Rajon et al. 2011). Therefore, the surviving fraction of a population of N cells treated with a given concentration of an agent that yields net mean fluorescence intensity per cell, <I>ₙₑₜ, may be expressed as:

$$SF ( {〈 I 〉}_{net} ) = \frac{1}{N} ( {\sum }_{i = 1}^{N} {s}_{i} )$$

Care must be exercised when

$${\sum }_{i = 1}^{N} {s}_{i}$$

is small because the statistical uncertainty of the Monte Carlo calculation of SF is high under such circumstances. This occurs at high agent concentrations that cause low surviving fractions. This is best circumvented by analyzing a larger number of cells. A less preferable alternative is to run additional simulations with new random number sequences and average the results.

### Predicting Cell Survival Based on Mean Cellular Uptake

The third approach uses the same Monte Carlo approach for determining the fate of each cell, however, it is assumed that every cell in the population contains the same amount of drug. That is, each cell is assigned a fixed net mean fluorescence <I>ₙₑₜ which in essence corresponds to case wherein the lognormal shape parameter σ→0. In this instance, the probability of survival of the i^{th} cell is given by

$${P}_{i} ( {〈 I 〉}_{net} ) = {e}^{- \frac{{〈 I 〉}_{net}}{{〈 I 〉}_{net , 37}}}$$

The surviving fraction of the population is obtained using the same Monte Carlo method described above except that above survival probability equation is used.

### MATERIALS AND METHODS

### Cell Line and Monolayer Culture

Chinese hamster V79 lung fibroblasts were used. Two different formulations of minimum essential media (MEMA and MEMB) were used, as known in the literature. All media and supplements were Gibco (Carlsbad, CA), including fetal calf serum (catalog no. 10437, lot no. 539574). For routine maintenance, cells were grown as monolayers in Falcon 25-cm² tissue culture flasks (BD, Franklin Lakes, NJ, catalog no. 353082) at 37° C., 5% CO₂-95% air, and subcultured twice weekly. For experiments, V79 cells (passage 4-11) were transferred into Falcon 225-cm² flasks (BD, catalog no. 353138), and were used upon reaching 80%-90% confluence.

### Suspension Cell Culture

Cells grown in 225-cm² flasks were trypsinized (0.25% trypsin, Gibco, catalog no. 25200-056), and MEMB was added to obtain 2×10⁶ cells/mL. Aliquots of 1 mL were placed in Falcon 17×100 mm polypropylene tubes (BD, catalog no. 352018) and placed on a rocker-roller (Thermo Fisher, Fair Lawn, NJ) for 3 hours at 37° C. with 5% CO₂ and 95% air. After this conditioning period, cells were treated with drug or radiochemical. Cell cultures were exposed to radiochemical and drugs for 0.5 and 2.5 h, respectively.

### Cellular Incorporation of ²¹⁰Po-Citrate, Daunomycin, and Doxorubicin

²¹⁰Po-Citrate. The uptake of ²¹⁰Po-citrate was determined on a cell-by-cell basis by flow cytometric techniques, using ²¹⁰Po-free citrate. Briefly, V79 cells (2×10⁶ cells/mL) were treated with 0-3 mmol/L of citrate and incubated on a rocker-roller as described earlier. Cellular uptake of citrate was tracked using an europium tetracycline (EuTc) conjugate. Samples were washed 2× with 10 mmol/L MOPS buffer (Sigma, St. Louis, catalog no. M3183), after a 30 min exposure to citrate. The cells were resuspended in 1 mL of MOPS buffer containing EuTc (Sigma, catalog nos. 203254 for Eu and T7660 for Tc), transferred into 7 ml polystyrene flow cytometry tubes (BD, catalog no. 352054), and were incubated at room temperature (˜22° C.) in the dark for 30 min. The final concentration of EuTc was 100 μmol/L. EuTc forms a ternary complex with citrate (EuTc-citrate) which is excitable at 488 nm, and its emission can be captured within the wavelengths transmitted by the 610/20 filter. After washing 2× with MOPS buffer, the samples were resuspended in 1 mL of MOPS buffer, passed 5× through a 21-gauge needle, and were analyzed by flow cytometry using an LSR II flow cytometer (BD), equipped with a 488 nm laser. Cellular incorporation of citrate, expressed in terms of the fluorescence intensity per cell or mean fluorescence intensity (MFI) of EuTc-citrate, was used as a surrogate measure cellular uptake of ²¹⁰Po-citrate.

### Daunomycin and Doxorubicin

To determine the cellular uptake of daunomycin and doxorubicin, the cells were treated with 0-10 μmol/L of each drug in MEMB and incubated on a rocker-roller for 2.5 h. The cells were washed 2× with phosphate buffered saline (PBS), resuspended in 1 mL of PBS, passed 5× through a needle, and were immediately subjected to flow cytometric analysis. The 488 nm laser was used to excite intracellular daunomycin and doxorubicin, and the emission spectra were captured within the wavelengths transmitted by the 575/26 and 530/30 filters, respectively. Cellular incorporation of drugs was also expressed as MFI.

### Toxicity of ²¹⁰Po-citrate

²¹⁰PoCl₄ in 2 mol/L HCI was obtained at 370 MBq/mL from Eckert&Ziegler Isotope Products (Valencia, CA, catalog no. 6310). ²¹⁰Po-citrate was prepared as follows. Briefly, PoCl₄ solution was mixed with 1 mol/L sodium citrate in the ratio of 1:7 (final pH 5.8), and was diluted with MEMB to a volume of 4 mL (final pH 6.9). One milliliter of MEMB containing 210Po-citrate was added to the 1 mL of conditioned V79 cultures (2×10⁶ cells/mL), to arrive at a concentration of 0-250 kBq/mL (pH 6.9-7.0). After incubating for 30 min, the cells were washed 2× with MEMB, resuspended in 2 mL of MEMB, and incubated on a rocker-roller for 2.5 h to simulate concomitant drug exposure. The cells were resuspended in a 5 mL of MEMB, passed 5× through a needle, and counted with a Beckman Coulter Model Z2 (Brea, CA). Aliquots (500 μL) of the cell suspension were transferred to vials, mixed with 5 mL Ecolume (MP Biomedical, Solon, OH, catalog no. 882470), and counted with a Beckman Coulter LS6500, and the mean activity per cell was determined (efficiency, 50% as per prior studies). Aliquots of about 5×10⁵ cells were counted in triplicate for ²¹⁰Po activity and the cpm ranged from 10³-10⁵. The triplicate measurements kept statistical variations to a minimum. Each sample was serially diluted and plated in Falcon 60×15 mm tissue culture dishes for colony formation. Cultures were incubated for 7 days, and the colonies were fixed in 95% ethanol, stained with 0.01% Amido Black, washed in tap-water, air-dried, and counted.

### Biologic Clearance of ²¹⁰Po

To determine the biologic clearance of ²¹⁰Po from the cells, 4×10⁶ cells/mL were treated with ²¹⁰Pocitrate as described above. After two washes with MEMB, the cells were resuspended in 5 mL of MEMB, passed 5× through a needle, and Coulter counted. Aliquots of 500 μL of cells were transferred to vials and mixed with Ecolume. The remaining cell suspension was plated into 25-cm² flasks (1.0, 0.5, 0.5, 0.2 and 0.2×10⁶ cells/flask). The cultures were harvested after 24, 48, 72, and 96 h, respectively. Each sample was processed for cell counting and liquid scintillation counting as described. All vials were counted after the last harvest. The ratio of cellular activity at each time point to that immediately after treatment was calculated and plotted.

### Toxicity of Daunomycin and Doxorubicin

After conditioning, the cell cultures were treated with daunomycin (Sigma, catalog no. D8809) or doxorubicin (Sigma, catalog no. 44583) to a final concentration of 0-10 μmol/L in MEMB. The tubes were returned to the rocker-roller for 2.5 h. The cells were then processed for colony formation as described above.

### Analysis of the Flow Cytometry Data for Cocktails of Agents

Samples. Flow cytometry control samples consisted of cells treated with the following agents: 1) untreated, 2) 3 mM citrate, 3) 0.63 UM daunomycin, and 4) 2.50 μM doxorubicin, Daunomycin+citrate test samples were 0.63 μM daunomycin+0.1, 0.2, 0.5, 1.0, 1.5, 2.0, 2.5, or 3.0 mM citrate. Doxorubicin+citrate test samples were 2.50 μM doxorubicin+0.1, 0.2, 0.5, 1.0, 1.5, 2.0, 2.5, or 3.0 mM citrate.

Acquisition. Fluorescence intensity histograms were acquired for each sample using an LSR II flow cytometer (BD). The europium tetracycline-citrate complex, daunomycin, and doxorubicin were excited with a 488 nm laser, and their emission spectra were captured within the wavelengths transmitted by the 610/20, 575/26 and 530/30 filters, respectively.

Analysis. FlowJo software (TreeStar) was used to analyze each sample. Dot plots of forward scatter versus side scatter were created to gate cells from debris. Fluorescence intensities were compensated for overlapping emission spectra.

### RESULTS

### Predicting Cell Survival Based on Flow Cytometry Gating

Cells with fluorescence intensities greater than the maximum autofluorescence were considered as agent-positive, while those with lower intensities were agent-negative. For ²¹⁰Po-citrate and doxorubicin, most cells emerged as agent-negative regardless of agent concentration. This occurred because of the relatively small increase in <I> with increasing extracellular concentration of the agent. The proportion of daunomycin-positive cells consistently increased with increasing drug concentration. Conversely, the proportion of daunomycin-negative cells decreased substantially with increasing drug concentration. When surviving fraction, defined in this instance as fraction of agent-negative cells, was plotted as a function of agent concentration, it was apparent that agent-negativity, based on what might be considered conventional flow-cytometry gating, is not necessarily indicative of the ability of a cell to survive. For ²¹⁰Po and doxorubicin, the fraction of agent-negative cells was found to significantly overestimate cell survival over the entire range of agent concentrations assessed. While there was relatively good agreement between daunomycin-negativity and clonogenic cell survival at low concentrations, the fraction of cells that were apparently drug negative failed to accurately predict survival at higher drug concentrations.

### Predicting Cell Survival Based on Monte Carlo Analysis of Cellular Fluorescence Intensity

To assess the capacity of Monte Carlo simulation of cell death and survival from cellular fluorescence data acquired by flow cytometry, the procedure depicted in FIG. 5 was employed. Only the net mean lethal fluorescence intensity, <I>_{net,37} and the measured fluorescence intensity in each cell of the treated population were needed to theoretically model the surviving fraction following treatment by each drug. Panels A, B and C of FIG. 5 show the lognormal nature of the distribution of measured fluorescence intensities following treatment with graded concentrations each drug. Applying the survival probability equation disclosed above to the two cell populations provides the survival probabilities Pᵢ(I′ᵢ) of each cell in populations treated with 0 or 5 μM daunomycin. By generating a random number RANDᵢ between 0 and 1 (FIG. 5, STEP 3), and comparing it with the survival probability Pᵢ(I′ᵢ), the fate (sᵢ) of each cell was determined by comparing the survival probability to a random number (FIG. 5, STEP 4). The surviving fraction of cells, SI(<I>ₙₑₜ), based on normalized individual fluorescence intensities I′ᵢ, were then calculated using the above equation (FIG. 5, STEP 5). There is a transition from nearly all live cells to nearly all dead cells as the agent concentration is increased.

The process described above for determining the surviving fraction SF(<I>ₙₑₜ) was carried out for each of the cell populations which were treated with 0.1-3 mM citrate. The resulting theoretically modeled surviving fractions SF(<I>ₙₑₜ) are plotted in FIG. 6A for ²¹⁰P-citrate, along with the experimental clonogenic survival data. This process was repeated for daunomycin and doxorubicin to create the theoretical data points in FIGS. 6B and 6C, respectively. The Monte Carlo simulated cell survival is in good agreement with colony forming ability of V79 cells after treatment with the cytotoxic agents.

### Predicting Cell Survival Based on Mean Cellular Uptake

Survival curves based on Monte Carlo analysis wherein each cell in the population contains the same amount of drug are presented as straight, dashed lines in FIG. 6. These provide an important point of comparison with the flow cytometry gating model and the Monte Carlo model that accounts for the lognormal distribution of cellular uptake of the agent.

### Prediction of Multiple Agent Toxicity

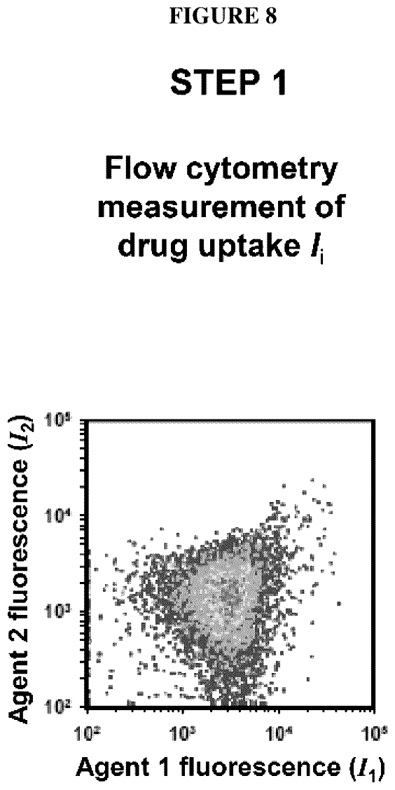

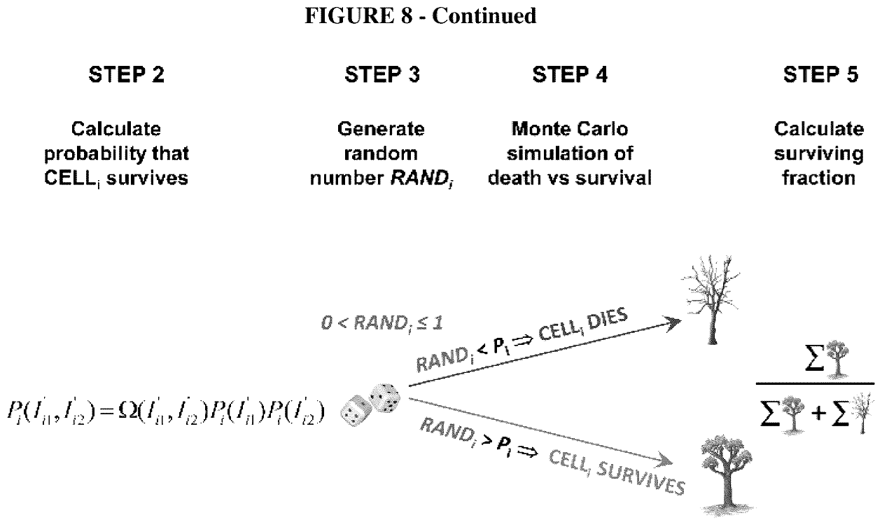

The approach for modeling cell survival using a Monte Carlo simulation is based on individual cell fluorescence intensities Iᵢ for a single agent as described above. When cells were concomitantly treated with multiple agents, the fluorescence intensities of all agents within each cell of each treated population were measured simultaneously using flow cytometry. These data were used to perform a Monte Carlo analysis to simulate the surviving fraction of cells after treatment with all possible combinations of the agents. This process is depicted for a cocktail of agents in FIG. 8.

Flow cytometry of a population of N cells treated with a cocktail of agents provided the fluorescence intensity of each agent in each cell. These data were exported into Microsoft Excel (Redmond, WA) spreadsheets. The raw fluorescence intensities for the j^{th} agent in the i^{th} cell, Iᵢⱼ, were normalized to <Iⱼ>ₙₑₜ for each agent as per the equation below

$${I}_{ij}^{′} = {I}_{ij} ( \frac{< {I}_{j} {>}_{n e t}}{< {I}_{j} >} ) ,$$

where

$$< {I}_{j} > = \frac{1}{N} {\sum }_{i = 1}^{N} {I}_{ij} ,$$

and <Iⱼ>ₙₑₜ denotes the net mean fluorescence intensity per cell following exposure to the j^{th} agent. <Iⱼ>ₙₑₜ is determined by subtracting the mean control autofluorescence, <I>>control, from the mean fluorescence intensity per cell in a treated population as defined by the following equation:

<Iⱼ>ₙₑₜ=<Iⱼ>−<Iⱼ>_{control}

For ²¹⁰Po-citrate, a citrate concentration of 0.1 mM, which was found to correspond to a nonlethal cellular activity of 0.03 mBq/cell, was used for control autofluorescence. The net mean cellular

fluorescence intensity of Agent j that yields 37% survival is denoted <Iⱼ>_{net,37}[20]. To account for natural variations in <Iⱼ>_{net,37} from experiment to experiment, it is necessary to obtain an <I′ⱼ>₃₇ for each experiment from a calibration of an experimentally determined surviving fraction. Assuming that the toxicity of the j^{th} agent in a given cell population is exponentially related to the cellular uptake of the agent, the surviving fraction of such a population based on its net mean fluorescence intensity, <I′ⱼ>, is:

SFⱼ=e^{-<I′}^{ⱼ}^{>/<I′}^{ⱼ}^{>}^{₃₇} (7)

For instance, using the net mean fluorescence intensity of a cell population corresponding to 10% cell survival,

$$< {I}_{j}^{′} {>}_{37} = - \frac{< {I}_{j}^{′} >}{ℓ n ( 0.1 )} .$$

At the single-cell level, the survival probability of the i^{th} cell with normalized fluorescence intensity, I′ᵢⱼ, may be expressed as (FIG. 8, STEP 2):

Pᵢ(I′₁ⱼ)=e^{-I′}^{ᵢⱼ}^{/<I′}^{ⱼ}^{>}^{₃₇} (6A)

Therefore, the survival probabilities for the i^{th} cell when treated with Agent 1 or Agent 2 are given by Pᵢ(I′₁)=e^{-I′}^{ᵢ₁}^{/<I′}^{₁}^{>}^{₃₇} and Pᵢ(I′₂)=e^{-I′}^{ᵢ₂}^{/<I′}^{₂}^{>}^{₃₇}, respectively.

To model cell survival following treatment with a cocktail of two agents, we hypothesize that a cell may die due to Agents 1 and 2 working independently or interactively. The survival probability Pᵢ(I′₁, I′₂) of the i^{th} cell is represented by:

Pᵢ(I′₁, I′₂)=Ωᵢ(I′₁, I′₂) Pᵢ(I′₁)Pᵢ(I′₂)

$$Ω ( {I}_{i 1}^{′} , {I}_{i 2}^{′} ) = \{ \begin{matrix}1 & agents work independently \\ {P}_{i} ( {I}_{i 1}^{′} ) {P}_{i} ( {I}_{i 2}^{′} ) & agents work interactively\end{matrix}$$

where Ωᵢ(I′₁, I′₂) is a term that accounts for the interaction of the two agents.

The probability calculated with the above equations was then compared with a random number, 0<RANDᵢ≤1 (FIG. 8, STEP 3), and a binary value was assigned to the survival si of the i^{th} cell:

$${s}_{i} = \{ \begin{matrix}0 & dead cell & {RAND}_{i} > {P}_{i} ( {I}_{i 1}^{′} , {I}_{i 2}^{′} ) \\ 1 & live cell & {RAND}_{i} \leq {P}_{i} ( {I}_{i 1}^{′} , {I}_{i 2}^{′} )\end{matrix}$$

A new random number was generated for each cell. This approach to determining the fate of each cell (FIG. 8, STEP 4) was also used in our recent communications. Therefore, the surviving fraction of a population of N cells treated with Agent 1+Agent 2 is given by (FIG. 8, STEP 5):

$${SF}_{i} ( {I}_{i 1}^{′} , {I}_{i 2}^{′} ) = \frac{1}{N} ( {\sum }_{i = 1}^{N} {s}_{i} )$$

One embodiment of the invention employs a Monte Carlo approach to simulate the fate of each cell based on its experimentally determined drug uptake and used this information to calculate a surviving fraction for the entire cell population. The resulting surviving fractions were compared to experimentally determined values. Two different methods of predicting cell survival following a toxic insult were considered. The first approach addressed the role of individual agent uptake (i.e., cell fluorescence) in cell survival. The fate of individual cells can be determined based on their incorporation of a given agent, in this case daunomycin. However, it is worth noting that the magnitude of a cell's survival probability, per se, is not conclusive as to whether a cell survives or dies. Hence, there is a need to simulate the fate of each cell within the population using Monte Carlo techniques.

In FIG. 6, experimentally determined cell survival data are compared with theoretical cell survival data that were simulated by Monte Carlo analysis. However, before applying the Monte Carlo approach to account for the experimental lognormal uptake distributions, it is instructive to first see how this model behaves when each cell is assumed to contain the same uptake (i.e., the mean uptake). As expected, when theoretical survival is simulated assuming that each cell in the population contains an agent quantity corresponding to the net mean cellular fluorescence intensity, the resulting survival curve is monoexponential (FIG. 6, dashed lines). In this approach, the fact that each cell in the population is assumed to incorporate the same amount of agent implies σ→0. The data clearly show that, regardless of the agent, the theoretical survival derived from mean cellular fluorescence can recapitulate clonogenic survival only within the first exponential component of cell kill.

In contrast to the poor match between experimental data and the mean approach, Monte Carlo simulation of cell survival in a manner that accounts for the lognormal uptake distribution provides a very good prediction of clonogenic survival following treatment with ²¹⁰Po-citrate, daunomycin, and doxorubicin (FIG. 6). Moreover, this model accurately predicts the transition between the first and second components of cell killing. This transition coincides with that observed when the lognormal shape parameter is plotted as a function of drug concentration. It should be noted that in the case of daunomycin, the Monte Carlo approach accurately predicts cell survival at low drug concentrations within the first log of cell kill but there is some deviation from experimental values at high drug concentrations (FIG. 6B). Nevertheless, the fit is remarkably good compared with the more conventional approach based on mean drug uptake (FIG. 6, dashed lines). Approaches based on mean uptake adequately predict the response in the low-dose realm, but fail to predict the upward trend in the survival curve that arises as a consequence of the lognormal distribution of drug uptake.

### Toxicity of Cocktails

To evaluate cytotoxicity of combined treatment of V79 cells with ²¹⁰Po-citrate and daunomycin, or ²¹⁰Po-citrate and doxorubicin, the surviving fraction of clonogens was plotted as a function of mean cellular uptake of ²¹⁰Po (FIG. 9) and as a function of mean absorbed dose to the cell nucleus. When cells were only treated with ²¹⁰Po-citrate, the survival curve can be described by a 1-component exponential function. When cells were concomitantly treated with a single drug concentration and varying amounts of ²¹⁰Po-citrate, the dose-response curves followed a 2-component exponential function. The data corresponding to the combined treatments were then corrected for drug toxicity, by dividing the surviving fraction for each datum point by the surviving fraction for drug alone (SF=0.10), and replotted in FIG. 9. Both of the corrected cell survival curves emerged below those obtained for ²¹⁰Po-citrate alone. This indicates that daunomycin and doxorubicin enhance the radiotoxicity of α-particles in V79 cells. As illustrated in FIG. 9A, the enhancement of α-particle radiotoxicity by daunomycin was independent of radiation absorbed dose beyond about 0.5 Gy (˜0.09 mBq/cell). On the other hand, the increased kill provided by doxorubicin showed a dose-dependent increase (FIG. 9B).

### Predicting Cell Survival Based on Monte Carlo Analysis of Cellular Fluorescence Intensity

The procedure depicted in FIG. 8 was used to assess the capacity of flow-cytometry assisted Monte Carlo simulation to recapitulate cell death and survival after exposure to ²¹⁰Po-citrate+daunomycin or ²¹⁰Po-citrate+doxorubicin. Following STEPS 2-4 of FIG. 8, yields a plot of live and dead cells for fixed daunomycin concentration and varying concentrations of ²¹⁰Po-citrate. The surviving fraction of a given cell population, based on normalized individual fluorescence intensities, I′ᵢⱼ, was then calculated (FIG. 8, STEP 5). The symbols in FIG. 10 represent the theoretically modeled surviving fractions for ²¹⁰P-citrate+daunomycin and ²¹⁰P-citrate+doxorubicin. The theoretical data are superimposed on curves representing least squares fits to the experimental clonogenic survival data that were shown in FIG. 9. The open symbols were obtained assuming independent action of the agents, while closed symbols represent modeled cell survival when the agents are considered to interact. The Monte Carlo simulation that implemented agent-interaction is in excellent agreement with the experimental data.

These data provide experimental evidence that treatment of Chinese hamster V79 cells with a cocktail of ²¹⁰Po-citrate and a chemotherapy drug (daunomycin or doxorubicin) causes cytotoxicity greater than expected based on the lethality of the agents when used alone (FIG. 9). These data show that when the effect of each chemotherapy drug is corrected for, the corrected survival curves do not coincide with that for the ²¹⁰Po alone treatment. The curves emerge below the ²¹⁰Po survival curve. This is indicative of a chemical enhancement of α-particle radiotoxicity, or perhaps that cells that are not adequately labeled with ²¹⁰Po-citrate (e.g., low end of the lognormal uptake distribution) are killed by the chemotherapy drug. The flow-cytometry assisted Monte Carlo simulation of cell survival can distinguish these possibilities.

In the cocktail embodiment, the flow cytometry-assisted Monte Carlo model has been applied to agent-incorporation data obtained after treating cells with a cocktail of ²¹⁰Po-citrate+daunomycin or ²¹⁰Po-citrate+doxorubicin. The test of the capacity of this approach to predict the cytotoxicity of a combination therapy is presented in FIG. 10 where experimentally determined cell survival curves (solid curves) are compared with theoretical cell survival as simulated by Monte Carlo analysis (symbols). When it is assumed that the agents act independently, the model fails to predict clonogenic cell survival (FIG. 10, open symbols). However, when the agents are assumed to interact, the Monte Carlo simulation predicts cell survival exquisitely at all intracellular activities of ²¹⁰Po (FIG. 10, closed symbols). This suggests that, at the levels of cell kill observed, the chemotherapy agents provide a chemical enhancement of the α-particle radiotoxicity in V79 cells rather than killing cells that are inadequately labeled with ²¹⁰Po-citrate. The latter possibility may become important at very high levels of cell kill (i.e., 5 or 6 logs of kill).

The cocktail approach accurately predicts the experimental toxicity of the ²¹⁰Po-citrate+daunomycin/doxorubicin cocktails based only on knowledge of the initial slope of the dose-response curves for each agent (i.e., <Iⱼ>_{net,37}) and the cellular uptake distribution of the ingredients of the cocktail. This can be extremely helpful in designing more effective cocktails for targeted therapy; these cocktails may consist of a sizeable number of agents.

Although the use of α-emitting radionuclides in radioimmunotherapy has gained considerable interest, the relatively high potency of α-particles limits the amount of activity that can be administered. To benefit from the potency of α-particles and yet maintain low normal tissue toxicity, the use of low doses of cocktails of α-particle based radioimmunotherapeutics and chemotherapy drugs that effectively target all malignant cells is warranted. Yet, only one study has been reported to demonstrate enhancement of the anti-tumor effects of α-particles in a mouse tumor model by paclitaxel. While the exact mechanism of that enhancement is not known, it was suggested to be dependent on the sequence of the administration of the therapeutic agents, and was both angiogenic and apoptotic by nature. Predicting response to radionuclide therapy and chemotherapy drugs on a cell-by-cell basis enables the dissecting of mechanisms involved with drug interaction, and thereby improves the design of more effective cocktails for targeted therapy. Therefore, it is possible that agents previously discarded on the basis of single-agent toxicity may become key ingredients in a cocktail by virtue of their capacity to target a few cells that only incorporate small amounts of the primary drug.

Of particular importance in the above-described embodiments is the fact that the flow-cytometry assisted Monte Carlo simulation of cell survival requires knowledge of only the initial slope of the dose-response curve (i.e., <I>_{net,37}) and the uptake distribution of the radiopharmaceutical or drug. With these two pieces of information, the entire clonogenic survival curve can be recapitulated including both the 1- or 2-component exponential shapes. The approach applies equally well for drugs that are not likely to be characterized by a lognormal uptake distribution such as Hoechst 33342, whose uptake is directly proportional to DNA content.

Special attention should be given to the process of normalizing the fluorescence data obtained by flow cytometry. The measured fluorescence intensities are dependent on flow cytometry hardware (e.g., laser wavelength and intensity), settings (e.g., amplification), etc. Given that the fluorescence intensity is ultimately related to drug uptake in terms of quantities such as mass (g) and/or activity (Bq), there will be a need to implement calibrations for these quantities. The mean activity per cell can be readily measured with high accuracy and precision using standard radiation detection devices. This information, along with the distribution of cellular fluorescence intensities, provides detailed knowledge of the activity in each cell of the population. Furthermore, calibration with drugs with known specific activity (Bq/g) can provide detailed knowledge of the mass of drug in each cell of the population. Accordingly, survival probabilities might be represented by

$${P}_{i} = {e}^{- \frac{{m}_{i}}{< m {>}_{37}}} and {e}^{- \frac{{a}_{i}}{< a {>}_{37}}} ,$$

respectively, where the mass of drug in the cell, mᵢ=ξ I′ᵢ and/or the amount of radioactivity in the cell aᵢ=κ I′ᵢ. The constants ξ and K represent the slopes of plots of <m> and <a versus <I>ₙₑₜ, respectively. These probabilities would be independent of flow cytometry hardware and instrument settings.

Not specifically addressed are the underlying reasons why the experimental and Monte Carlo derived clonogenic survival curves deviate most from those for an average concentration of agent at the higher concentrations. Although not wishing to be bound by any theory, in the realm of chemotherapy, this is often ascribed to resistant subpopulations that may express high levels of the multidrug resistant protein MDR1. One function of this protein is to facilitate the active removal of toxins from the cell, thereby foiling its therapeutic intent. Low cellular uptake by some cells within a population is also especially important for receptor-targeted agents such as radiolabeled antibodies used for radioimmunotherapy. The number of receptors on a given cell can vary widely over a cell population such that sublethal activity may be taken up by a subpopulation. The flow cytometry assisted Monte Carlo embodiment described above can be extremely useful in modeling the consequence of such nonuniformities, thereby reducing the level of experimental effort that is needed to optimize a therapy. Furthermore, a variety of other capabilities can be built into the model to account for other toxic insults to the cell population such as cross-dose received from radiations emitted by neighboring cells, and radiation- or chemically-induced bystander effects.

The use of flow cytometry to predict clonogenic survival using either agent-negative subpopulations of cells or flow cytometry-assisted Monte Carlo simulation has been demonstrated in the present disclosure. Generally, the fraction of apparently agent-negative cells cannot predict cell survival as determined by colony forming ability. However, it has been demonstrated that Monte Carlo simulation using cellular agent incorporation based on individual cell fluorescence intensity of therapeutic agents is a suitable predictor of cell survival. This flow cytometry based approach, which takes explicit account of the lognormal distribution of cellular uptake of the agents, offers a rapid means for determining treatment response on a cell-by-cell basis, and is invaluable in the selection of agents for the design of highly effective therapeutic cocktails that are capable of targeting the diversity in tumor cell populations. Such cocktails can be created not only for treatment of cancer, but also for infectious diseases and other diseases that may be amenable to targeted therapies. Furthermore, the single-cell Monte Carlo embodiment can be used to resolve difficulties encountered when attempting to predict biological response at the multicellular level using macroscopic mean agent doses.

Data are used in this example to model the radiotoxicity of ²¹³Bi bound to the surface of EMT-6 or Line 1 tumor cells grown as spheroids. Briefly, monoclonal antibody 13A to murine CD44 was labeled with ²¹³Bi (²¹³Bi-MAb13A). Only the outer cell layer of the spheroid was labeled such that the activity was localized to a 10 μm layer from the spheroid surface. The dosimetry was performed using Monte Carlo methods using an assumed nuclear radius of 5.35 μm. The average spheroid diameter in their FIG. 6 was 250 μm. Based on their Table 4, we estimated that a cluster of this diameter had 3743 cells. These and other parameters set below were used for both EMT-6 and Line 1 tumor cells as per Kennel et al. (21).

### Methods

From the Source Radiation tab in MIRDcell, the ●β Average Energy Spectrum of ²¹³Bi+daughters is selected (FIG. 27). By selecting ²¹³Bi+daughters, all radiation types emitted by the daughters of ²¹³Bi decay chain are taken into account in the model and the daughters are assumed to be in equilibrium with the parent. The radiation data are displayed in the Input Data for Calculation box.

a. In the Cell Source/Target tab the nucleus is selected as the target region and a single source region is the cell surface. The radius of the nucleus is set to 5 μm. The radius of the cell and the Distance Between Cells (um) in the Multicellular Geometry tab are adjusted until the number of cells in the spherical cluster matches the experimental observations (3473). This requires a cell radius of 6 μm and a distance between equal to 13 μm (FIG. 28).

Since the ²¹³Bi decay chain involves many different radiation types and the LQ parameters vary depending on the type of radiation and the target←source regions, the Complex Radiobiological Parameters are used rather than the Simple Radiobiological Parameters (FIG. 29). Kennel et al. (Radiotoxicity of bismuth-213 bound to membranes of monolayer and spheroid cultures of tumor cells) reported a DO of ˜1.8 Gy using a planar alpha particle source for both cell lines used in their experiment. Therefore, the αparameter for alpha particles in the LQ model is changed to 1/1.8 Gy-1˜0.56 Gy-1. Default values are kept for the other radiation types. It is important to note that the model was also run with zeros for all the parameters of Auger electrons and beta particles and the results were the same as were with default values (i.e., Auger electrons and beta particles play no significant role in the response).

From the Multicellular Geometry tab, 3-D Cluster is selected and the radius of cluster is set to 125 μm. The Distance Between Cells (um) is adjusted until the number of cells match the experimental observations (see “b” above). A Drug Penetration Depth (um) of 12 μm is set and a radial exponential activity distribution is selected from the drop-down menu under Labeling Method; the Exponential Factor was set to 0.4. It important to note that, since the drug penetrates only to a single cell layer (˜12 μm), the selection of the activity distribution has minimal effect on the rest of the cluster. The Time integrated activity coefficient (hr) is set to Tp/ln(2) =1.11 h (where Tp is the physical half-life of the radionuclide). Even though what ultimately matters is the product of the time integrated activity coefficient and the maximum mean activity per cell, which gives you the mean number of decays per cell (after correcting h to s), it is helpful to know the time-integrated activity coefficient for reasonability checks. The Percentage of cells that are Labeled (%) in MIRDcell is set 100%. The Max mean Activity per Cell (All Cells) (Bq) is adjusted until the maximum mean absorbed dose to cells in the MIRDcell Surviving Fraction Curve matched the maximum “Average Dose (Gy)” given in FIG. 6 of Radiotoxicity of bismuth-213 bound to membranes of monolayer and spheroid cultures of tumor cells. When the Compute button is clicked the first time, an error message will pop up indicating that 100% of the cells cannot be labeled as it exceeds the number of cells within the drug penetration depth; it will automatically set the percentage of labeled cells to the maximum number allowed when the error message is accepted. Therefore, a good rule of thumb is to let the program decide the percentage of labeled cells when a drug penetration depth is specified. Alternatively, if there is a specific desired percentage, set the value prior to clicking the Compute button. Once the model is run, the SF as a function of different domains can be visualized under the Surviving Fraction Curve tab. The Max mean Activity per Cell (All Cells) (Bq) that matched the desired absorbed dose was 0.02 Bq (FIG. 30). The surviving fraction variation as a function of mean activity per cell is shown on the right.

The radial activity histograms and tomographic sections (FIG. 31) of the selected cell cluster geometry are displayed in the Radial Histogram and 3-D Slice tabs, respectively. Tomographic sections of each cell layer can be displayed by specifying the value, in terms of cell diameters, in the box labeled Axial Height. Alternatively, the sections can be scrolled through using the wheel of a mouse. FIG. 32 illustrates Tomographic section through the center of the spherical cell cluster illustrating the drug penetration depth, labeled cells (red), unlabeled cells (green), alive cells (opaque), and dead cells (translucent). Only unlabeled cells at the center of the cluster are alive.

The Output tab lists the values of all the parameters used in the model along with the results (not shown). The left panel lists all the output data used for the plots that be viewed under the Multicellular Geometry tab. The right panel lists all the self-S coefficients and the cross-S coefficients as a function of the distance between the center of the source cell and center of the target cell.

### Results and Comparison with Experimental Observations

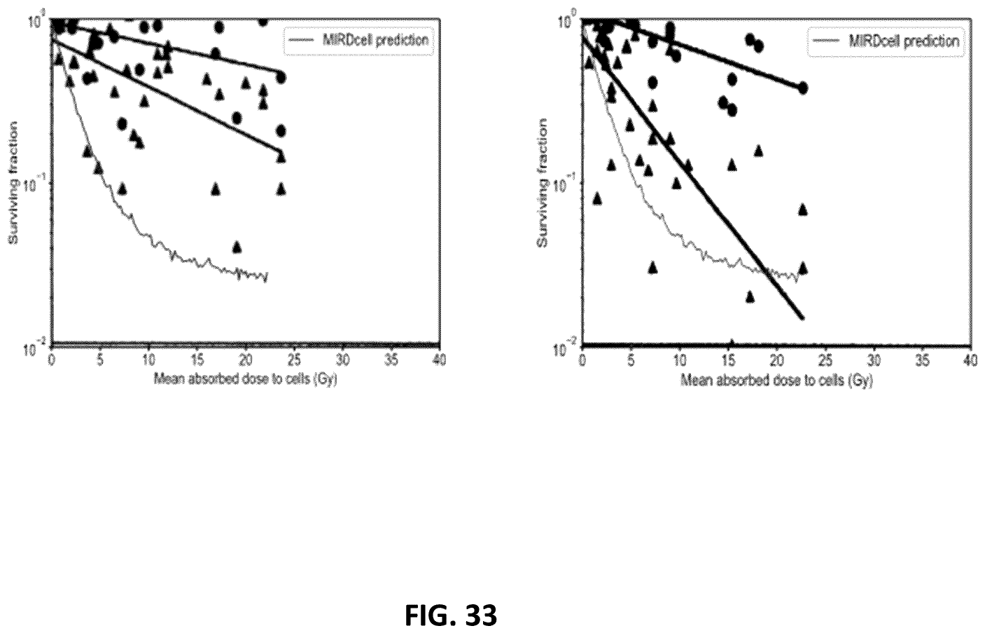

FIG. 33 illustrates a comparison between the experimental observations for the SF as a function of mean absorbed dose for the two cell lines as taken from FIG. 6 of Radiotoxicity of bismuth-213 bound to membranes of monolayer and spheroid cultures of tumor cells. Original plots extracted from Kennel et al. (21) have been overlayed with the MIRDcell predictions (red). A: EMT-6 cells, B: Line 1 cells. Triangles are data obtained for MAb13A and circles are those obtained for MAb14 which is non-binding with tissue. Solid lines are least squares fits to an exponential function provided by Kennel et al. (21). The MIRDcell simulation was run for MAb13A cells. The reader should focus on the triangles which represent the radiolabeled antibody data. The predictions from MIRDcell are given by the red lines. It can be seen from both plots that the data (triangles) are better represented by the MIRDcell prediction than the single component exponential fit used by Kennel et al. Notably, the MIRDcell prediction for the Line 1 cells is superior to that for the EMT-6 cells. Of greatest importance to radiopharmaceutical bioeffect modeling is that MIRDcell predicts the appearance of a tail in the curve as the absorbed dose is increased.

### Predicting Radiotoxicity of ¹¹¹In-EGF Distributed in Spherical Cell Clusters

Falzone et al. studied the effect of 111In-DTPA-human epidermal growth factor (111In-EGF) on the MDA-MB-468 human breast cancer cell line grown as spheroids. The response of MDA-MB-468 was measured after a 1 h or 24 h treatment with the radiopharmaceutical. In this example, the observed biological responses of the MDA-MB-468 spheroids to 111In-EGF is modeled with MIRDcell. This example illustrates how the spatial distribution of the radionuclide within the spheroid, which differs after 1 h and 24 h treatments, affects the growth of the spheroids. Using the information given in (3), the 3-D spheroid diameter is estimated to be ˜450 μm. Falzone et al. reports the cell and nucleus radii of MDA-MB-468 to be 9.46 μm and 6.65 μm respectively. MIRDcell requires integer values for the radii of the cell and cell nucleus, so 9 μm and 7 μm, respectively, are used for modeling. ¹¹¹In-EGF is estimated to have penetrated ˜20 μm into the spheroid after a 1 h treatment with ¹¹¹In-EGF. The biologic response of the spheroids is modeled with MIRDcell in the following way.

METHODS

a. From the Source Radiation tab, the ●β Average Energy Spectrum is selected and from the list, In-111 is selected as the radionuclide. The radiation data are displayed in the Input Data for Calculation box.

b. In the Cell Source/Target tab, the radii of the cell and nucleus are set to 9 μm and 7 μm, respectively. The activity is distributed among the cell surface, the cytoplasm and the nucleus according to the data given in FIG. 3D in Falzone for a 1 h treatment. The relevant subcellular activity distribution given in FIG. 3D after a 24 h treatment should be used when modeling the corresponding biologic response.

Complex Radiobiological Parameters are set in the Radiobiological Parameters tab. In this example, the complex radiobiological parameters, which depend on the radiation type and the source-target regions, are used instead of the simple radiobiological parameters. In the modeling conducted by Falzone, the LQ parameters (α, β) they obtained for MDA-MB-468 cells after ¹³⁷Cs gamma irradiation were used for electrons. Also, in one of the authors' simulations, they have used a relative biological effectiveness (RBE) equal to 4 for the absorbed dose deposited by ¹¹¹In in the nucleus. Accordingly, for this MIRDcell example, the LQ parameter a is increased by a factor of 4 for Auger electrons for the N<-N target←source region, compared to other target←source regions. It is important to note that, since ¹¹¹In produces some internal conversion electrons (IE) in the decay process, one must pay close attention to the LQ parameters used in the β+, β−, IE row. In this example, we have used the α values given in Falzone for ¹³⁷Cs gamma irradiation for the β+, β−, IE row. The α-particle LQ parameter values are left as default values because these will not affect this simulation.

d. In the Multicellular Geometry <3-D Cluster tab, the spheroid radius is set to 225 μm and the distance between cells is adjusted until a packing density of 0.17 is achieved. This matched the packing density of 0.17 that was used by Falzone in the Random Close-Packed (RCP) algorithm of their Monte Carlo model (3). This packing density is achieved when the distance between the cells is set to 26 μm. A drug penetration depth of 20 μm is set in accordance with the results given in (3). The product of the Max mean Activity per Cell (All Cells) (Bq) and the Time integrated activity coefficient (hr) should match the mean number of decays per cell when the latter is corrected to seconds. It is advised to start with 100% as the Percentage of cells that are Labeled (%) and let the program calculate the maximum number of labeled cells that can be accommodated within the drug penetration depth given other dimensional restrictions. This can be done by clicking the Compute button once. Alternatively, if there is a specific desired percentage, set the value prior to clicking the Compute button. As explained in the previous example, from the Output tab, the results as well as all the parameter values can be saved.

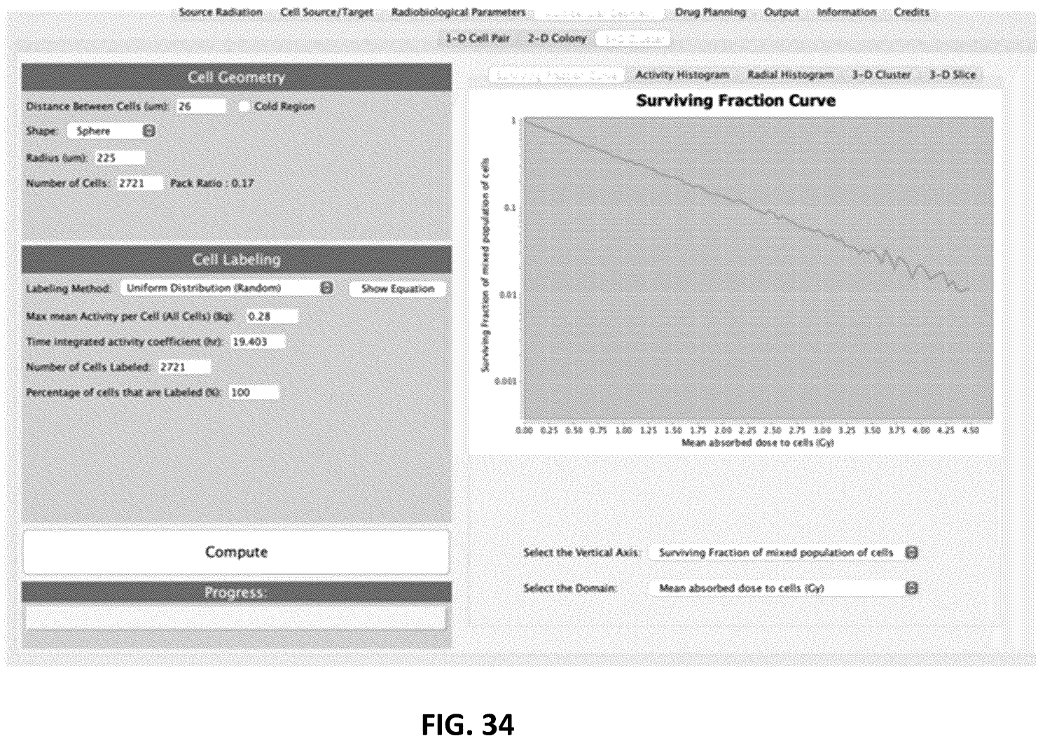

e. Similar modeling with MIRDcell can be done for the 24 h treatment with 111 In-EGF. The activity distribution in this case is: 62% (cell surface), 22% (cytoplasm), 16% (nucleus). Other relevant parameter values are shown in FIG. 34. Note that, a Drug Penetration Depth (um) is not specified here (i.e., the Cold Region box is not checked) because the ¹¹¹In-EGF is fully internalized within the spheroid.

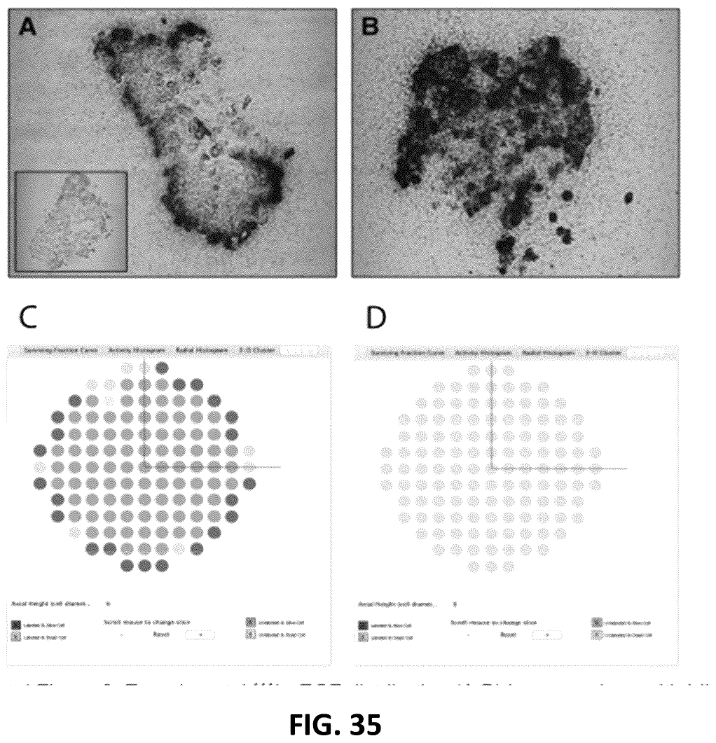

The data obtained from Falzone et al. showed that after the spheroids were treated with ¹¹¹In-EGF for 1 h, the ¹¹¹In accumulated mainly on the periphery of the spheroid (Supplemental FIG. 35A). In contrast, after a 24-h treatment, it was fully internalized (Supplemental FIG. 35B). These distributions were recapitulated by MIRDcell in FIGS. 35C and 35D which illustrate tomographic sections of MDA-MB-468 spheroids, indicating alive and dead cells, after a 1 h and 24 h treatment, respectively.

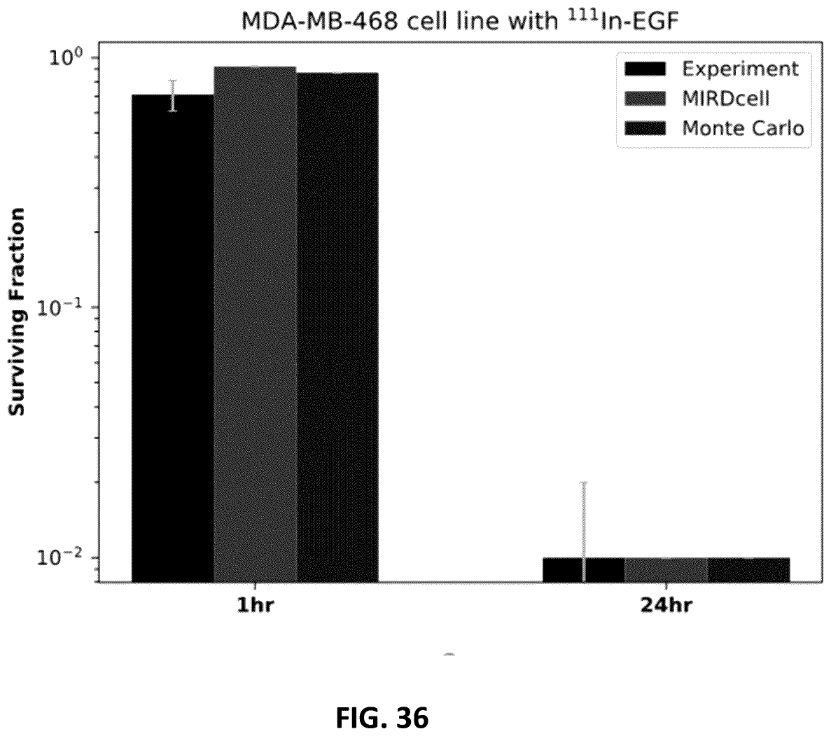

FIG. 36 illustrates a comparison of MIRDcell results for the surviving fraction with experimental and Monte Carlo results reported in Falzone. The Monte Carlo method used an RBE=4 for the absorbed dose from the ¹¹¹In within the nucleus. The MIRDcell predictions are in good agreement with experimental results.

Because of the relatively short range of the Auger electrons emitted by ¹¹¹In, the total absorbed dose to a labeled cell comes mainly from the self-dose while the cross-dose contribution is insignificant (see Output tab). Therefore, the difference in the surviving fraction following 1 h and 24 h treatments is a consequence of the increased percentage of labeled cells in the latter, which in turn increases the total absorbed dose received by the cells in the interior of the cluster.

It is important to note that, when modeling the effects of Auger electron emitters with MIRDcell, the subcellular activity distribution should be considered for multiple reasons. MIRDcell can account for the dependence of the radiotoxicity of Auger emitters based on the source region in which the decays take place.

MIRDcell V3 provides the option to import experimentally measured radial activity distributions for spherical clusters. This permits prediction of the biologic response based on the imported distribution rather than the built-in standard distributions (Uniform, Normal, Linear, Exponential, etc.). This example illustrates the utility of the user-imported activity distribution feature along with the impact of the activity distribution within the cell cluster on the surviving fraction.

Create a csv file containing the radial activity distribution data. The file must have 2 columns separated by a comma. Column 1 is the radial position and Column 2 is the activity per cell (Supplemental FIG. 8). The first radial position of the data should be 0 and the last radial position should match the cluster radius. The data does not have to be absolute activity per cell; relative activity per cell will suffice. With that said, fluorescence intensity data are suitable when fluorescence is used as a surrogate for activity.

From the Source Radiation tab, select the desired radionuclide. In this example, select an electron from the Monoenergetic Particle Emitter section. Set the yield to be 1 per decay and the energy to be 1 keV. By selecting an electron with such a small energy, it is confined to the cell where it is originated, hence minimizing the cross dose to other cells.

From the Cell Source/Target tab, use the default values for the radii of the cell (5 μm) and nucleus (3 μm). Set nucleus as the “Target region” and assign 100% activity to the nucleus.

Use default values for the LQ parameters in the Radiobiological Parameters <Simple Radiobiological Parameters tab.

Click the Multicellular Geometry tab. Then select 3-D Cluster. Select Sphere as the shape and set Distance Between Cells (um) to 10 μm. Set Cluster Radius (um) to 100 μm.

Under Cell Labeling <Labeling Method, choose Import CSV (r, relative A/cell) (Radial) from the drop-down menu. From the window that pops up, navigate to the csv file that contains the data for the activity distribution as a function of radial position and select it. This will import the data to MIRDcell.

Set Max mean Activity per Cell (All Cells) (Bq) and Time Integrated activity coefficient (hr) to 0.01 Bq and 100 h, respectively.

Click the Compute button. This will model the biologic response of the cell cluster to the imported activity distribution of Table 2

TABLE 2

0, 1
2, 1.221402758
4, 1.491824698
6, 1.8221188
8, 2.225540928
10, 2.718281828
12, 3.320116923
14, 4.055199967
16, 4.953032424
18, 6.049647464
20, 7.389056099
22, 9.025013499
24, 11.02317638
26, 13.46373804
28, 16.44464677
30, 20.08553692
32, 24.5325302
34, 29.96410005
36, 36.59823444
38, 44.70118449
40, 54.59815003
42, 66.68633104
44, 81.45086866
46, 99.48431564
48, 121.5104175
50, 148.4131591
52, 181.2722419
54, 221.4064162
56, 270.4264074
58, 330.2995599
60, 403.4287935
62, 492.7490411
64, 601.8450379
66, 735.0951892
68, 897.8472917
70, 1096.633158
72, 1339.430764
74, 1635.98443
76, 1998.195895
78, 2440.601978
80, 2980.957987
82, 3640.950307
84, 4447.066748
86, 5431.659591
88, 6634.244006
90, 8103.083928
92, 9897.129059
94, 12088.38073
96, 14764.78157
98, 18033.74493
100, 22026.46579

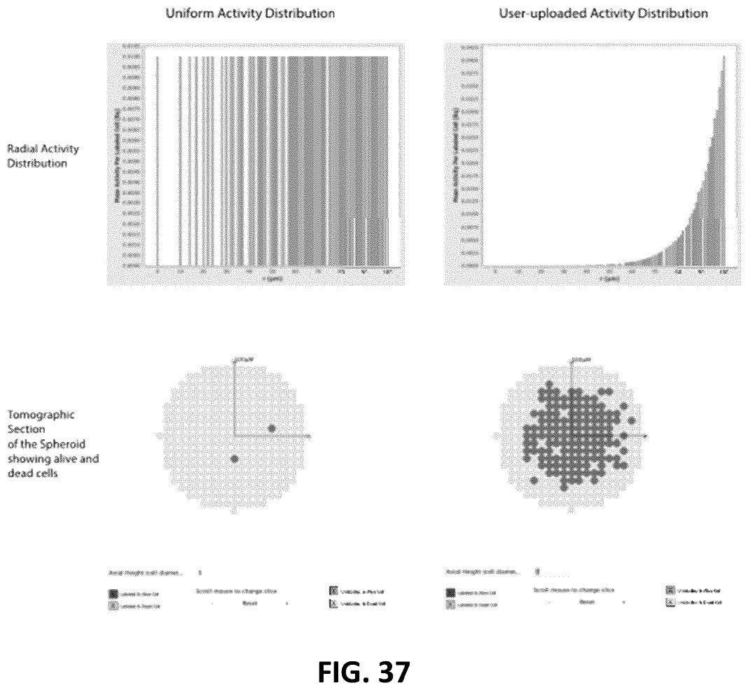

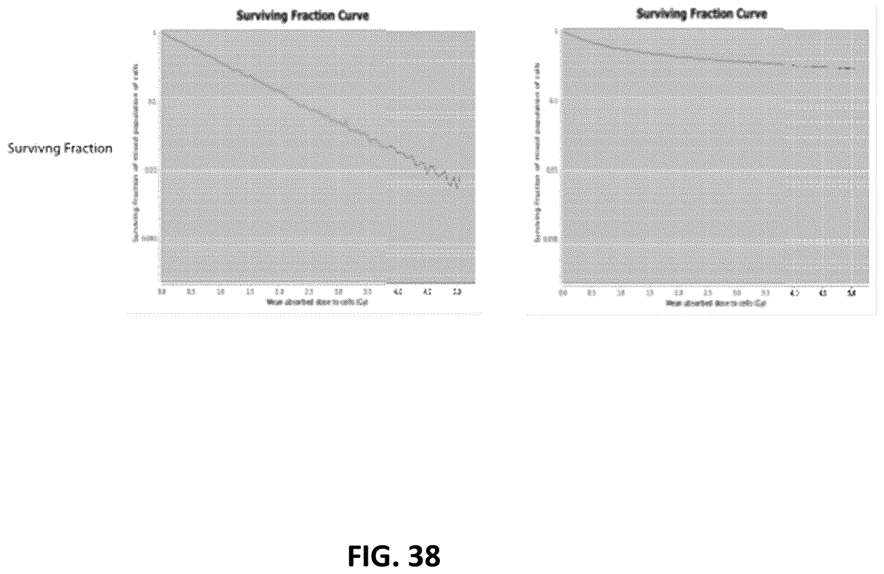

If the same total activity is distributed in a different way within the cluster, the response of the cluster will be different. This can be illustrated in the following way using the same example. Without changing anything else, under Labeling Method, select Uniform Distribution (Random) from the drop-down menu. This will change the activity distribution to a uniform distribution instead of the imported distribution. Since everything else in the model is unchanged, the total activity within the cluster remains the same. A comparison of the surviving fraction curves for the two activity distributions is shown in FIGS. 37 and 38. Row 1 illustrates the radial variation of the activity of the two distributions (from Radial Histogram tab). Row 2 shows the tomographic sections that correspond to the center of each spheroid (cluster), illustrating the distributions of alive and dead cells for the two activity distributions (from 3-D Slice tab). Row 3 shows the surviving fraction curves, as a function of absorbed dose, for the two activity distributions (from Surviving Fraction Curve tab). The distribution of the activity within the cluster has a major effect on the survival curve.

### Comparison of MIRDcell and Monte Carlo Simulation

As described by Goddu et al. (Cellular dosimetry: absorbed fractions for monoenergetic electron and alpha particle sources and S-values for radionuclides uniformly distributed in different cell compartments), the energy deposition of electrons and alpha particles is calculated analytically within MIRDcell using the continuously slowing down approximation (CSDA). The fraction of the i^{th} particle radiation with initial energy, Ei, emitted from the source region, r_{S}, that is absorbed in the target region, r_{T}, is called the absorbed fraction ϕᵢ (r_{T}←r_{S}):

$$S ( {r}_{T} ← {r}_{S} ) = {\sum }_{i = 1}^{𝒩} \frac{{Δ}_{i} {ϕ}_{i} ( {r}_{T} ← {r}_{S} )}{m ( {r}_{T} )}$$

where m(r_{T}) is the mass of r_{T} and N is the number of radiations. The equilibrium dose constant of the i^{th} radiation, Δᵢ=YᵢEᵢ, where Yᵢ is the radiation yield.

In MIRDcell V3.10 and earlier versions, the cells are assumed to be composed of liquid water and the absorbed fraction for electrons is calculated using Cole's experimental range-energy relation for X and, after solving for E, its derivative

$$\frac{d E}{d X} {\vert}_{X} .$$

A modification to Cole's expression was used for electrons with ranges below 0.02 μm. This approach, described in detail in the MIRD Cellular S values monograph along with comparisons of electron S coefficients calculated with the OREC Monte Carlo code, was used for electrons at the inception of the algorithms used for MIRDcell because it is valid for electrons with energies ranging from 20 eV to very high energies, and therefore able to accommodate the wide range of electron energies emitted by radionuclides in the form of Auger electrons, internal conversion electrons, and beta particles. In the case of alpha particles, MIRDcell interpolates the ICRU tables for range and stopping power in liquid water to obtain X and

$$\frac{d E}{d X} {\vert}_{X} .$$

### 50 keV Monoenergetic Electrons

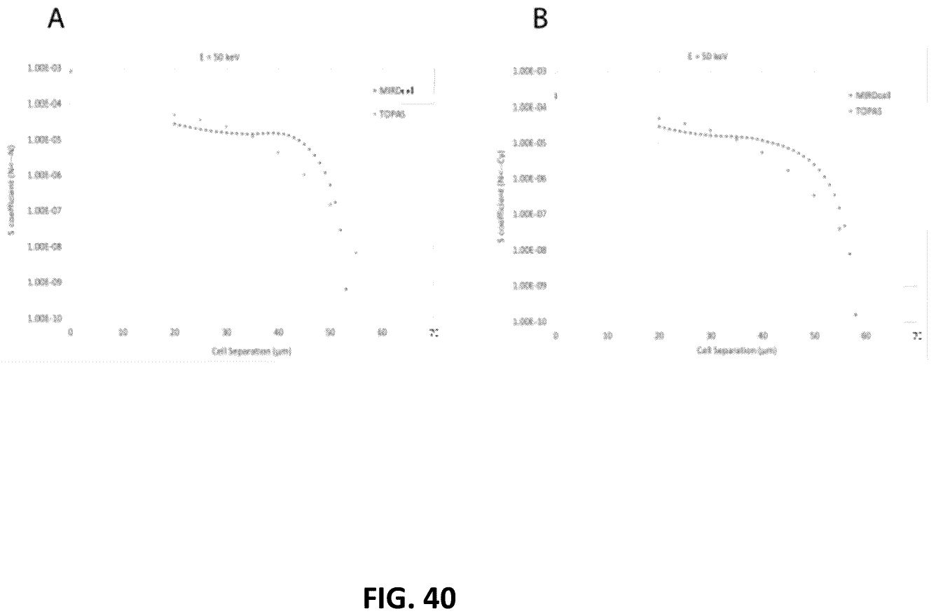

S coefficients for cellular self- and cross-dose were calculated with MIRDcell V3.10 and compared with those obtained via a Monte Carlo (MC) simulation performed using TOPAS nBio, a Geant4-based dosimetry tool. The simulations were carried out with two cells, each composed of liquid water with 10-um cellular radius (RC) and a 5-μm nucleus (RN) radius. The center-to-center distances between the two cells were varied from 20 μm to 60 μm at 1-μm intervals and the S(N←N) were calculated. An identical situation was simulated with TOPAS-nBio with 8,000,000 source particles distributed uniformly within the nucleus of the source cell. The number of histories used for each source particle was one. In both TOPAS-nBio and MIRDcell, the cell and its nucleus were modeled with two concentric spheres (see FIG. 39). The absorbed dose to the target nucleus was scored and the S coefficients were calculated for each cell separation distance. The “production cuts” for electrons was set at 0.01 μm to ensure that each deposition of energy was assigned to the correct subcellular compartment. In TOPAS-nBio (and in Geant4), production cuts control whether a particle is born or not. Therefore, when the primaries no longer have enough energy to produce secondaries which travel at least 0.01 μm, no more secondaries are produced and the primaries are tracked down to zero energy using continuous energy loss. With these settings, a typical TOPAS-nBio run for a given cell separation, in the Rutgers Amarel super-computing cluster, took about 1.5 hours. FIG. 40 illustrates the S coefficients (S(N<-N) and S(N<-Cy)) obtained from MIRDcell and TOPAS-nBio simulations for 50 keV electrons. Notable differences can be seen that depend on cell separation distance.

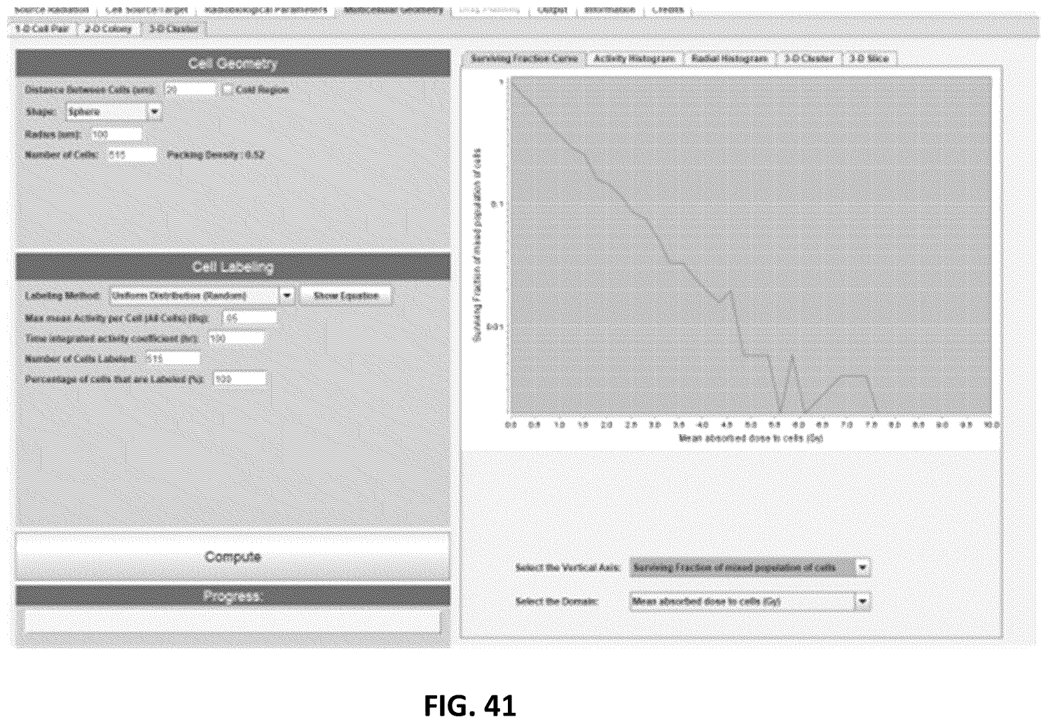

While differences in S values are important, the primary purpose of MIRDcell is to predict the surviving fraction and tumor control probability. Accordingly, the effect of these differences on cell survival are analyzed in more detail below. In MIRDcell, the surviving fraction in a 100-μm radius spherical cluster of cells was estimated using the default values and the same cell and nucleus dimensions and subcellular activity distributions used above were selected. Additionally, the activity distribution within the cluster was set to be uniform. The parameters used and the resulting surviving fraction curve is shown in FIG. 41.

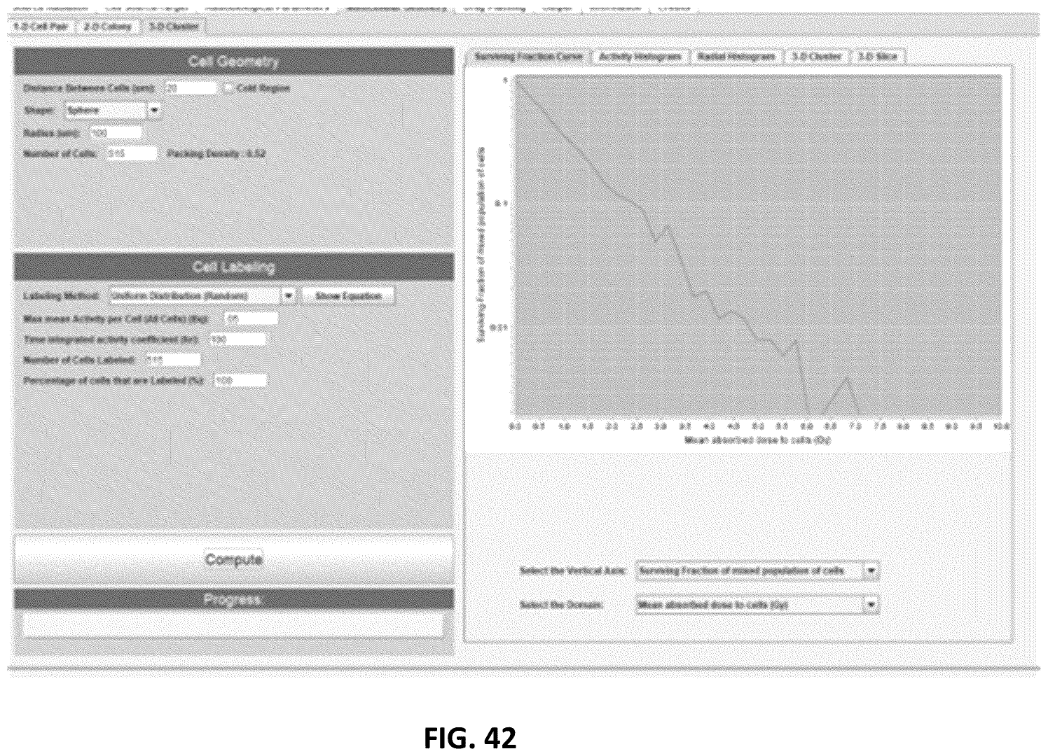

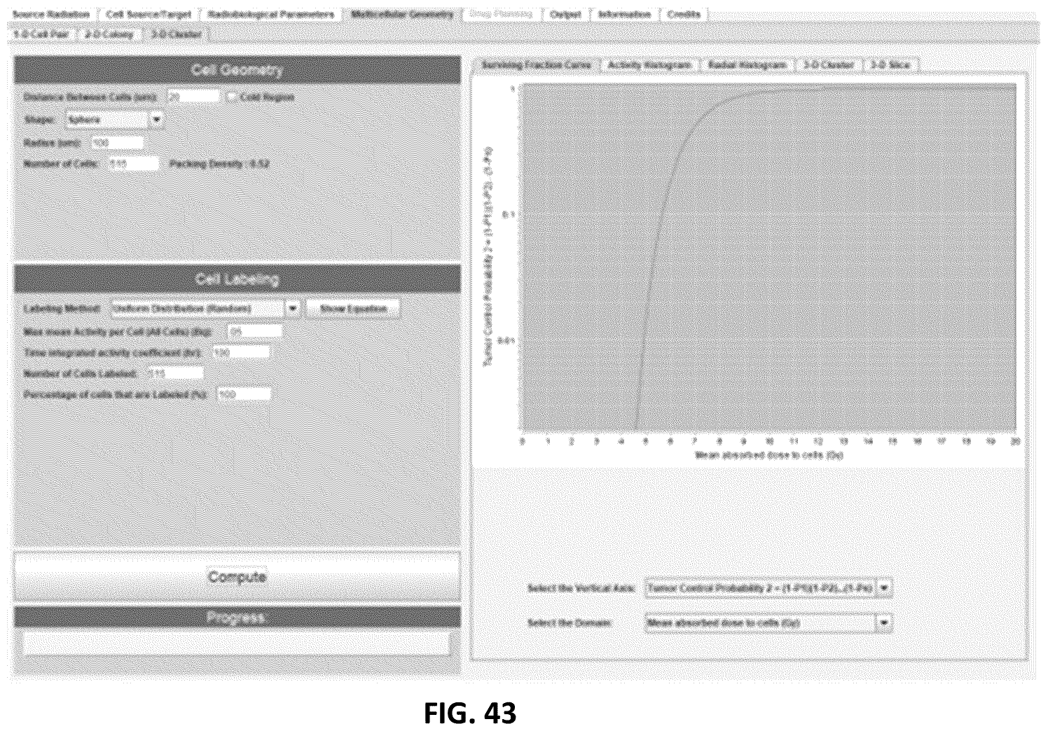

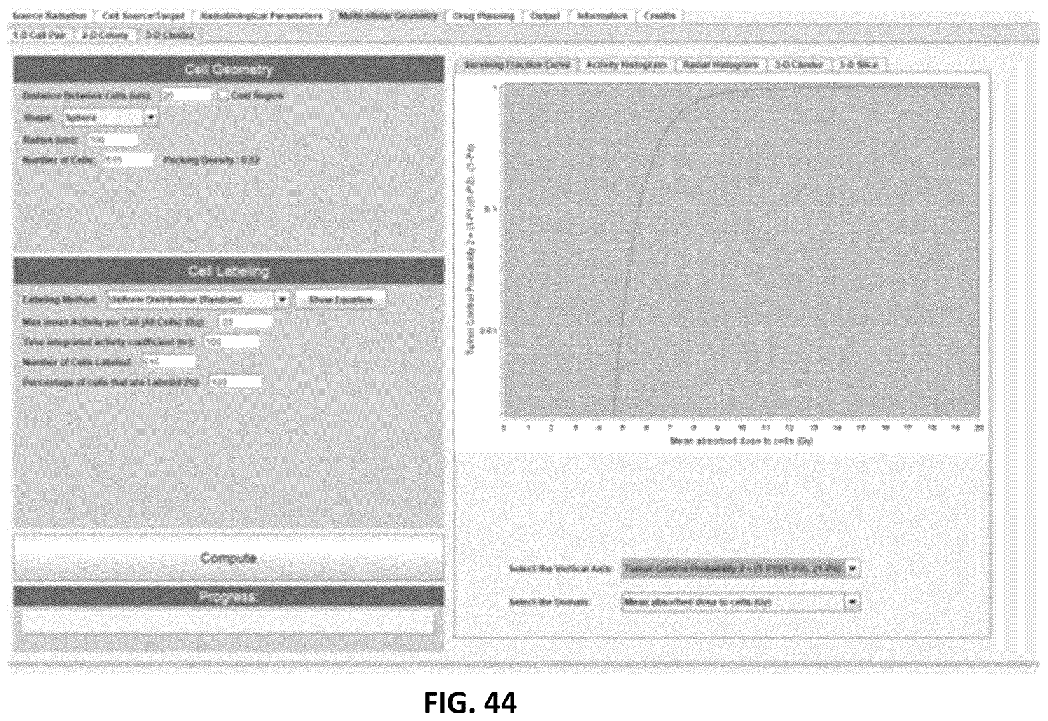

The S coefficients calculated from TOPAS-nBio were then hard-coded to MIRDcell, replacing the ones that are calculated in a typical MIRDcell simulation, to estimate the surviving fraction under otherwise identical conditions as before. The results are shown in FIG. 42. The corresponding Tumor Control Probability (TCP) plots are given in FIG. 43 and FIG. 44 for MIRDcell-generated and TOPAS-nBio-generated S-coefficients, respectively. It can be seen that, for a uniform activity distribution within the spherical cluster, both the surviving fraction and the TCP plots from MIRDcell-generated S-coefficients are similar to those obtained from TOPAS-nBio-generated S-coefficients.

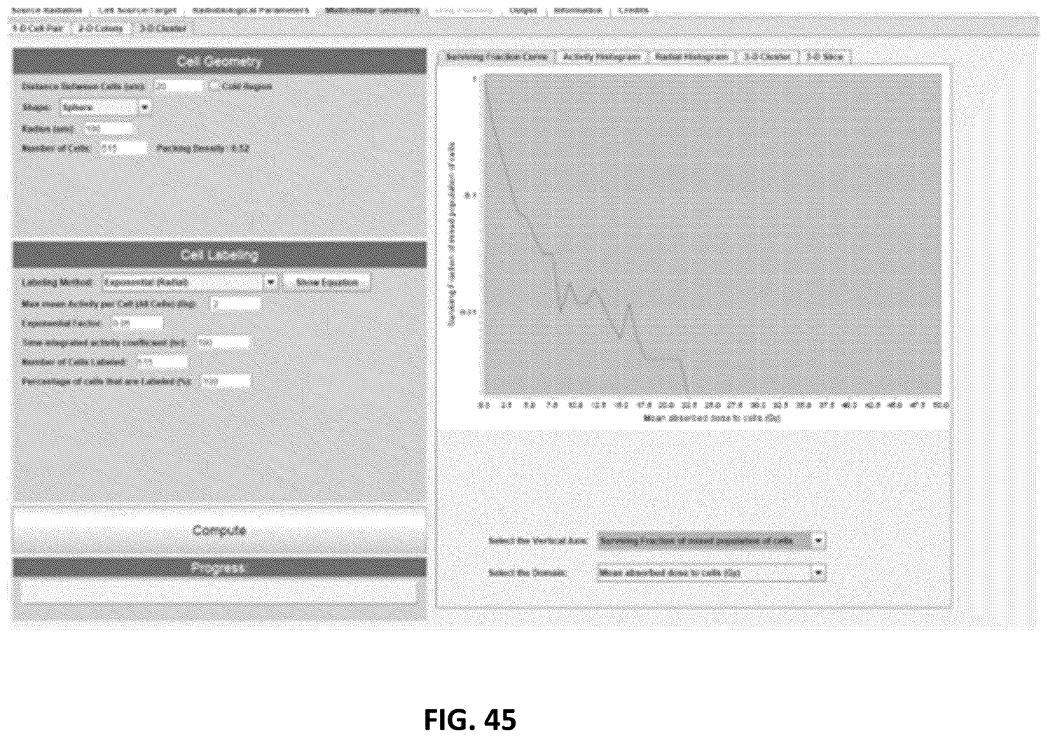

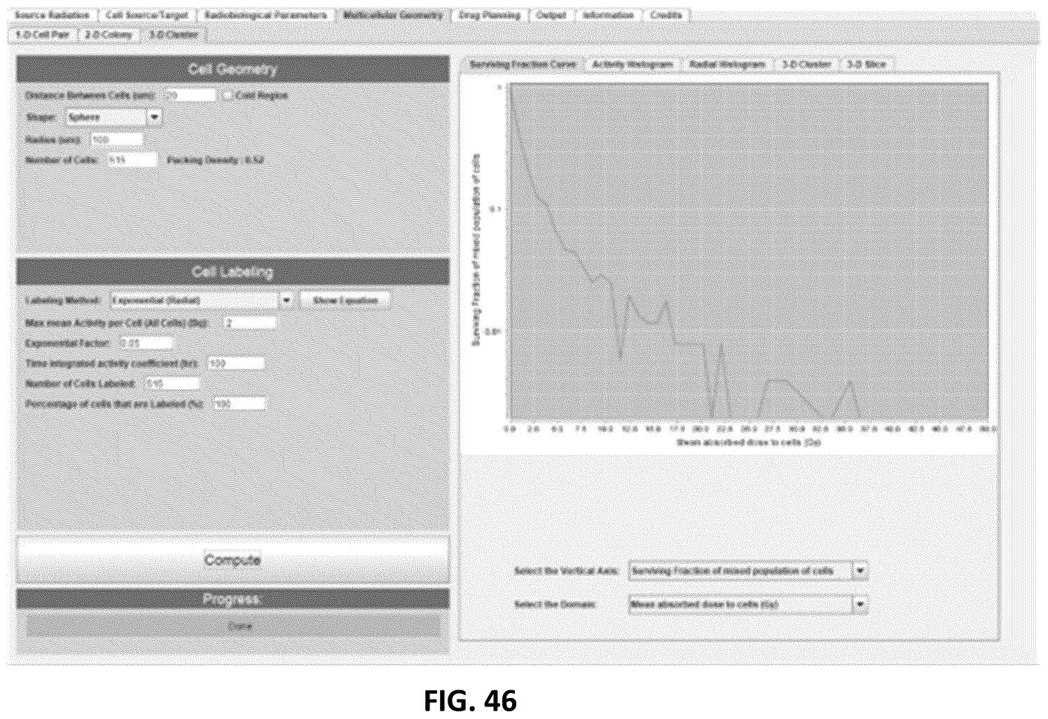

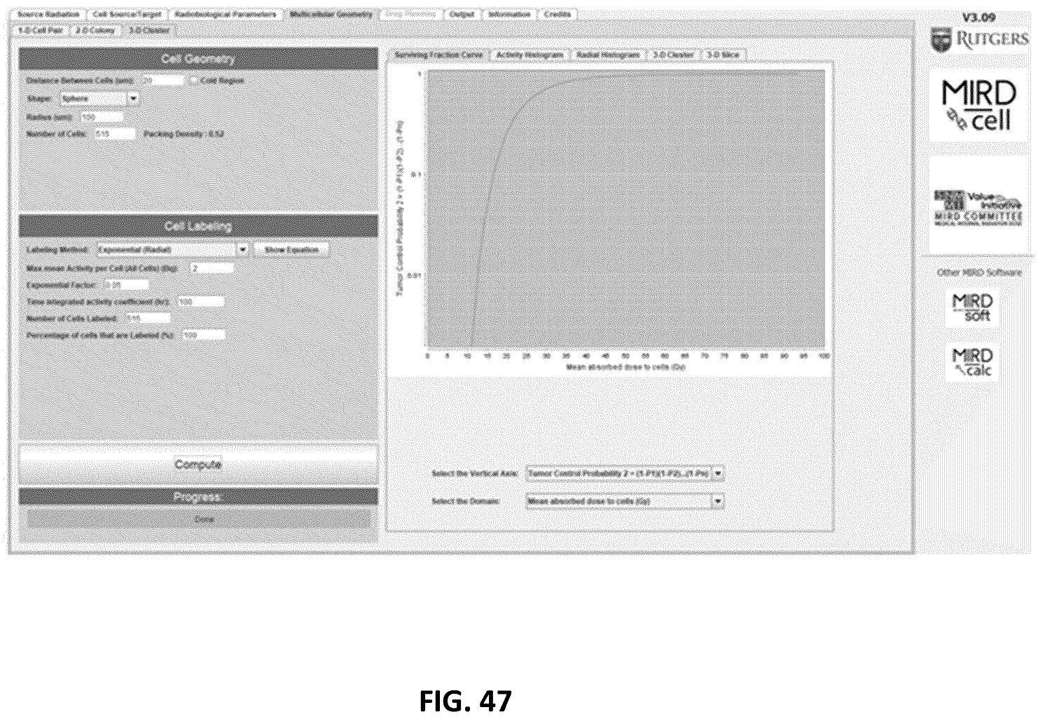

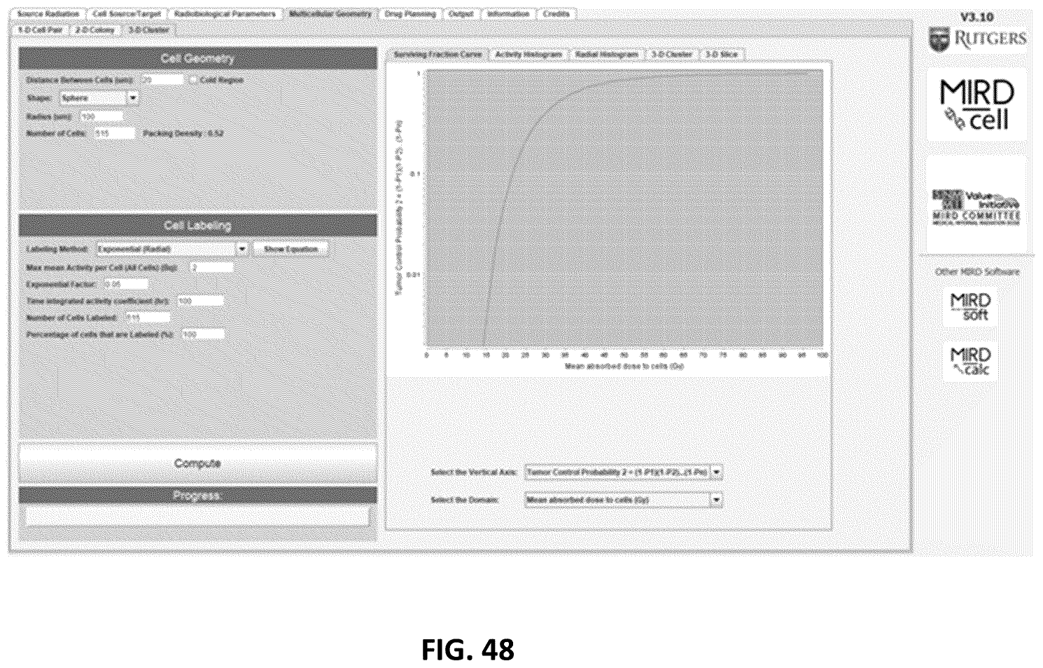

The above exercise was repeated with a radial exponential activity distribution within the spherical cluster, where most of the activity was concentrated in the outer periphery of the cluster. The resulting surviving fraction plots from MIRDcell-generated S-coefficients and TOPAS-nBio-generated S-coefficients are shown in FIGS. 45 and 46, respectively. These two survival curves are somewhat different. The SF plot from MIRDcell-generated S-coefficients overestimates the cell kill when compared to the TOPAS-nBio-generated SF plot. The corresponding TCP plots are modestly different from one another (FIGS. 47 and 48). These results can be explained by the Cole expression used in MIRDcell, which shows marked peaking of electron dE/dR at the end of its track (FIG. 40A). When the activity is largely distributed in the outer periphery of the spherical cluster, a significant fraction of electron tracks will end within the cluster towards its center. Therefore, relative to TOPAS-nBio, MIRDcell will enhance the absorbed dose deposited in cells near the center of the cluster. Nevertheless, MIRDcell does a remarkably good job of predicting effect using analytical approaches that only require short computation times. In contrast, the Monte Carlo simulations that are required for multicellular dosimetry require long-computation times which make them presently unsuitable for calculating the multitude of S values needed for the very flexible multicellular modeling scenarios provided by MIRDcell.

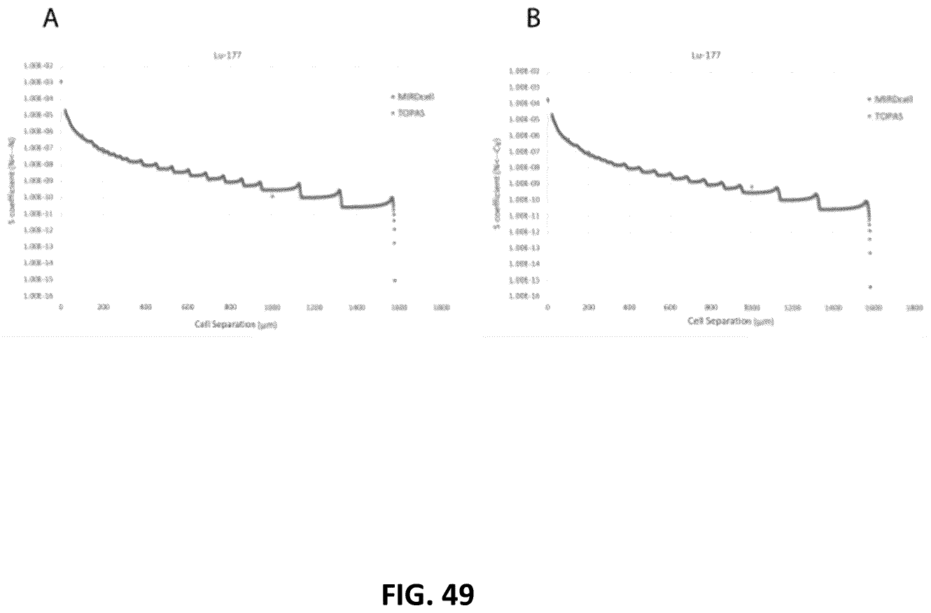

Similar analyses for the S coefficients were done for ¹⁷⁷Lu using both MIRDcell and TOPAS-nBio. In MIRDcell, the full β energy spectrum was used to calculate the S coefficients. In TOPAS-nBio, a production cut of 0.01 μm was applied for all particles during the simulation. The cellular and nucleus dimensions were kept the same as that in the monoenergetic electron-analysis discussed above. A comparison of the S coefficients calculated with MIRDcell and TOPAS-nBio is shown in FIG. 49. For the TOPAS-nBio simulation, only a selected set of cell separation distances (MIRDcell S coefficients were calculated at each um) were used due to limitations related to excessive computation times.

The peaks visible in the S coefficients calculated by MIRDcell (also mentioned by Marcatili et al. (Realistic multi-cellular dosimetry for ¹⁷⁷Lu-labelled antibodies: model and application)) are due to two effects. First, MIRDcell uses the Cole's experimental range-energy relation (discussed in the previous section) in calculating the absorbed fraction of the electron energy by the target, which tends to increase towards the track end of an electron (see MIRDcell-calculated S coefficients in FIG. 40). Second, the full β energy spectrum of ¹⁷⁷Lu used in MIRDcell is not a continuous spectrum, instead, it is a discrete spectrum that is logarithmically binned at different electron energies. Therefore, each peak observed for MIRDcell in FIG. 49 can be attributed to a track end for a particular electron with a given initial energy. Note that the separation distance between the peaks for each electron increases as the cell separation distance is increased. This is due to the logarithmic binning of the discrete beta spectrum used to approximate the continuous beta spectrum. Smoothing of these peaks can be achieved by increasing the number of energy bins with a consequent increase in computing time. In the limit, a smooth curve could be attained and run times would approach those required for TOPAS-nBio.

Noting the differences between MIRDcell and the TOPAS-nBio results for electrons, we have explored the use of analytical range-energy relationships for electrons other than Cole's. Emfietzoglou et.al (10) have introduced an analytical expression, based on a fit to MC-calculated penetration depth in water data (FIG. 3b in Ref. (10)) that is valid for electrons with energies <10 keV. It is given by:

R=a(b+cT)^{d}

where R is the range in nm and T is the electron energy in keV. The fit parameters a,b,c and d, are: a=7.511 × 10^{-6,} b=800.1, c=4702, d=1.783

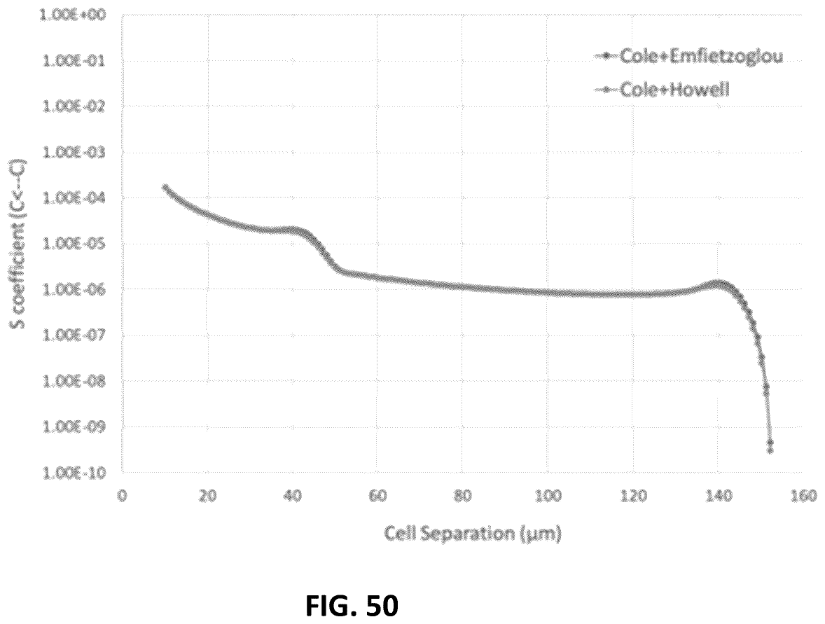

Using MIRDcell, a comparison of S coefficients (S(C←C)) was made between the MIRDcell electron algorithm (Cole's range-energy relation down to residual range of 0.02 μm and the Howell expression (6, 11) for ranges below that) and an algorithm which uses Cole's relation down to an electron energy of 10 keV and the Emfietzoglou expression given above for lower energies. In the model, R_{C}=5 μm and R_{N}=3 μm were used for both source and target cells, and the source was assigned an electron spectrum with two energies: 50 keV and 100 keV, each with a yield of one. A comparison of the calculated S coefficients using the two algorithms is shown in FIG. 50.

It can be seen from FIG. 50 that the two expressions show not only similar behavior but also produce comparable results for the S coefficients. The two “bumps” in the curves at around 43 μm and 140 μm correspond to the CSDA ranges of the 50 keV and the 100 keV electrons respectively. Note that the Cole+Emfietzoglou S coefficients are larger (up to 20%) than the Cole+Howell S coefficients around the 42 μm position.

### 5 MeV Monoenergetic Alpha Particles

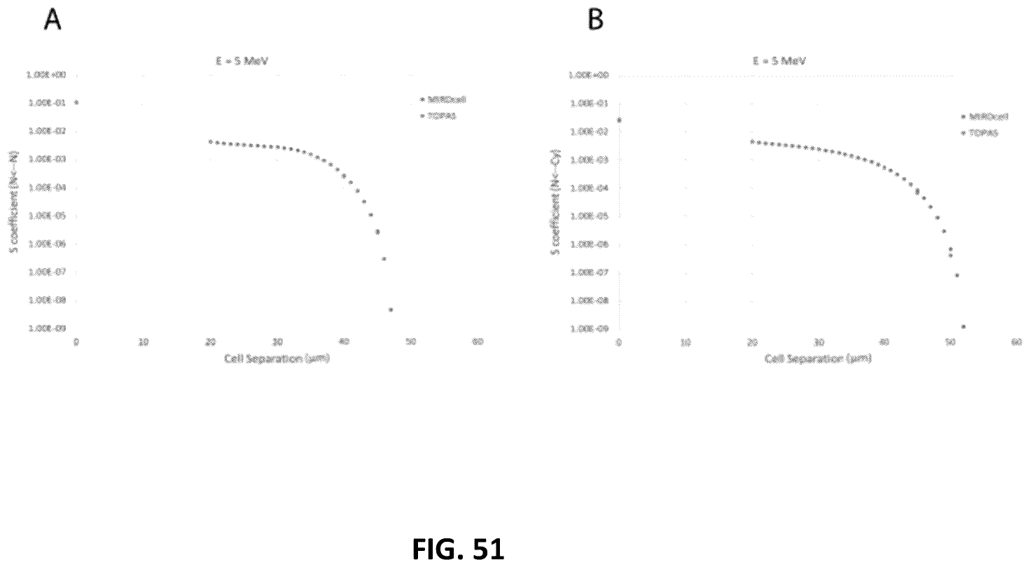

The S coefficients, S(N←N) and S(N←Cy), were calculated for 5 MeV alpha particles using MIRDcell and TOPAS-nBio. The cellular and nucleus dimensions used were R_{C}=10 μm and R_{N}=5 μm. In TOPAS-nBio, a production cut of 0.01 μm was applied for both alpha particles and electrons during the simulation. A comparison of the MIRDcell-calculated and MC-calculated S coefficients is shown in FIG. 51. The self S-coefficients are plotted at the cell separation distance of 0 μm. There is excellent agreement between MIRDcell and TOPAS-nBio.

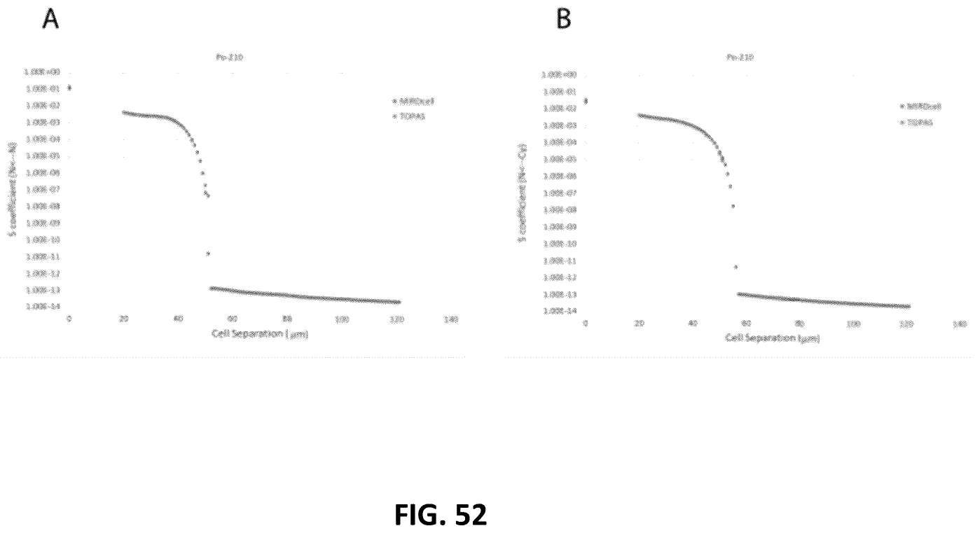

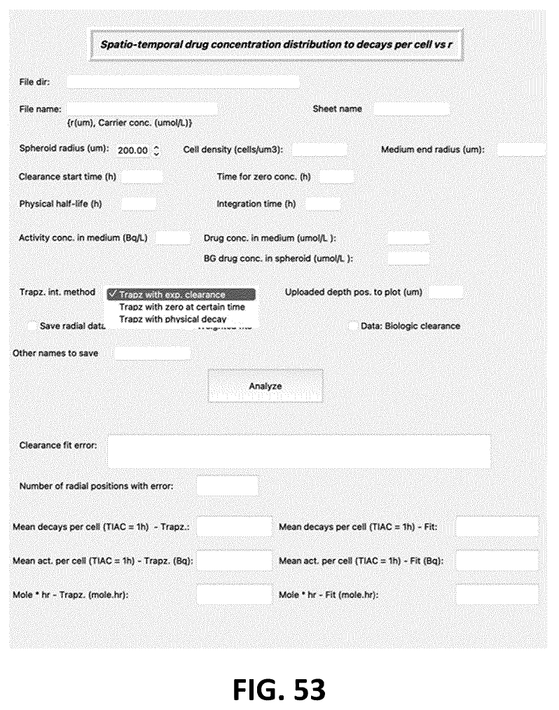

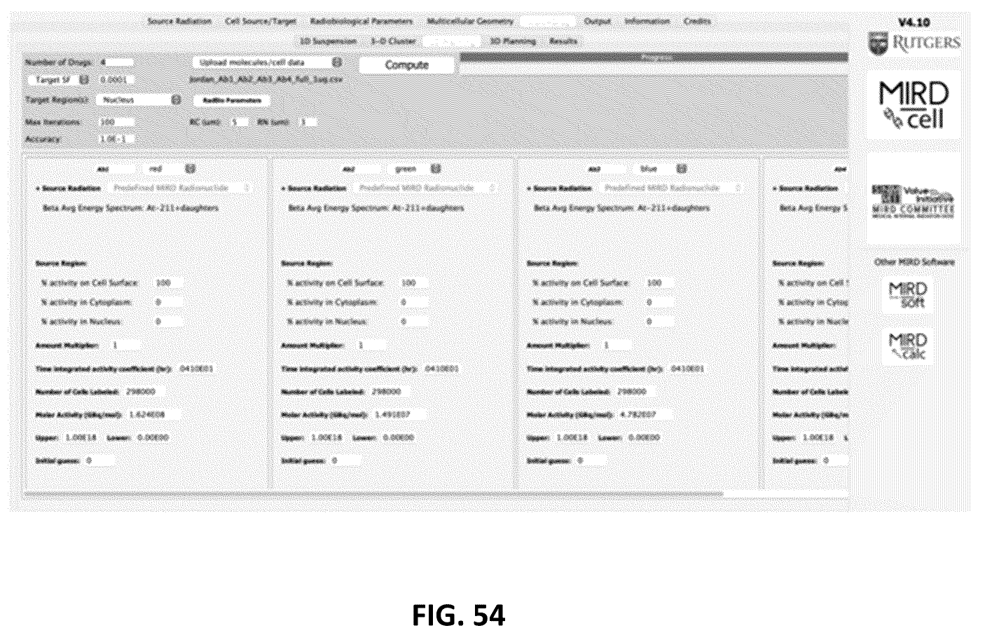

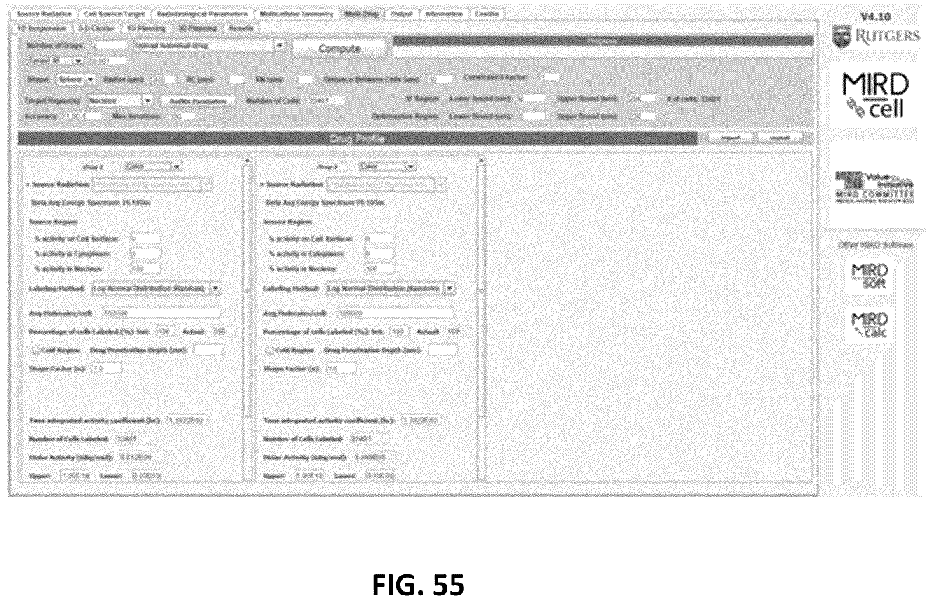

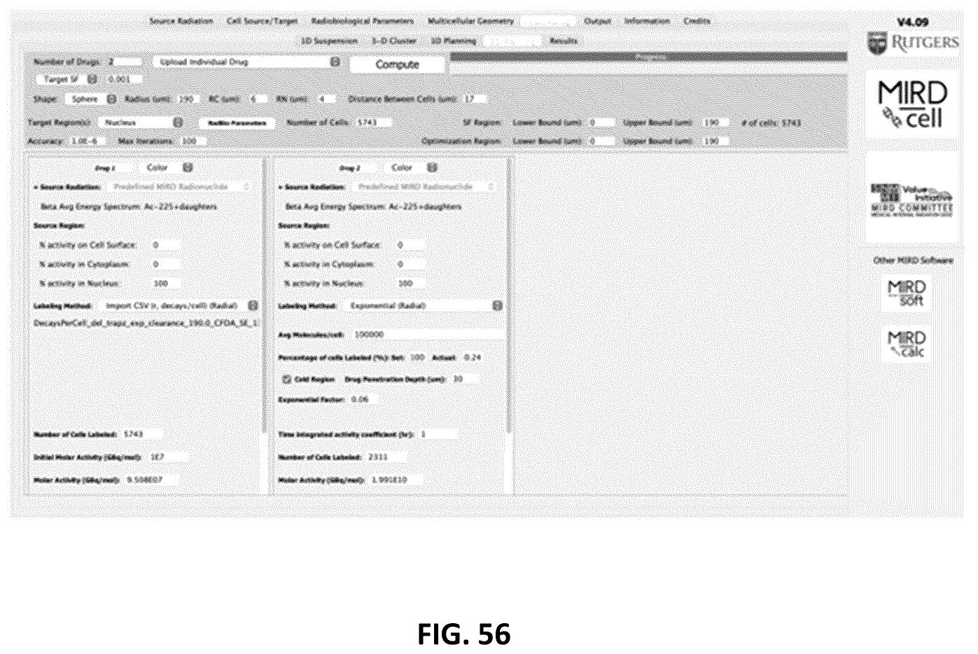

A similar analysis was performed for the alpha emitter ²¹⁰Po using both MIRDcell and TOPAS-nBio. The cell and nucleus dimensions were again kept the same. The results are given in FIG. 52. The production cut used in the TOPAS-nBio simulations was 0.01 μm for both alpha particles and electrons. The tail in the S coefficient curve predicted by MIRDcell for cell separation distances above 52-53 μm is due to the conversion electrons in the ²¹⁰Po spectrum which have a very small yield. The agreement between MIRDcell and TOPAS-nBio is satisfactory for ²¹⁰Po.

Radiopharmaceutical cocktails have been developed to treat cancer. Cocktails of agents are attractive because one radiopharmaceutical is unlikely to have the desired therapeutic effect due to nonuniform uptake by the targeted cells. Therefore, multiple radiopharmaceuticals targeting different receptors of a cell is warranted. However, implementations in vivo have not met with convincing results due to the absence of optimization strategies. In this document, we disclose artificial intelligence (AI) tools housed in a new version of our software platform, MIRDcell V4, that optimize a cocktail of radiopharmaceuticals by minimizing the total disintegrations needed to achieve a given surviving fraction (SF) of tumor cells. Different cells have different receptors to which targeting agents can attach to, causing a heterogeneity in the type and the number of targeting agents attached. Given the distribution of these binding sites for different targeting agents among cells, the molar activity carried by each targeting agent (drug) needs to be optimized to achieve maximum cell kill. The long-term goal of this approach is to determine the optimal drug combination from a sample of cells obtained from a patient and use that information to create a patient-specific cocktail to maximize the therapeutic effect for that patient using the minimum number of disintegrations.

The AI tools are developed as features for MIRDcell V4 which is written in the Java programming language. The optimizer engine for the AI tool is based on the Sequential Least SQuares Programming (SLSQP) algorithm which is written in Fortran. We translated the collection of SLSQP subroutines from Fortran to Java. The Java SLSQP was integrated into our algorithms in MIRDcell that determine the molar activities for each drug that minimize the total disintegrations required for a cocktail of drugs to achieve a specified SF. This approach could be used to analyze a sample of cells obtained from cell culture, an animal, or a patient to predict the best combination of available drugs to be used in the treatment for maximum therapeutic effect with the least total disintegrations, thereby minimizing adverse normal tissue toxicity. Tools are provided for addressing a population of cells that do not cross-irradiate (e.g., circulating or disseminated tumor cells), and for multicellular clusters of cells (e.g., spheroids and micro-metastases). The AI tools were tested using MIRDcell model data as well as experimental data sets.

Combinations of drugs carrying radionuclides have been studied as an attempt to overcome the nonuniformity of absorbed dose distribution within a population of tumor cells. A cocktail of antibodies can target different receptors of a cell population making the complementary labeling of the tumor cells with the different antibodies more effective in achieving homogeneity in labeling. Studies with cocktails of radiolabeled antibodies have been conducted with varying degrees of success. Furthermore, liposomes with tumor penetrating properties have also been studied as carriers of radionuclides with moderate success in controlling tumor growth. Combinations of antibodies and liposomes carrying radionuclides have also provided success in tumor control when compared with either of them being used alone. Although the mixtures of radiopharmaceutical cocktails can be effective in tumor control, few studies have been done to optimize the mixture to minimize the absorbed dose to the normal tissues while achieving the desired therapeutic effect. Therefore, there is a need for methodologies that optimize the constituents of a radiopharmaceutical cocktail without compromising the therapeutic goal.

Even though antibodies with differential reactivities to tumor and normal tissues, differential binding to dissimilar receptors in tumor cells, and radiopharmaceutical cocktails targeting different receptors in tumor cells have been investigated have been studied over the years, their success in vivo has been limited. One of the main contributors to this is the lack of optimization strategies to estimate the “right mix” of each radiopharmaceutical in the cocktail that would achieve the desired therapeutic goal with minimum absorbed dose to the normal tissues. To the best of our knowledge, optimization at the cellular level to treat circulating tumor cells, disseminated tumor cells, and micro-metastases has not been undertaken. Optimizing mixtures of radiopharmaceuticals for treating these need to account for the nonuniform binding characteristics of the vehicles to the cancer cells, and the characteristics of the radiations emitted by the radionuclides that are being delivered, to arrive at optimal molar activities for each radiopharmaceutical in the cocktail.

In this document, we disclose an artificial intelligence (AI) feature that has been added to the MIRDcell software platform to optimize cocktails of radiopharmaceuticals that achieve a predetermined therapeutic goal with the minimum number of disintegrations from all radiopharmaceuticals. The optimization algorithm analyzes the spatial distributions of each radiopharmaceutical and calculates the molar activities of each radiopharmaceutical in the cocktail to achieve the desired surviving fraction of cells while minimizing the total disintegrations in a user defined region. Tools are provided to conduct these analyses for a suspension of cells that represent circulating and disseminated tumor cells, or for a spherical multicellular cluster that represents a spheroid or micro-metastasis. We also disclose examples illustrating the utility of the AI tools using both experimental data and data created to simulate realistic scenarios. These AI tools are a step forward toward developing curative cancer therapies that minimize absorbed dose to normal tissues.

The 1D Planning AI tool can be used to find the optimum combination from a given set of radiopharmaceuticals to achieve a specified therapeutic effect on a population of cells that are sufficiently far apart such that there is no cross absorbed dose from neighboring cells, such as a suspension of cells in culture, circulating tumor cells, or disseminated tumor cells. Only the self-dose (absorbed dose from disintegrations within the cell) is used to calculate the survival probability for each cell. The therapeutic effect, specified as a surviving fraction of the cells (SF), is achieved while minimizing the number of total disintegrations occurring among all the cells.

Each cell has a different number of receptors for each drug that targets a unique receptor. Given the nonuniform distributions of these different receptor sites, the molar activity of each radiopharmaceutical needs to be optimized to realize the desired therapeutic effect while minimizing the total number of disintegrations.

The data to be uploaded to MIRDcell includes the molecules per cell, Mₖⱼ, where the indices k and j represent the k^{th} cell and j^{th} radiopharmaceutical drug, respectively. Such data can be obtained with quantitative flow cytometry when fluorescence can be used. Alternatively, the fluorescence intensity units (FIU/cell) for each drug on each cell can be uploaded along with specifying L, the calibration coefficient for molecules/cell per FIU/cell. Taking the matrix product of this distribution with a column vector parameterizing the product of molar activity, qⱼ, of the j^{th} radiopharmaceutical, and the time integrated activity coefficient, [symbol] , divided by Avogadro's number, N_{A}, results in a vector of total disintegrations Ã(Cₖ) in each cell k.

$$\vert \begin{matrix}\tilde{A} ( {C}_{1} ) \\ ⋮ \\ \tilde{A} ( {C}_{j} ) \\ ⋮ \\ \tilde{A} ( {C}_{n} )\end{matrix} \vert = \vert \begin{matrix}{M}_{11} & … & {M}_{1 j} & … & {M}_{1 m} \\ ⋮ & ⋱ & ⋮ & ⋱ & ⋮ \\ {M}_{k 1} & ⋱ & {M}_{kj} & ⋱ & {M}_{k m} \\ ⋮ & ⋱ & ⋮ & ⋱ & ⋮ \\ {M}_{n 1} & … & {M}_{nj} & … & {M}_{n m}\end{matrix} \vert \times \vert \begin{matrix}\tilde{{a}_{1}} {q}_{1} / {N}_{A} \\ ⋮ \\ \tilde{{a}_{j}} {q}_{j} / {N}_{A} \\ ⋮ \\ \tilde{{a}_{m}} {q}_{m} / {N}_{A}\end{matrix} \vert$$

In the above equation, n is the number of cells in the population, m is the number of radiopharmaceuticals used. This matrix product is calculated for each combination of the set of radiopharmaceuticals used. For example, if there are four radiopharmaceuticals (drug1, drug2, drug3, drug4), the matrix product given in the above equation is calculated for each combination (i.e., drug1, drug2, drug3, drug4, drug1-drug2, drug1-drug3, . . . etc.).

The optimizing algorithm in the 1D Planning AI tool works by minimizing the total number of disintegrations in the entire cell population that are required to achieve a specified target SF, subject to several constraints. The optimization process is performed for each combination of drugs. The optimization algorithm can be summarized as follows.

$$Objective function : {\sum }_{k = 1}^{n} \tilde{A} ( {C}_{k} )$$

$$Constraint 1 : \frac{1}{n} {\sum }_{k = 1}^{n} {P}_{k} = specified SF$$

$$Constraint 2 : {( {\sum }_{k = 1}^{n} \tilde{A} ( {C}_{k} ) )}_{l} \leq [ {( {\sum }_{k = 1}^{n} \tilde{A} ( {C}_{k} ) )}_{1} , {( {\sum }_{k = 1}^{n} \tilde{A} ( {C}_{k} ) )}_{2} , … {( {\sum }_{k = 1}^{n} \tilde{A} ( {C}_{k} ) )}_{12} , … ]$$

where in constraint 2, the subscript 1,2, etc. denotes a subset of the drug combination in question. As an example, consider the three-drug combination of drug1-drug3-drug4. Constraint 2 ensures that total disintegrations resulting from the three-drug combination would remain less than or equal to the minimum of total disintegrations from each of the one- and two-drug combinations of drugs 1, 3 and 4 (i.e., minimum of (Σₖ₌₁ⁿ=1Ã(Cₖ))₁, (Σₖ₌₁ⁿ=1Ã(Cₖ))₃, (Σₖ₌₁ⁿ=1Ã(Cₖ))₄, (Σₖ₌₁ⁿ=1Ã(Cₖ))₁₃, (Σₖ₌₁ⁿ=1Ã(Cₖ))₁₄ and (Σₖ₌₁ⁿ=1Ã(Cₖ))₃₄). The survival probability of each cell Pₖ, is calculated in MIRDcell using a non-interacting Linear Quadratic (LQ) model. The contributions from source regions of the cell (Nucleus (N), Cytoplasm (Cy) and Cell Surface (CS)) are accounted for by tallying the absorbed doses to the nucleus of each cell from each radiation type. It is important to note that the 1D Planning tab uses the self-dose from each radiation type to calculate the survival probability of a cell from that radiation type. The product of the probabilities for each radiation type gives the net probability of surviving the radiation insult from all radiation types. As an example, when the cell nucleus is considered as the source and target and the radiation type is designated by the ICODE, the probability that the k^{th} cell survives is given by the equation below. The ICODEs for several radiation types are given in MIRD: Radionuclide Data and Decay Schemes monograph (28). Following the notation used in MIRD Pamphlet No. 27:

P_{ICODE}(Nₖ)=e^{α}^{_{ICODE}}^{^{SELF}}^{(N}^{ₖ}^{←N}^{ₖ}^{)D} ^{_{ICODE}}^{^{SELF}}^{(N}^{ₖ}^{←N}^{ₖ}^{)-β}^{_{ICODE}}^{^{SELF}}^{(N}^{ₖ}^{←N}^{ₖ}^{) [D}^{_{ICODE}}^{^{SELF}}^{(N}^{ₖ}^{←N}^{ₖ}^{)]}²×e^{α}^{_{ICODE}}^{^{SELF}}^{(N}^{ₖ}^{←Cy}^{ₖ}^{)D} ^{_{ICODE}}^{^{SELF}}^{(N}^{ₖ}^{←Cy}^{ₖ}^{)-β}^{_{ICODE}}^{^{SELF}}^{(N}^{ₖ}^{←Cy}^{ₖ}^{) [D}^{_{ICODE}}^{^{SELF}}^{(N}^{ₖ}^{←Cy}^{ₖ}^{)]}²×e^{α}^{_{ICODE}}^{^{SELF}}^{(N}^{ₖ}^{←CS}^{ₖ}^{)D} ^{_{ICODE}}^{^{SELF}}^{(N}^{ₖ}^{←CS}^{ₖ}^{)-β}^{_{ICODE}}^{^{SELF}}^{(N}^{ₖ}^{←CS}^{ₖ}^{) [D}^{_{ICODE}}^{^{SELF}}^{(N}^{ₖ}^{←CS}^{ₖ}^{)]}² 1

where,

D_{DICODE}^{self}(Nₖ←Nₖ)=f_{N}Ã(Cₖ)S_{ICODE}^{self}(Nₖ←Nₖ)

D_{DICODE}^{self}(Nₖ←Cyₖ)=f_{Cy}Ã(Cₖ)S_{ICODE}^{self}(Nₖ←Cyₖ)

D_{DICODE}^{self}(Nₖ←CSₖ)=f_{CS}Ã(Cₖ)S_{ICODE}^{self}(Nₖ←CSₖ). 2

In the above equation, f_{N}, f_{Cy}, and f_{CS} are the fraction of cell activity in the nucleus, cytoplasm and cell surface, respectively. The quantity Ã(Cₖ) is the time-integrated activity in the k^{th} cell and the S coefficient, S_{ICODE}^{self}(Nₖ←Nₖ), S_{ICODE}^{self}(Nₖ←Cyₖ), S_{ICODE}^{self}(Nₖ←CSₖ), are the self- absorbed doses to the cell nucleus per decay in the nucleus, cytoplasm and cell surface, respectively, from the radiation type designated by ICODE. When all radiation types (i.e., ICODEs) are considered, within the non-interacting LQ model, P(Nk) can be written as:

P(Nₖ)=π_{ICODE=1}^{Number of ICODEs}P_{ICODE}(Nₖ). 3

Once the optimization is completed, the optimized number of total disintegrations from the drug j is divided by its corresponding time-integrated activity coefficient to obtain the optimized total activity Aⱼ^{opt,tot}. The optimized molar activity of drug j, qⱼ^{opt}, is given by:

$${q}_{j}^{o p t} = \frac{{A}_{j}^{opt , tot}}{{mol}_{j}^{t o t}} , \qquad 4$$

where molⱼ^{tot} is the total number of moles of the radionuclide drug j that is summed over all n cells.

The 3D Planning AI tool optimizes the formulation of radiopharmaceuticals for treating spherical multicellular clusters (i.e., spheroids, micro-metastases). The main algorithm used in the 3D optimizer takes a similar approach as the 1D version, however, there is a difference in the manner the total disintegrations are calculated. Unlike the 1D optimizer, the 3D version supports a variety of activity distributions (uniform, normal, log-normal, exponential, linear, and polynomial) in a spherical multicellular cluster geometry. These activity distributions are built in to MIRDcell and can be selected by the user. In addition, experimental radial distributions of disintegrations per cell can be uploaded to the 3D optimizer. One of the main differences in 3D is that, it allows user to specify a region within which the disintegrations are minimized; whereas in 1D, the total disintegrations among all the cells are always minimized. This option in 3D becomes helpful when analyzing spherical clusters bathed in a radioactive medium where the effect of disintegrations in the medium is taken into account. In that situation, the user can instruct the algorithm to minimize the disintegrations in the medium while achieving a user-specified target SF in the multicellular cluster. In this case, only the disintegrations in the user-specified region are considered for constraint 2.

Built-in activity distributions include uniform (random), lognormal (random), normal (random), linear (radial), and exponential (radial). Here, the user specifies the average number of molecules per cell for each drug in the cocktail. A mean activity per cell of 1 mBq is initially assumed and the number of disintegrations in the cell are calculated by multiplying it by the time-integrated activity coefficient. This results in a distribution of disintegrations among cells. The self-absorbed doses to a target region within the cell from the disintegrations in source regions within the cell are calculated as described above using the corresponding S coefficients. In the 3D optimizer, both self- and cross-absorbed doses from each radiation type (ICODE) and source region are used in calculating the survival probability of a given cell with the assumption of a non-interacting LQ model. In the non-interacting LQ model, the survival probabilities that are calculated from the self-doses are multiplied by the probabilities calculated from the cross-doses to evaluate the probability of survival of a given cell. Similar to the 1D optimizer, the 3D version works by minimizing the objective function subject to the two constraints. One key difference in 3D is that only the number of disintegrations in a user-specified region is minimized in the objective function. Another difference in 3D is that at each iteration of the optimizer, the absorbed dose distributions (i.e., for each ICODE from each source region) resulting from a given drug j is scaled by a factor, Xⱼ, until the resulting surviving fraction from all drugs is matched with the specified target SF within a specified accuracy. The initial distribution of disintegrations from a drug j is then multiplied by the final scaling factor Xⱼ, to obtain the optimized distribution of disintegrations for that drug. This is given in the equation below.

$${[ \begin{matrix}\tilde{A} ( {C}_{1} ) \\ ∶ \\ \tilde{A} ( {C}_{n} )\end{matrix} ]}_{j}^{opt} = {X}_{j} \cdot {[ \begin{matrix}\tilde{A} ( {C}_{1} ) \\ ∶ \\ \tilde{A} ( {C}_{n} )\end{matrix} ]}_{j}^{initial}$$

The optimized activity distribution of the drug j is obtained by dividing the optimized disintegrations by the corresponding time-integrated activity coefficient. The total activity from the drug j, APP , in the cluster is obtained by summing the optimized activity over all cells. The 3D optimizer requires the average number of drug molecules per cell Mj for each drug as one of the inputs. This information is used to calculate the optimized molar activity, qⱼ^{opt}, of the radiopharmaceutical (see the equation below).

$${q}_{j}^{o p t} = \frac{{A}_{j , tot}^{o p t} \times {N}_{A}}{( n \times {\overline{M}}_{j} )}$$

where N_{A} is the Avogadro's number.

The 3D AI tool accepts a .csv file containing radial distributions of disintegrations per cell for one or more drugs. The first column contains the radial positions and the latter column(s) contain the disintegrations per cell. The multicellular cluster is divided into concentric shells and the cells within each assigned concentric shell are assigned the same activity per cell determined from the uploaded radial distribution(s). The optimization process minimizes the disintegrations in the user-specified region subject to the two constraints. When an experimental distribution is uploaded, the optimizer requires the user to input the molar activity for each drug that caused the said distributions of disintegrations. At the termination of the 3D optimizer, the initial molar activity of a given drug j (qⱼ^{initial}) is scaled by the scaling factor Xⱼ, to obtain the optimized molar activity of that drug.

qⱼ^{opt}=Xⱼ·qⱼ^{initial}

The 3D Planning tab in MIRDcell requires the uploaded experimental data to be radial distributions of disintegrations per cell in a spherical multicellular cluster (e.g., spheroid or micro-metastasis). However, when the experimental data for the drugs are spatiotemporal distributions of activity or fluorescence intensity, the data needs to be converted to a radial distribution of disintegrations per cell prior to being uploaded to the 3D Planning tab. To accomplish this preprocessing of experimental data, a software tool named MIRDcell-Ã3d has been developed.

MIRDcell-Ã3d accepts an Excel file (.xlsx file) containing a spatiotemporal distribution of drug concentration (in μM) as the raw data file. The spatial distributions at each time point should be along the radial direction of a spherical geometry, the spheroid radius defined by the user. The other input parameters for MIRDcell-Ã3d that are required from the user can be seen in the screenshot shown in FIG. 53. The fractional drug concentration inside the spheroid relative to the outside (medium) of the spheroid is used to obtain the activity concentration at each radial position within the spheroid. This is achieved by multiplying the outer (medium) activity concentration by the corresponding fractional drug concentration. After correcting for physical decay of the radionuclide being used in the drug, the time-integrated activity concentration is calculated at each radial position of the spheroid. MIRDcell-Ã3d supports two main integration methods: a bi-exponential fit and variations of the trapezoidal method. A pure trapezoidal integration or a hybrid method combining the trapezoidal either with the physical decay or with an exponential fit to the clearance data can be selected. At each radial position, the time-integrated activity concentration is converted to a disintegrations per cell and a file containing a radial distribution of disintegrations per cell is saved in .csv format.

Of note, while diffusion kinetics within the tumor are not explicitly taken into account in the MIRDcell optimization process, an experimentally measured 3D spatiotemporal activity distribution can be analyzed with MIRDcell-Ã3d, which integrates time-varying radiopharmaceutical kinetics including diffusion effects. The MIRDcell-Ã3d output can then be uploaded to the 3D Planning tool.

Using MIRDcell modeling, estimates were also made on the best combination of drugs, along with their molar activities, to achieve a user specified SF with minimum total activity. In a first example of modeling using MIRDcell, four antibodies (Ab) were used: Ab1-APC anti-EGFR (AY13), Ab2-AF-488 anti-CD-44 (691534), Ab3-Pacific Blue anti-CD-73 (AD2) and Ab4-PE anti-CD-44 (BJ18). The experimental data consisted of the number of molecules of each antibody on each of 298000 cells following treatment with a cocktail of Ab, each at a concentration of 1 μg/mL. The Multi-Drug <1D Planning tab of MIRDcell V4 was used to create four drugs and the radii of the cell and its nucleus were set to 5 μm and 3 μm, respectively. The radiation type was selected as At-211+daughters and each antibody was distributed on the cell surface. The other parameters used in the optimization are given in Supplemental FIG. 2. The distributions of drug molecules on cells for each drug combination can be seen in the screenshot shown in FIG. 54. The AI tool was run with a target SF of 0.001.

A key feature of the MIRDcell AI tool is that it determines the optimized total number of disintegrations required and the corresponding molar activities for each drug of the different combinations. These are provided in the MIRDcell V4 Output tab and are summarized in the table below.

| | Total | |
|---|---|---|
| | disintegrations | Molar activity |
| Drug combination | required | required (GBq/mole) |
| Ab1 | 4.333 × 10⁸ | 1.6 × 10⁸ |
| Ab2 | 4.489 × 10⁸ | 1.5 × 10⁷ |
| Ab3 | 3.703 × 10⁸ | 4.8 × 10⁷ |
| Ab4 | 2.656 × 10⁹ | 6.7 × 10⁷ |
| Ab1 + Ab2 | 4.333 × 10⁸ | 1.6 × 10⁶, 1.4 × 10⁷ |
| Ab2 + Ab3 | 2.330 × 10⁸ | 4.2 × 10⁶, 1.4 × 10⁷ |
| Ab3 + Ab4 | 2.694 × 10⁸ | 1.7 × 10⁷, 3.4 × 10⁶ |
| Ab1 + Ab3 | 3.694 × 10⁸ | 5.7 × 10⁴, 4.8 × 10⁷ |
| Ab2 + Ab4 | 3.703 × 10⁸ | 1.2 × 10⁷, 4.0 × 10⁻⁹ |
| Ab1 + Ab4 | 3.106 × 10⁸ | 5.9 × 10⁷, 3.9 × 10⁶ |
| Ab1 + Ab2 + Ab3 | 2.314 × 10⁸ | 0, 4.0 × 10⁶, 1.4 × 10⁷ |
| Ab1 + Ab3 + Ab4 | 2.330 × 10⁸ | 6.0 × 10⁷, 2.6 × 10⁶, 1.3 × 10⁶ |
| Ab1 + Ab2 + Ab4 | 2.330 × 10⁸ | 5.2 × 10⁷, 3.1 × 10⁶, 1.0 × 10⁴ |
| Ab2 + Ab3 + Ab4 | 2.330 × 10⁸ | 4.1 × 10⁶, 1.4 × 10⁷, 2.5 × 10⁴ |
| Ab1 + Ab2 + Ab3 + | 2.011 × 10⁸ | 4.0 × 10⁷, 2.1 × 10⁶, 3.8 × 10⁶, |
| Ab4 | | 6.2 × 10⁻¹⁰ |

Ab4 required the most 211At disintegrations when used alone and Ab3 required the least. It is evident that mixing Abl and Ab2 afforded little or no benefit relative to either alone in terms of reducing the total 211At disintegrations required. In contrast, mixing Ab2 and Ab3 had a considerable effect on the total 211At disintegrations needed; it reduced the required disintegrations almost by a factor of two relative to the best of the two drugs. A similar but less prominent effect can be seen between Ab3 and Ab4. In the example provided here, little to no benefit is seen from adding three or more drugs to the mixture. However, it is important to note that this is specific to this example and could change if the drugs had different distributions in the cell population that favored a more complex mixture.

In a second example, the Multi-Drug <3D Planning tab of the MIRDcell V4 software was used to create two radiopharmaceuticals (drugs) radiolabeled with ¹⁹⁵mpt, a prolific Auger electron emitter. The drugs were each distributed lognormally in a multicellular cluster with a spherical geometry (radius=200 μm. An average number of 100,000 drug molecules/cell was set for both drugs and the activity for each was distributed in the nucleus of the cell. The other parameters used in the simulation can be seen in the screenshot shown in FIG. 55. The optimizer was set to run to achieve a target SF=0.001 while minimizing the total number of disintegrations within the cluster.

The total number of disintegrations required to achieve a target SF=0.001 for each combination of two ¹⁹⁵mPt-radiopharmaceuticals that are independently distributed lognormally among cells comprising a spherical multicellular cluster of radius 200 μm are summarized in the table below.

| | Total disintegrations | Molar activity required |
|---|---|---|
| Drug combination | required | (GBq/mole) |
| Drug 1 | 1.7 × 10⁷ | 6.1 × 10⁶ |
| Drug 2 | 1.7 × 10⁷ | 6.0 × 10⁶ |
| Drug 1 + Drug 2 | 1.0 × 10⁷ | 1.8 × 10⁶, 1.9 × 10⁶ |

A third example illustrates the utility of the optimizer when using ²²⁵Ac delivered by two radiopharmaceuticals that provide different radial distributions within a tumor of spherical geometry. The Multi-Drug <3D Planning tab of the MIRDcell V4 software was used to create two drugs transporting ²²⁵Ac within a multicellular sphere of radius 190 μm. The experimental radial distribution of molecules per cell of Stras et al. was used for one of the drugs. The spatiotemporal distribution of disintegrations/cell was calculated for ²²⁵Ac-liposomes based on these data. These data were for liposomes with properties that allowed deeper penetration into the spheroid. However, the outer region of the spheroid had lower disintegrations/cell values due to low liposome concentration closer to the edge of the spheroid. The second drug was distributed exponentially from the outer edge of the spheroid to a depth of 30 μm using a built in feature of MIRDcell. The other parameters used in the MIRDcell AI can be seen in the screenshot shown in FIG. 56. A target SF-0.001 was set and the optimization was run to determine the best combination of the two drugs that would result in the minimum number of disintegrations within the cluster to achieve the required SF.

Drug 2 alone, which had an exponential distribution to a depth of 30 μm from the edge from the spheroid, required the largest number of disintegrations to achieve the required target SF. Drug 1 alone, namely tumor penetrating liposomes required about 42500 disintegrations, which was about 1500 times smaller than that required by Drug 2 alone. However, when both drugs are combined, the optimizer predicts that the same SF could be achieved by roughly half the disintegrations required by Drug 1 alone. This is an important result because, even though Drug1 is quite effective on its own, the required disintegrations could be reduced almost by a factor of two by adding the less effective Drug 2 to the mixture. This could halve the absorbed dose to the surrounding normal tissue.

The reduction in the required number of disintegrations, when the drug mixture was used with the optimized molar activity ratios, was mainly due to the more uniform absorbed dose distribution within the spheroid .. The drop in absorbed dose to the cells from the 225Ac-liposomes (Drug 1) near the edge of the spheroid is compensated by the absorbed dose from Drug 2. The optimizer adjusts the molar activities of each drug to minimize the total disintegrations required to achieve the predefined target SF for the given spatial distributions of the two drugs. With the optimized results, 100% of the cells in the spheroid received at least 4.5 Gy.

The MIRDcell AI tool can also be used to plan treatments with a cocktail of unlabeled primary drugs (e.g., antibodies) followed by administration of a radiolabeled secondary or a cocktail of radiolabeled drug-specific secondaries. For example, consider the former case using the results for the first example. When a cocktail of radiolabeled primary antibodies are used directly at concentrations of 1 μg mL⁻¹ each, the best two-drug combination includes Ab2 and Ab3 with molar activities of 4.2×106 and 1.4×10⁷ GBq mol⁻¹,respectively. Alternatively, one could treat the cells with 1 and 3.2 μug mL⁻¹ of Ab2 and Ab3 (i.e., the ratio of the molar activities for the 1 μg mL⁻¹ case), respectively, followed by a single radiolabeled secondary that produces 4.2×10⁶ GBq mol⁻¹ for Ab2 and Ab3.

The invention has been described via the specific embodiments and examples provided above which, however, do not limit the invention in any way.

The following additional disclosure further describes embodiments of the inventions.

What is claimed is:

1. A computer-implemented method of determining an optimized combination of radiopharmaceutical agents for a therapy, the method comprising:
receiving drug information related to a plurality of candidate radiopharmaceutical agents for treating a plurality of cells, the drug information comprising, for each candidate radiopharmaceutical agent:
a type of radioactivity associated with the candidate radiopharmaceutical agent;
receiving target information related to the plurality of cells, the target information comprising:
the number of receptor sites for each of the plurality of candidate radiopharmaceutical agents; and
dimensions of the plurality of cells;
receiving a target therapeutic effect;
modeling the effectiveness of one or more cocktails on the plurality of cells, each cocktail comprising one or more of the plurality of candidate radiopharmaceutical agents, wherein the modeling includes a Monte Carlo simulation based on the drug information and the target information; and
determining, from cocktails that achieve the target therapeutic effect, an optimal cocktail having a lowest amount of radioactivity within a defined region.

2. The computer-implemented method of claim 1, wherein determining the optimal cocktail having the lowest amount of radioactivity within the defined region comprises minimizing an amount of total disintegrations needed to achieve the target therapeutic effect.

3. The computer-implemented method of claim 1, wherein the target information further comprises a radius and a nuclear radius of at least one of the plurality of cells.

4. The computer-implemented method of claim 1, wherein the target information further comprises geometry of a multicellular cluster comprising the plurality of cells.

5. The computer-implemented method of claim 1, wherein each candidate radiopharmaceutical agent emits one or more types of radiation comprising alpha, beta, gamma, neutron, conversion electron, auger electron, or any combination thereof.

6. The computer-implemented method of claim 1, wherein the one or more types of radiation comprise different radiation emissions with a plurality of yields and energies.

7. The computer-implemented method of claim 1, wherein each type of radioactivity comprises a half-life.

8. The computer-implemented method of claim 1, further comprising receiving the defined region from a user.

9. The computer-implemented method of claim 1, wherein the target therapeutic effect is a surviving fraction of the plurality of cells.

10. The computer-implemented method of claim 1, wherein target radiopharmaceutical agents can be radiolabeled antibodies, nanobodies, peptides, nanoparticles or any other vehicle or compound.

11. The computer-implemented method of claim 1, wherein receiving the target information related to the plurality of cells comprises determining cellular incorporation of a radiopharmaceutical in a cell population on a cell-by-cell basis using a flow cytometer, wherein the flow cytometer measures fluorescence intensity.

12. The computer-implemented method of claim 11, wherein receiving the target information related to the plurality of cells comprises determining cellular incorporation of a radiopharmaceutical in a cell population using a histological sample.

13. The computer-implemented method of claim 1, wherein receiving the target information related to the plurality of cells comprises receiving a spatial distribution of the plurality of candidate radiopharmaceutical agents around the plurality of cells.

14. The method of claim 1, wherein modeling the effectiveness of the one or more cocktails on the plurality of cells comprises modeling the effectiveness using both self- and cross-absorbed doses from each radiation type for each of the plurality of candidate radiopharmaceutical agents.

15. The method of claim 1, wherein modeling the effectiveness of the one or more cocktails on the plurality of cells comprises calculating the probability that a given cell survives using a linear quadratic model, wherein the linear quadratic model comprises separate parameters for each type of radiation.

16. The method of claim 1, wherein modeling the effectiveness of the one or more cocktails on the plurality of cells comprises modeling radioactivity in the nucleus, cytoplasm, and cell surface.

17. A radiation therapy planning system configured to determine a dose of radiation for a patient for radiation therapy treatment planning, the system comprising:
a computing device; and
a computer-readable storage medium in communication with the computing device, wherein the computer-readable storage medium comprises one or more programming instructions that, when executed, cause the computing device to:
receive drug information related to a plurality of candidate radiopharmaceutical agents for treating a plurality of cells, the drug information comprising, for each candidate radiopharmaceutical agent:
a type of radioactivity associated with the candidate radiopharmaceutical agent;
receive target information related to the plurality of cells, the target information comprising:
the number of receptor sites for each of the plurality of candidate radiopharmaceutical agents; and
dimensions of the plurality of cells;
receive a target therapeutic effect;
model the effectiveness of one or more cocktails on the plurality of cells, each cocktail comprising one or more of the plurality of candidate radiopharmaceutical agents, wherein the modeling includes a Monte Carlo simulation based on the drug information and the target information; and
determine from cocktails that achieve the target therapeutic effect, an optimal cocktail having a lowest amount of radioactive decays within a defined region.

18. The radiation therapy planning system of claim 16, wherein the one or more programming instructions that cause the computing device to receive the target information related to the plurality of cells comprise programming instructions that cause the computing device to determine cellular incorporation of a radiopharmaceutical in a cell population on a cell-by-cell basis using a flow cytometer, wherein the flow cytometer measures fluorescence intensity.

19. The method of claim 16, wherein the one or more programming instructions that cause the computing device to model the effectiveness of the one or more cocktails on the plurality of cells comprises programming instructions that cause the computing device to model the effectiveness using both self- and cross-absorbed doses from each radiation type for each of the plurality of candidate radiopharmaceutical agents.

20. The method of claim 16, wherein the one or more programming instructions that cause the computing device to model the effectiveness of the one or more cocktails on the plurality of cells comprises programming instructions that cause the computing device to calculate the probability that a given cell survives using a linear quadratic model, wherein the linear quadratic model comprises separate parameters for each type of radiation.
